(19) United States
(12) Patent Application Publication
(10) Pub. No.: US 2023/0227499 A1
(43) Pub. Date: Jul. 20, 2023

(54) PRODRUGS OF MITOCHONDRIA-TARGETING OLIGOPEPTIDES

(71) Applicant: Stealth Biotherapeutics Inc., Needham, MA (US)

(72) Inventors: Guozhu Zheng, Lexington, MA (US); Pavels Arsenjans, Riga (LV)

(21) Appl. No.: 18/011,358

(22) PCT Filed: Jun. 22, 2021
(86) PCT No.: PCT/US21/38466

§ 371 Date: Dec. 19, 2022

Related U.S. Application Data
(60) Provisional application No. 63/042,148, filed on Jun. 22, 2020.

(51) Int. Cl. C07K 5/11; C07K 5/107

(52) U.S. Cl. CPC C07K 5/1019; C07K 5/1016

(57) ABSTRACT

Disclosed are various prodrugs of Elamipretide.

### RELATED APPLICATION

This application claims the benefit of priority to U.S. Provisional Patent Application No. 63/042,148, filed Jun. 22, 2020.

### BACKGROUND

Through oxidative phosphorylation, mitochondria convert nutrients and oxygen into adenosine triphosphate (ATP), the chemical transporter of energy in most aerobic organisms. The electron transport chain (ETC) of the mitochondria represent the primary source of ATP, as well as a source of reactive oxygen species (ROS). Mitochondrial dysfunction in a cell results in less ATP production and, as a result, insufficient energy to maintain the cell. Such dysfunction also results in excessive ROS production, spiraling cellular injury, and ultimately apoptosis of the cell. Accordingly, mitochondrial dysfunction is a key element believed to be at the root of a variety of serious, debilitating diseases.

Natural antioxidants, such as coenzyme Q and vitamin E, have been shown to provide some protection of the cell from damage induced by the elevated ROS levels associated with mitochondrial dysfunction. However, antioxidants or oxygen scavengers have also been shown to reduce ROS to unhealthy levels and may not reach the ETC in sufficient concentrations to correct the mitochondrial imbalance. Therefore, there is a need for novel compounds that can selectively target the ETC, restore efficient oxidative phosphorylation, and thereby address mitochondrial disease and dysfunction.

### SUMMARY

Disclosed are prodrugs of mitochondria-targeting oligopeptide compounds. In some embodiments, the oligopeptide compound is Elamipretide (MTP-131; D-Arg-Dmt-Lys-Phe-NH₂).

In some embodiments, the invention provides compounds of Formula (I)

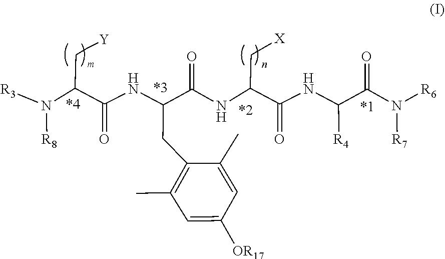

wherein:

X is —N(R₁₅)—R₁,

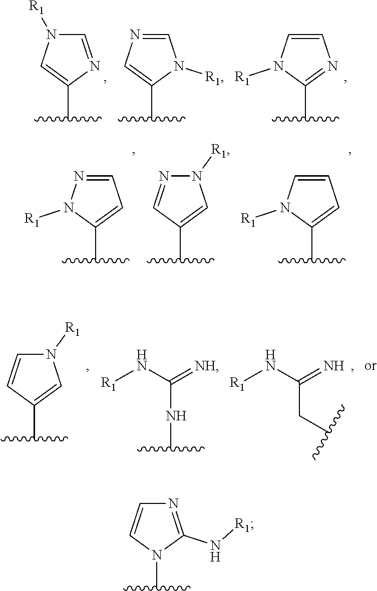

Y is —N(R₁₅)—R₂,

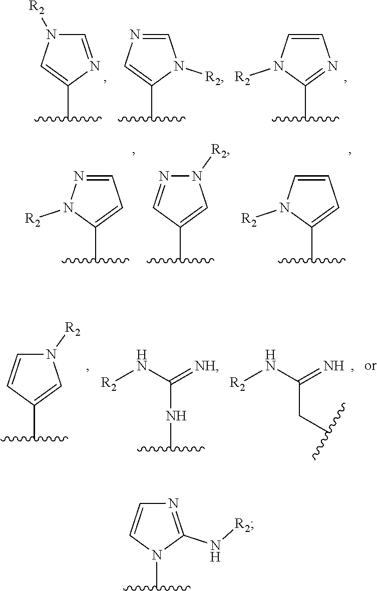

R₁, R₂, R₃, and R₁₇ are independently H, alkyl, alkenyl, alkynyl, aryl, arylalkyl, arylheteroalkyl, cycloalkyl, heteroalkyl, heteroaryl, T, R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₀O)(R₁₂O)P(O)—, or R₁₁R₁₂N(R₉O)P(O)—;

R₄ is alkyl, cycloalkyl, aryl, arylalkyl, heteroaryl, arylheteroalkyl, T, a side-chain of a naturally or non-naturally occurring chiral amino acid,

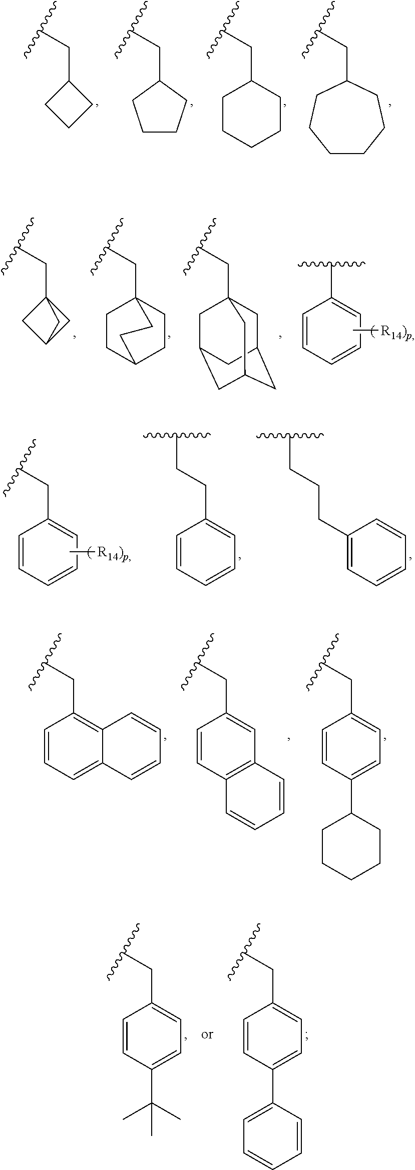

R₆ and R₇ are independently H, alkyl, or acyl; or R₆ and R₇ together with the nitrogen atom to which they are attached form a 4-6-membered heterocyclic ring;

R₈ is H, alkyl, heteroalkyl, or acyl;

R₉, R₁₁, and R₁₂ are independently H, alkyl, alkenyl, alkynyl, heteroalkyl, cycloalkyl, aryl, arylalkyl, heteroaryl, arylheteroalkyl, heteroarylheteroalkyl, or T;

R₁₁ and R₁₂ can be taken together to form a heterocyclic ring;

R₁₀ is alkyl, alkenyl, alkynyl, heteroalkyl, cycloalkyl, aryl, arylalkyl, heteroaryl, arylheteroalkyl, heteroarylheteroalkyl, or T;

R₁₃ is H, methyl, ethyl, isopropyl, or tert-butyl;

R₁₄ is independently D, F, Cl, Br, I, —CH₃, —OCH₃, CH₂CH₃, —OCH₂CH₃, —CCl₃, —CF₃, —OH, or —NO₂;

T is —(CH₂)_{w}—(O)ₓ—[(CH₂CH₂)—O]_{q}—R₁₃;

n and m are independently 1, 2, 3, 4, 5, or 6;

p is 0, 1, 2, 3, 4, or 5;

q is an integer from 1-30 inclusive;

x is 0 or 1; w is 0, 1 or 2; provided that: if x is 0 then w is 0; if w is 0, then x is 0;

the absolute stereochemistry at each of stereocenters 0.1, 0.2, *3 and *4 is independently

R (D for an amino acid) or S (L for an amino acid); and

at least one of R₁, R₂, R₃ and R₁₇ is R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₁O)(R₁₂O)P(O)—, or R₁₁R₁₂N(R₉O)P(O)—.

In some embodiments, the invention provides compounds of Formula (II)

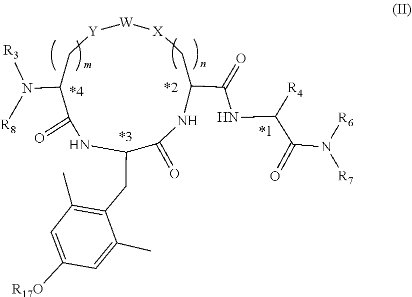

wherein:

X is —N(R₁₅)—,

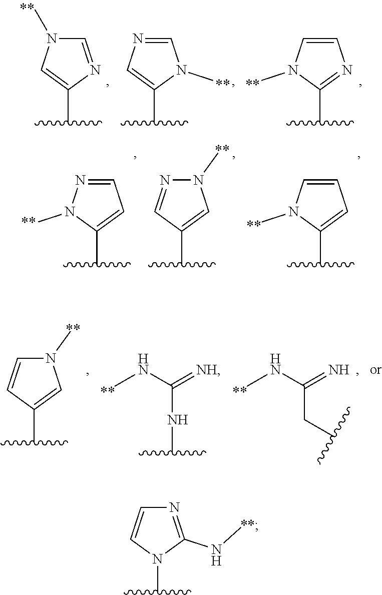

Y is —N(R₁₅)—,

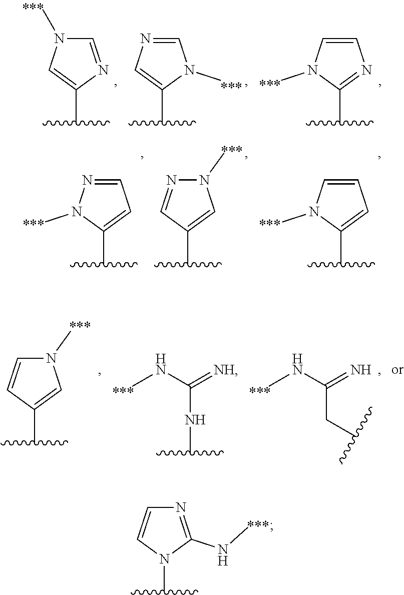

W is —C(O)—, —C(S)—, —C(R₁₆)₂—, —S(O)—, —S(O₂)—, or —P(O)[Q(R₁₀)]—;

Q is O or a bond;

R₃ and R₁₇ are independently H, alkyl, alkenyl, alkynyl, aryl, arylalkyl, arylheteroalkyl, cycloalkyl, heteroalkyl, heteroaryl, T, R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₁O)(R₁₂O)P(O)—, or R₁₁R₁₂N(R₉O)P(O)—;

R₄ is alkyl, cycloalkyl, aryl, arylalkyl, heteroaryl, arylheteroalkyl, T, a side-chain of a naturally or non-naturally occurring chiral amino acid,

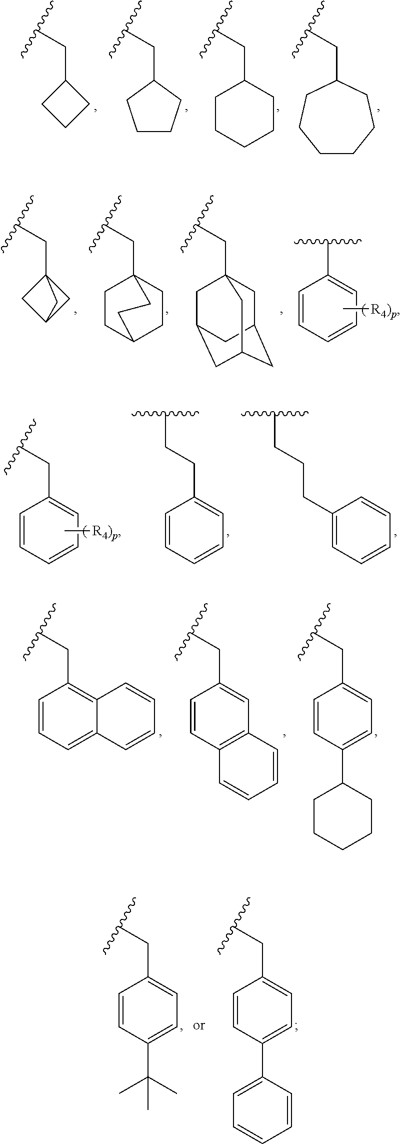

R₆ and R₇ are independently H, alkyl, or acyl; or R₆ and R₇ together with the nitrogen atom to which they are attached form a 4-6-membered heterocyclic ring;

R₈ is H, alkyl, heteroalkyl, or acyl;

R₉, R₁₁, and R₁₂ are independently H, alkyl, alkenyl, alkynyl, heteroalkyl, cycloalkyl, aryl, arylalkyl, heteroaryl, arylheteroalkyl, heteroarylheteroalkyl, or T;

R₁₁ and R₁₂ can be taken together to form a heterocyclic ring;

R₁₀ is alkyl, alkenyl, alkynyl, heteroalkyl, cycloalkyl, aryl, arylalkyl, heteroaryl, arylheteroalkyl, heteroarylheteroalkyl or T;

R₁₃ is H, methyl, ethyl, isopropyl or tert-butyl;

R₁₄ is independently D, F, Cl, Br, I, —CH₃, —OCH₃, CH₂CH₃, —OCH₂CH₃, —CCl₃, —CF₃, —OH, or —NO₂;

R₁₅ is H, alkyl, alkenyl, alkynyl, cycloalkyl, heteroalkyl, or acyl;

R₁₆ is alkyl, alkenyl, alkynyl, heteroalkyl, cycloalkyl, aryl, or arylalkyl;

T is —(CH₂)_{w}—(O)ₓ—[(CH₂CH₂)—O]_{q}—R₁₃;

the absolute stereochemistry at each of stereocenters 0.1, 0.2, *3 and *4 is independently

R (D for an amino acid) or S (L for an amino acid);

n and m are independently 1, 2, 3, 4, 5, or 6;

p is 0, 1, 2, 3, 4, or 5;

q is an integer from 1-30 inclusive;

x is 0 or 1; and

w is 0, 1 or 2; provided that: if x is 0, then w is 0; and if w is 0, then y is 0;

“**” denotes the point of attachment of X to W; and

“***” denotes the point of attachment of W to Y.

### BRIEF DESCRIPTION OF THE DRAWINGS

FIGS. 1A-1C depict that D-Arg-Dmt-Lys-Phe-NH₂ (SS-31; MTP-131) inhibits mitochondrial swelling and cytochrome c release. FIG. 1A shows that the pretreatment of isolated mitochondria with SS-31 (10 μM) prevents onset of MPT induced by ca2+. Gray line, buffer; red line, SS-31. FIG. 1B shows that the pretreatment of mitochondria with SS-31 (50 μM) inhibited mitochondrial swelling induced by 200 mM Ca2+. Swelling was measured by light scattering measured at 570 nm. FIG. 1 C depicts the comparison of SS-02 and SS-31 with cyclosporine (CsA) in inhibiting mitochondrial swelling and cytochrome c release induced by Ca2+. The amount of cytochrome c released was expressed as percent of total cytochrome c in mitochondria. Data are presented as mean±s.e., n=3.

FIG. 2 depicts that 2′,6′-Dmt-D-Arg-PheLys-NH₂ (SS-02) and D-Arg-Dmt-Lys-Phe-NH₂ (SS-31; MTP-131) protects myocardial contractile force during ischemia-reperfusion in the isolated perfused guinea pig heart. Hearts were perfused with buffer or buffer containing SS-02 (100 nM) or SS-31 (1 nM) for 30 min and then subjected to 30-min global ischemia. Reperfusion was carried out using the same perfusion solution. Significant differences were found among the three treatment groups (2-way ANOVA, P<0.001).

### DETAILED DESCRIPTION

The present invention features prodrugs of mitochondria-targeting oligopeptide compounds. In some embodiments, the oligopeptide compound is

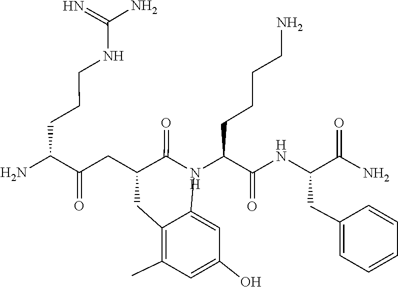 [SMILES: Cc1cc(O)cc(C)c1C[C@H](CC(=O)[C@H](N)CCCNC(=N)N)C(=O)N[C@@H](CCCCN)C(=O)N[C@@H](Cc1ccccc1)C(N)=O]

(MTP-131; D-Arg-DMT-Lys-Phe-NH₂) or a salt thereof. D-Arg-DMT-Lys-Phe-NH₂ has been shown to affect the mitochondrial disease process by helping to protect organs from oxidative damage caused by excess ROS production, and to restore normal ATP production.

In some embodiments, the invention provides compounds of Formula (I)

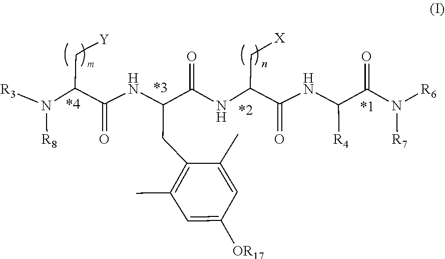

wherein:

X is —N(R₁₅)—R₁,

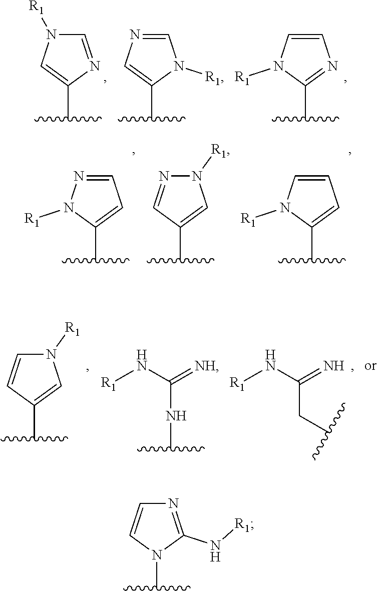

Y is —N(R₁₅)—R₂,

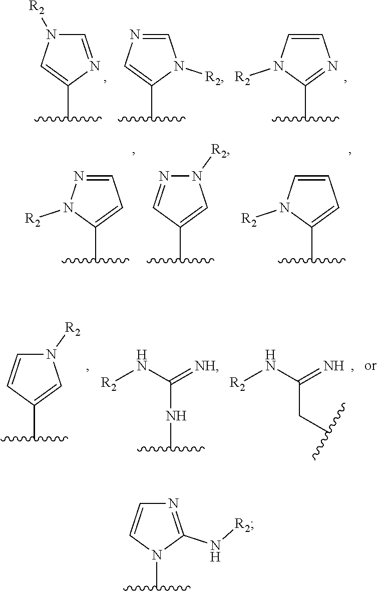

R₁, R₂, R₃, and R₁₇ are independently H, alkyl, alkenyl, alkynyl, aryl, arylalkyl, arylheteroalkyl, cycloalkyl, heteroalkyl, heteroaryl, T, R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₀O)(R₁₂O)P(O)—, or R₁₁R₁₂N(R₉O)P(O)—;

R₄ is alkyl, cycloalkyl, aryl, arylalkyl, heteroaryl, arylheteroalkyl, T, a side-chain of a naturally or non-naturally occurring chiral amino acid,

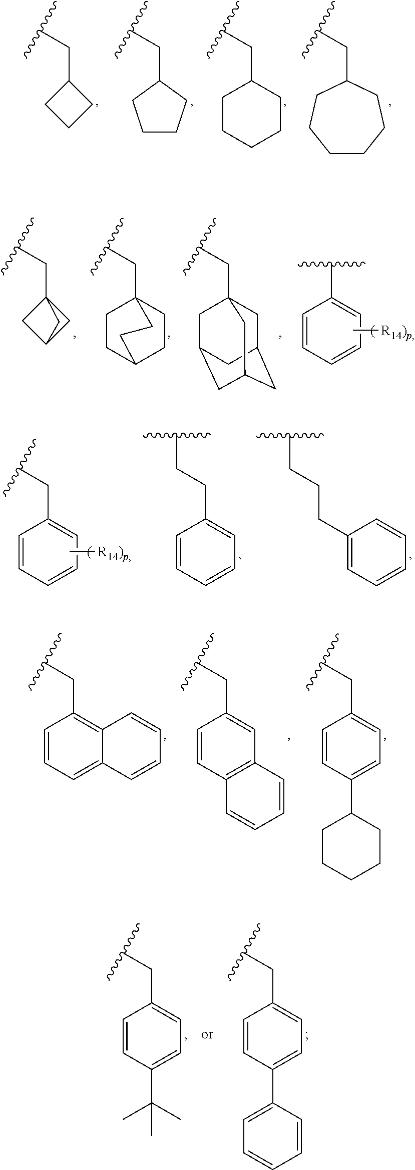

R₆ and R₇ are independently H, alkyl, or acyl; or R₆ and R₇ together with the nitrogen atom to which they are attached form a 4-6-membered heterocyclic ring;

R₈ is H, alkyl, heteroalkyl, or acyl;

R₉, R₁₁, and R₁₂ are independently H, alkyl, alkenyl, alkynyl, heteroalkyl, cycloalkyl, aryl, arylalkyl, heteroaryl, arylheteroalkyl, heteroarylheteroalkyl, or T;

R₁₁ and R₁₂ can be taken together to form a heterocyclic ring;

R₁₀ is alkyl, alkenyl, alkynyl, heteroalkyl, cycloalkyl, aryl, arylalkyl, heteroaryl, arylheteroalkyl, heteroarylheteroalkyl, or T;

R₁₃ is H, methyl, ethyl, isopropyl, or tert-butyl;

R₁₀ is independently D, F, Cl, Br, I, —CH₃, —OCH₃, CH₂CH₃, —OCH₂CH₃, —CCl₃, —CF₃, —C≡N, —OH, or —NO₂;

T is —(CH₂)_{w}—(O)ₓ—[(CH₂CH₂)—O]_{q}—R₁₃;

the absolute stereochemistry at each of stereocenters 0.1, 0.2, *3 and *4 is independently

R (D for an amino acid) or S (L for an amino acid);

n and m are independently 1, 2, 3, 4, 5, or 6;

p is 0, 1, 2, 3, 4, or 5;

q is an integer from 1-30 inclusive;

x is 0 or 1; and

w is 0, 1 or 2; provided that: if x is 0 then w is 0; and if w is 0, then x is 0; and

at least one of R₁, R₂, R₃ and R₁₇ is R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₁O)(R₁₂O)P(O)—, or R₁₁R₁₂N(R₉O)P(O)—.

In some embodiments, X is —N(R₁₅)R₁. In some embodiments, X is

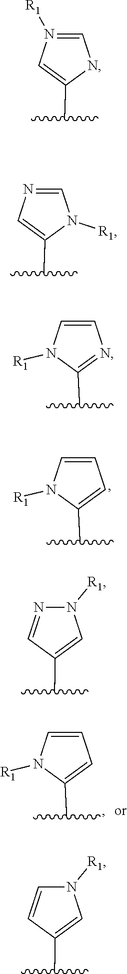

In some embodiments, X is

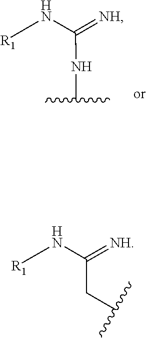

In some embodiments, X is

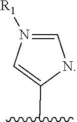

In some embodiments, X is

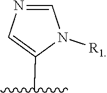

In some embodiments, X is

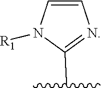

In some embodiments, X is

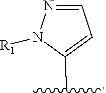

In some embodiments, X is

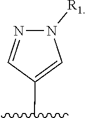

In some embodiments, X is

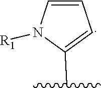

In some embodiments, X is

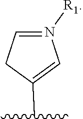

In some embodiments, X is

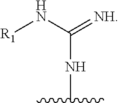

In some embodiments, X is

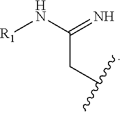

In some embodiments, X is

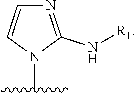

In some embodiments, Y is —N(R₁₅)—R₂. In some embodiments, Y is

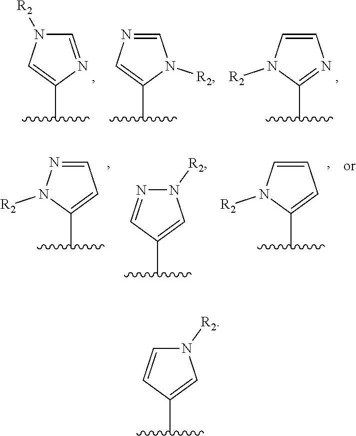

In some embodiments, Y is

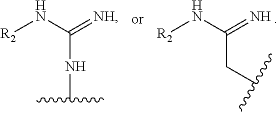

In some embodiments, Y is

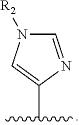

In some embodiments, Y is

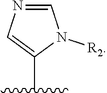

In some embodiments, Y is

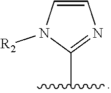

In some embodiments, Y is

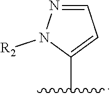

In some embodiments, Y is

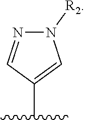

In some embodiments, Y is

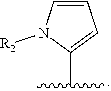

In some embodiments, Y is

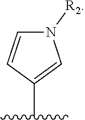

In some embodiments, Y is

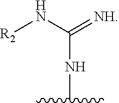

In some embodiments, Y is

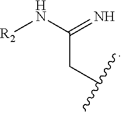

In some embodiments, Y is

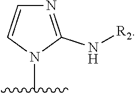

In some embodiments, R₁ is H. In some embodiments, R₁ is alkyl, alkenyl, alkynyl, aryl, arylalkyl, arylheteroalkyl, cycloalkyl, heteroalkyl, or heteroaryl. In some embodiments, R₁ is C₁-C₈ alkyl. In some embodiments, R₁ is a C₁-C₈ alkenyl, alkynyl, aryl, arylalkyl, arylheteroalkyl, cycloalkyl, heteroalkyl, or heteroaryl group. In some embodiments, R₁ is heteroalkyl. In some embodiments, R₁ is T. In some embodiments, R₁ is —[(CH₂CH₂)—O]_{q}—R₁₃. In some embodiments, R₁ is R₉C(O)—, R₁₀OC(O)—, or (R₁₁O)(R₁₂O)P(O)—. In some embodiments, R₁ is R₉C(O)—. In some embodiments, R₁ is CH₃C(O)—. In some embodiments, R₁ is T-C(O)—. In some embodiments, R₁ is R₁₃—[O—(CH₂CH₂)]_{q}—C(O)—. In some embodiments, R₁ is CH₃—O—CH₂CH₂—C(O)—. In some embodiments, R₁ is CH₃—O—CH₂CH₂—O—CH₂—C(O)—. In some embodiments R₁ is R₁₀OC(O)—. In some embodiments, R₁ is CH₃CH₂OC(O)—. In some embodiments, R₁ is R₁₃—[O—(CH₂CH₂)]_{q}—O—C(O)—. In some embodiments, R₁ is CH₃—[O—(CH₂CH₂)]_{q}—O—C(O)—. In some embodiments, R₁ is CH₃—[O—(CH₂CH₂)]₇—O—C(O)—. In some embodiments, R₁ is (R₁₁O)(R₁₂O)P(O)—. In some embodiments, R₁ is (R₁₃—[O—(CH₂CH₂)]_{q}—O—)(R₁₃—[O—(CH₂CH₂)]_{q}—O—)P(O)—. In some embodiments, R₁ is (CH₃—O—(CH₂CH₂)ₖ—O—)(CH₃—[O—(CH₂CH₂)]_{q}—O—)P(O)—. In some embodiments, R₁ is (CH₃[O—(CH₂CH₂)]_{q}—O—)(CH₃—[O—(CH₂CH₂)]₇—O)P(O)—. In some embodiments, R₁ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O). In some embodiments, R₁ is R₁₁R₁₂NC(O)—. In some embodiments, R₁ is R₁₀S(O)—. In some embodiments, R₁ is R₁₀S(O)₂—. In some embodiments, R₁ is R₁₀OS(O)—. In some embodiments, R₁ is R₁₀OS(O)₂—. In some embodiments, R₁ is R₁₁R₁₂N(R₉O)P(O). In some embodiments, R₁ is not Cbz, Boc, Bpoc, Nps, Ddz, Fmoc, ivDde, Msc, Nsc, Bsmoc, Sps, or Esc.

In some embodiments, R₂ is H. In some embodiments, R₂ is alkyl, alkenyl, alkynyl, aryl, arylalkyl, arylheteroalkyl, cycloalkyl, heteroalkyl, or heteroaryl. In some embodiments, R₂ is C₁-C₈ alkyl. In some embodiments, R₂ is a C₁-C₈ alkenyl, alkynyl, aryl, arylalkyl, arylheteroalkyl, cycloalkyl, heteroalkyl, or heteroaryl group. In some embodiments, R₂ is heteroalkyl. In some embodiments, R₂ is T. In some embodiments, R₂ is —[(CH₂CH₂)—O]_{q}—R₁₃. In some embodiments, R₂ is R₉C(O)—, R₁₀OC(O)—, or (R₁₁O)(R₁₂O)P(O)—. In some embodiments, R₂ is R₉C(O)—. In some embodiments, R₂ is CH₃C(O)—. In some embodiments, R₂ is T-C(O)—. In some embodiments, R₂ is R₁₃—[O—(CH₂CH₂)]_{q}—C(O)—. In some embodiments, R₂ is CH₃—O—CH₂CH₂—C(O)—. In some embodiments, R₂ is CH₃—O—CH₂CH₂—O—CH₂—C(O)—. In some embodiments R₂ is R₁₀OC(O)—. In some embodiments, R₂ is CH₃CH₂OC(O)—. In some embodiments, R₂ is R₁₃—[O—(CH₂CH₂)]_{q}—O—C(O)—. In some embodiments, R₂ is CH₃—[O—(CH₂CH₂)]_{q}—O—C(O)—. In some embodiments, R₂ is CH₃—[O—(CH₂CH₂)]₇—O—C(O)—. In some embodiments, R₂ is (R₁₁O)(R₁₂O)P(O)—. In some embodiments, R₂ is (R₁₃—[O—(CH₂CH₂)]_{q}—O—)(R₁₃—[O—(CH₂CH₂)]_{q}—O—)P(O)—. In some embodiments, R₂ is (CH₃—[O—(CH₂CH₂)]_{q}—O—)(CH₃—[O—(CH₂CH₂)]_{q}—O—)P(O)—. In some embodiments, R₂ is (CH₃—[O—(CH₂CH₂)]₇—O)(CH₃—[O—(CH₂CH₂)]7-O)P(O)—. In some embodiments, R₂ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O). In some embodiments, R₂ is R₁₁R₁₂NC(O)—. In some embodiments, R₂ is R₁₀S(O)—. In some embodiments, R₂ is R₁₀S(O)₂—. In some embodiments, R₂ is R₁₀OS(O)—. In some embodiments, R₂ is R₁₀OS(O)₂—. In some embodiments, R₂ is R₁₁R₁₂N(R₉O)P(O). In some embodiments, R₂ is not Cbz, Boc, Bpoc, Nps, Ddz, Fmoc, ivDde, Msc, Nsc, Bsmoc, Sps, or Esc.

In some embodiments, R₃ is H. In some embodiments, R₃ is alkyl, alkenyl, alkynyl, aryl, arylalkyl, arylheteroalkyl, cycloalkyl, heteroalkyl, or heteroaryl. In some embodiments, R₃ is C₁-C₈ alkyl. In some embodiments, R₃ is a C₁-C₈ alkenyl, alkynyl, aryl, arylalkyl, arylheteroalkyl, cycloalkyl, heteroalkyl, or heteroaryl group. In some embodiments, R₃ is heteroalkyl. In some embodiments, R₃ is T. In some embodiments, R₃ is —[(CH₂CH₂)—O]_{q}—R₁₃. In some embodiments, R₃ is R₉C(O)—, R₁₀OC(O)—, or (R₁₁O)(R₁₂O)P(O)—. In some embodiments, R₃ is R₉C(O)—. In some embodiments, R₃ is CH₃C(O)—. In some embodiments, R₃ is T-C(O)—. In some embodiments, R₃ is R₁₃—[O—(CH₂CH₂)]_{q}—C(O)—. In some embodiments, R₃ is CH₃—O—CH₂CH₂—C(O)—. In some embodiments, R₃ is CH₃—O—CH₂CH₂—O—CH₂—C(O)—. In some embodiments R₃ is R₁₀OC(O)—. In some embodiments, R₃ is CH₃CH₂OC(O)—. In some embodiments, R₃ is R₁₃[O—(CH₂CH₂)]_{q}—O—C(O)—. In some embodiments, R₃ is CH₃—[O—(CH₂CH₂)]_{q}—O—C(O)—. In some embodiments, R₃ is CH₃—[O—(CH₂CH₂)]₇—O—C(O)—. In some embodiments, R₃ is (R₁₁O)(R₁₂O)P(O)—. In some embodiments, R₃ is (R₁₃—[O—(CH₂CH₂)]_{q}—O—)(R₁₃—[O—(CH₂CH₂)]_{q}—O—)P(O)—. In some embodiments, R₃ is (CH₃—[O—(CH₂CH₂)]_{q}—O—)(CH₃—[O—(CH₂CH₂)]_{q}—O—)P(O)—. In some embodiments, R₃ is (CH₃—[O—(CH₂CH₂)]7-O)(CH₃—[O—(CH₂CH₂)]7-O)P(O)—. In some embodiments, R₃ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O). In some embodiments, R₃ is R₁₁R₁₂NC(O)—. In some embodiments, R₃ is R₁₀S(O)—. In some embodiments, R₃ is R₁₀S(O)₂—. In some embodiments, R₃ is R₁₀OS(O)—. In some embodiments, R₃ is R₁₀OS(O)₂—. In some embodiments, R₃ is R₁₁R₁₂N(R₉O)P(O). In some embodiments, R₃ is not Cbz, Boc, Bpoc, Nps, Ddz, Fmoc, ivDde, Msc, Nsc, Bsmoc, Sps, or Esc.

In some embodiments, R₄ is alkyl, cycloalkyl, aryl, arylalkyl, heteroaryl, or arylheteroalkyl. In some embodiments, R₄ is T. In some embodiments, R₅ is a side-chain of a naturally or non-naturally occurring chiral amino acid. In some embodiments, R₄ is

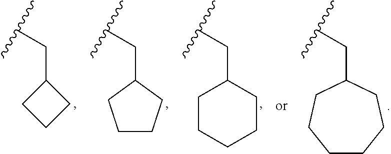

In some embodiments, R₄ is

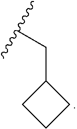 [SMILES: *CC1CCC1]

In some embodiments, R₄ is

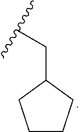 [SMILES: *CC1CCCC1]

In some embodiments, R₄ is

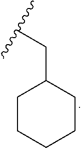 [SMILES: *CC1CCCCC1]

In some embodiments, R₄ is

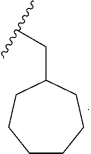 [SMILES: *CC1CCCCCC1]

In some embodiments, R₄ is

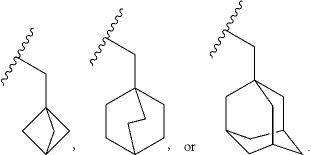

In some embodiments, R₄ is

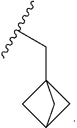 [SMILES: *CC12CC(C1)C2]

In some embodiments, R₄ is

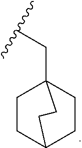 [SMILES: *CC12CCC(CC1)CC2]

In some embodiments, R₄ is

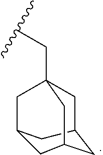 [SMILES: *CC12CC3CC(CC(C3)C1)C2]

In some embodiments, R₄ is

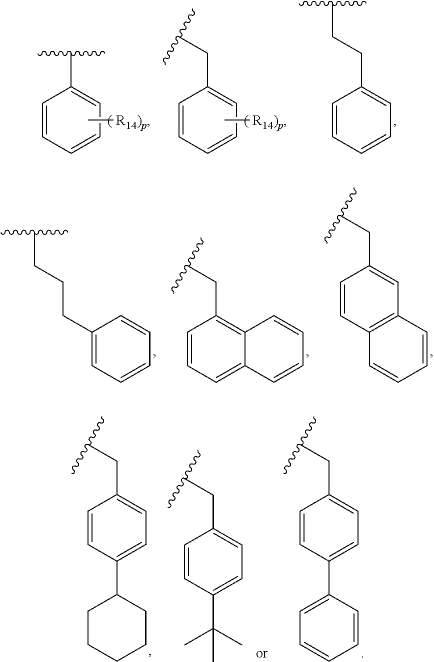

In some embodiments, R₄ is

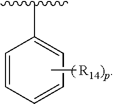

In some embodiments, R₄ is

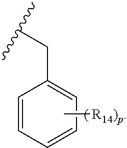

In some embodiments, R₄ is

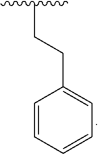 [SMILES: *CCc1ccccc1 ; "."]

In some embodiments, R₄ is

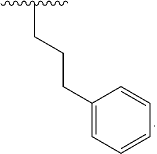 [SMILES: *CCCc1ccccc1 ; "."]

In some embodiments, R₄ is

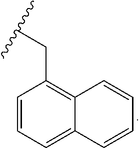 [SMILES: *Cc1cccc2ccccc12 ; "."]

In some embodiments, R₄ is

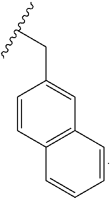 [SMILES: *Cc1ccc2ccccc2c1]

In some embodiments, R₄ is

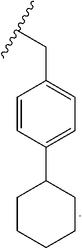 [SMILES: *Cc1ccc(C2CCCCC2)cc1]

In some embodiments, R₄ is

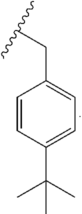 [SMILES: *Cc1ccc(C(C)(C)C)cc1]

In some embodiments, R₄ is

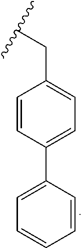 [SMILES: *Cc1ccc(-c2ccccc2)cc1]

In some embodiments, R₄ is —R₁₃, —OR₁₃ or

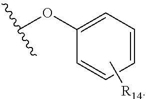

In some embodiments, R₄ is —R₁₃. In some embodiments, R₄ is —OR₁₃. In some embodiments, R₄ is

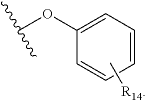

In some embodiments, R₄ is

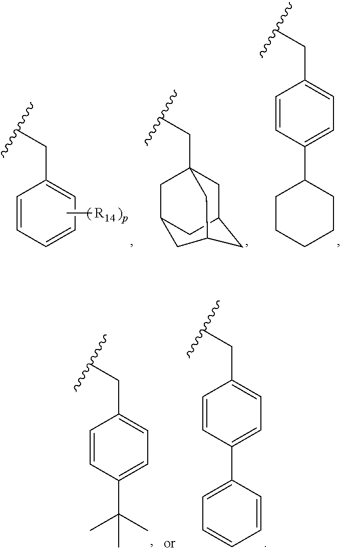

In some embodiments, R₄ is

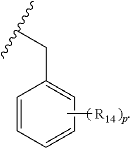

In some embodiments, R₄ is

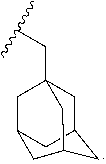 [SMILES: *CC12CC3CC(CC(C3)C1)C2]

In some embodiments, R₄ is

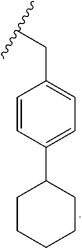 [SMILES: *Cc1ccc(C2CCCCC2)cc1]

In some embodiments, R₄ is

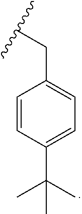 [SMILES: *Cc1ccc(C(C)(C)C)cc1]

In some embodiments, R₄ is

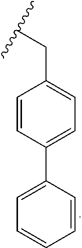 [SMILES: *Cc1ccc(-c2ccccc2)cc1]

In some embodiments, R₈ is H. In some embodiments, R₈ is alkyl, heteroalkyl, or acyl. In some embodiments, R₈ is C₁-C₈ alkyl. In some embodiments, R₈ is C₁-C₁₅ heteroalkyl. In some embodiments, R₈ is H, methyl or ethyl.

In some embodiments, R₉ is H. some embodiments, R₉ is alkyl, alkenyl, alkynyl, heteroalkyl, cycloalkyl, aryl, arylalkyl, heteroaryl, arylheteroalkyl, or heteroarylheteroalkyl. In some embodiments, R₉ is C₁-C₈ alkyl. In some embodiments, R₉ is C₁-C₁₅ heteroalkyl. In some embodiments, R₉ is T. In some embodiments, R₉ is —[(CH₂CH₂)—O]_{q}—R₁₃ and q is 1-20.

In some embodiments, R₁₀ is alkyl, alkenyl, alkynyl, heteroalkyl, cycloalkyl, aryl, arylalkyl, heteroaryl, arylheteroalkyl, or heteroarylheteroalkyl. In some embodiments, R₁₀ is C₁-C₈ alkyl. In some embodiments, R₁₀ is C₁-C₁₅ heteroalkyl. In some embodiments, R₁₀ is T. In some embodiments, R₁₀ is —[(CH₂CH₂)—O]_{q}—R₁₃ and q is 1-20.

In some embodiments, R₁₁ is H. In some embodiments, R₁₁ is alkyl, alkenyl, alkynyl, heteroalkyl, cycloalkyl, aryl, arylalkyl, heteroaryl, arylheteroalkyl, or heteroarylheteroalkyl. In some embodiments, R₁₁ is C₁-C₈ alkyl. In some embodiments, R₁₁ is C₁-C₁₅ heteroalkyl. In some embodiments, R₁₁ is T. In some embodiments, R₁₁ is —[(CH₂CH₂)—O]_{q}—R₁₃ and q is 1-20.

In some embodiments, R₁₂ is H. In some embodiments, R₁₂ is alkyl, alkenyl, alkynyl, heteroalkyl, cycloalkyl, aryl, arylalkyl, heteroaryl, arylheteroalkyl, or heteroarylheteroalkyl. In some embodiments, R₁₂ is C₁-C₈ alkyl. In some embodiments, R₁₂ is C₁-C₁₅ heteroalkyl. In some embodiments, R₁₂ is T. In some embodiments, R₁₂ is —[(CH₂CH₂)—O]_{q}—R₁₃ and q is 1-20.

In some embodiments, R₁₁ and R₁₂ are taken together to form a heterocyclic ring. In some embodiments, the heterocyclic ring is a 3-membered to 7-membered ring. The heterocyclic ring can be substituted or unsubstituted.

In some embodiments, R₁₃ is H. In some embodiments, R₁₃ is methyl, ethyl, isopropyl or tert-butyl.

In some embodiments, R₁₄ is deuterium. In some embodiments, R₁₄ is F, Cl, Br, I, —CCl₃, or —CF₃. In some embodiments, R₁₄ is —CH₃, —OCH₃, CH₂CH₃, —OCH₂CH₃, —OH, or —NO₂.

In some embodiments, R₁₅ is H. In some embodiments, R₁₅ is alkyl, alkenyl, alkynyl, cycloalkyl, heteroalkyl, or acyl. In some embodiments, R₁₅ is C₁-C₈ alkyl. In some embodiments, R₁₅ is C₁-C₁₅ heteroalkyl. In some embodiments, R₁₅ is methyl, ethyl, isopropyl, or tert-butyl. In some embodiments, R₁₅ is H or methyl.

In some embodiments, R₁₇ is H. In some embodiments, R₁₇ is alkyl, alkenyl, alkynyl, aryl, arylalkyl, arylheteroalkyl, cycloalkyl, heteroalkyl, or heteroaryl. In some embodiments, R₁₇ is C₁-C₈ alkyl. In some embodiments, R₁₇ is a C₁-C₈ alkenyl, alkynyl, aryl, arylalkyl, arylheteroalkyl, cycloalkyl, heteroalkyl, or heteroaryl group. In some embodiments, R₁₇ is heteroalkyl. In some embodiments, R₁₇ is T. In some embodiments, R₁₇ is —[(CH₂CH₂)—O]_{q}—R₁₃. In some embodiments, R₁₇ is R₉C(O)—, R₁₀OC(O)—, or (R₁₁O)(R₁₂O)P(O)—. In some embodiments, R₁₇ is R₉C(O)—. In some embodiments, R₁₇ is CH₃C(O)—. In some embodiments, R₁₇ is T-C(O)—. In some embodiments, R₁₇ is R₁₃[O—(CH₂CH₂)]_{q}—C(O)—. In some embodiments, R₁ is CH₃—O—CH₂CH₂—C(O)—. In some embodiments, R₁₇ is CH₃—O—CH₂CH₂—O—CH₂—C(O)—. In some embodiments R₁₇ is R₁₀OC(O)—. In some embodiments, R₁₇ is CH₃CH₂OC(O)—. In some embodiments, R₁₇ is R₁₃[O—(CH₂CH₂)]_{q}—O—C(O)—. In some embodiments, R₁₇ is CH₃—[O—(CH₂CH₂)]_{q}—O—C(O)—. In some embodiments, R₁₇ is CH₃—[O—(CH₂CH₂)]₇—O—C(O)—. In some embodiments, R₁ is (R₁₁O)(R₁₂O)P(O)—. In some embodiments, R₁₇ is (R₁₃—[O—(CH₂CH₂)]_{q}—O—)(R₁₃—[O—(CH₂CH₂)]_{q}—O—)P(O)—. In some embodiments, R₁₇ is (CH₃—[O—(CH₂CH₂)]_{q}—O—)(CH₃—[O—(CH₂CH₂)]_{q}—O—)P(O)—. In some embodiments, R₁₇ is (CH₃—[O—(CH₂CH₂)]7-O)(CH₃—[O—(CH₂CH₂)]₇—O)P(O)—. In some embodiments, R₁₇ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O). In some embodiments, R₁₇ is R₁₁R₁₂NC(O)—. In some embodiments, R₁ is R₁₀S(O)—. In some embodiments, R₁₇ is R₁₀S(O)₂—. In some embodiments, R₁₇ is R₁₀OS(O)—. In some embodiments, R₁₇ is R₁₀OS(O)₂—. In some embodiments, R₁₇ is R₁₁R₁₂N(R₉O)P(O). In some embodiments, R₁₇ is not Cbz, Boc, Bpoc, Nps, Ddz, Fmoc, ivDde, Msc, Nsc, Bsmoc, Sps, or Esc.

In some embodiments, n is 1, 2, 3, or 4. In some embodiments, n is 5 or 6. In some embodiments, n is 1. In some embodiments, n is 2. In some embodiments, n is 3. In some embodiments, n is 4. In some embodiments, n is 5. In some embodiments, n is 6.

In some embodiments, m is 1, 2, 3, or 4. In some embodiments, m is 5 or 6. In some embodiments, m is 1. In some embodiments, m is 2. In some embodiments, m is 3. In some embodiments, m is 4. In some embodiments, m is 5. In some embodiments, m is 6.

In some embodiments, p is 1. In some embodiments, p is 2. In some embodiments, p is 3. In some embodiments, p is 4. In some embodiments, p is 5.

In some embodiments, q is 1-20. In some embodiments, q is 5-20. In some embodiments, q is 1-20. In some embodiments, q is 1-15. In some embodiments, q is 5-15. In some embodiments, q is 10-15. In some embodiments, q is 20. In some embodiments, q is 13. In some embodiments, q is 7.

In some embodiments, x is 0. In some embodiments, x is 1. In some embodiments, x is 1. In some embodiments, w is 0. In some embodiments, w is 1. In some embodiments, w is 2. In some embodiments, x is 0 and w is 0.

In some embodiments, the stereochemistry at the carbon atom labeled *4 is D. In some embodiments, the stereochemistry at the carbon atom labeled *4 is L. In some embodiments, the stereochemistry at the carbon atom labeled *3 is D. In some embodiments, the stereochemistry at the carbon atom labeled *3 is L. In some embodiments, the stereochemistry at the carbon atom labeled *2 is D. In some embodiments, the stereochemistry at the carbon atom labeled *2 is L. In some embodiments, the stereochemistry at the carbon atom labeled 0.1 is D. In some embodiments, the stereochemistry at the carbon atom labeled *1 is L.

In some embodiments, the stereochemistry at the carbon atom labeled *4 is D, the stereochemistry at the carbon atom labeled *3 is L, the stereochemistry at the carbon atom labeled *2 is L, and the stereochemistry at the carbon atom labeled *1 is L. In some embodiments, the stereochemistry at the carbon atom labeled *4 is L, the stereochemistry at the carbon atom labeled *3 is D, the stereochemistry at the carbon atom labeled *2 is D, and the stereochemistry at the carbon atom labeled 0.1 is D. In some embodiments, the stereochemistry at the carbon atom labeled *4 is D, the stereochemistry at the carbon atom labeled *3 is D, the stereochemistry at the carbon atom labeled *2 is D, and the stereochemistry at the carbon atom labeled 0.1 is D. In some embodiments, the stereochemistry at the carbon atom labeled *4 is L, the stereochemistry at the carbon atom labeled *3 is L, the stereochemistry at the carbon atom labeled *2 is L, and the stereochemistry at the carbon atom labeled 0.1 is L. In some embodiments, the stereochemistry at the carbon atom labeled *4 is D, the stereochemistry at the carbon atom labeled *3 is L, the stereochemistry at the carbon atom labeled *2 is D, and the stereochemistry at the carbon atom labeled 0.1 is L. In some embodiments, the stereochemistry at the carbon atom labeled *4 is L, the stereochemistry at the carbon atom labeled *3 is D, the stereochemistry at the carbon atom labeled *2 is L, and the stereochemistry at the carbon atom labeled *1 is D.

In some embodiments, the compound is

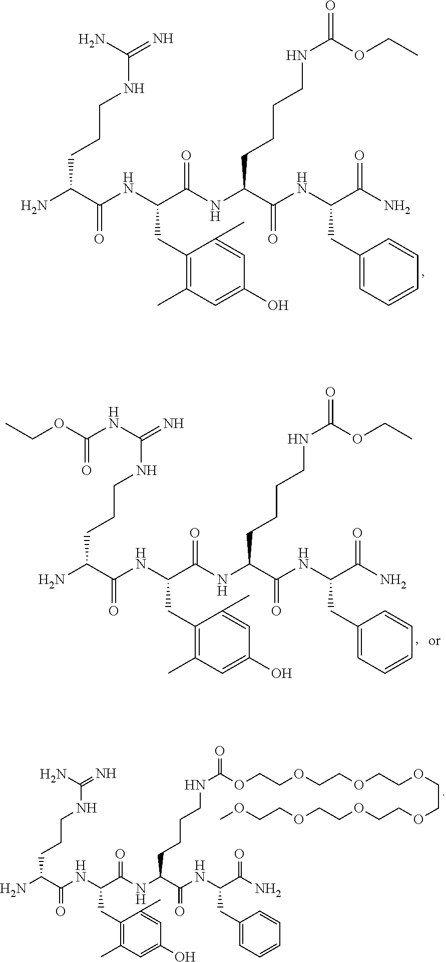 [SMILES: CCOC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O ; CCOC(=O)NCCCC[C@H](NC(=O)[C@H](Cc1c(C)cc(O)cc1C)NC(=O)[C@H](N)CCCNC(=N)NC(=O)OCC)C(=O)N[C@@H](Cc1ccccc1)C(N)=O ; COCCOCCOCCOCCOCCOCCOCCOC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O]

In some embodiments, the compound is

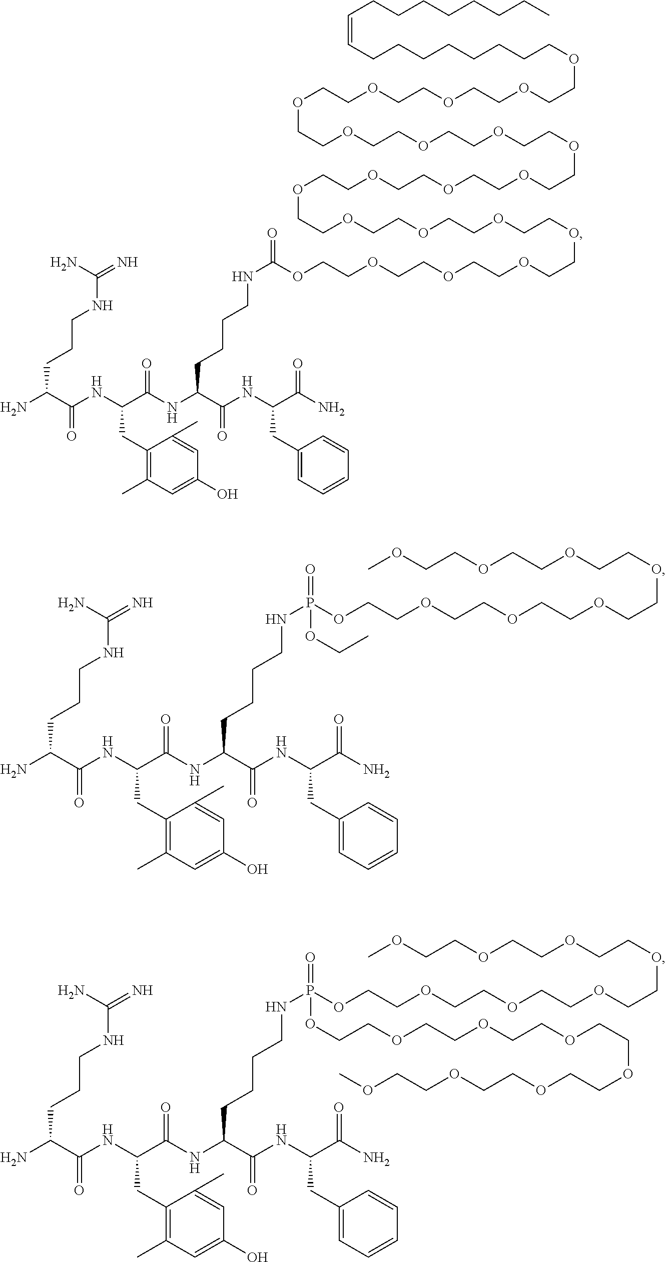 [SMILES: CCCCCCCC/C=C\CCCCCCCCOCCOCCOCCOCCOCCOCCOCCOCCOCCOCCOCCOCCOCCOCCOCCOCCOCCOCCOCCOCCOC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O ; CCOP(=O)(NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O)OCCOCCOCCOCCOCCOCCOCCOC ; COCCOCCOCCOCCOCCOCCOCCOP(=O)(NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O)OCCOCCOCCOCCOCCOCCOCCOC]

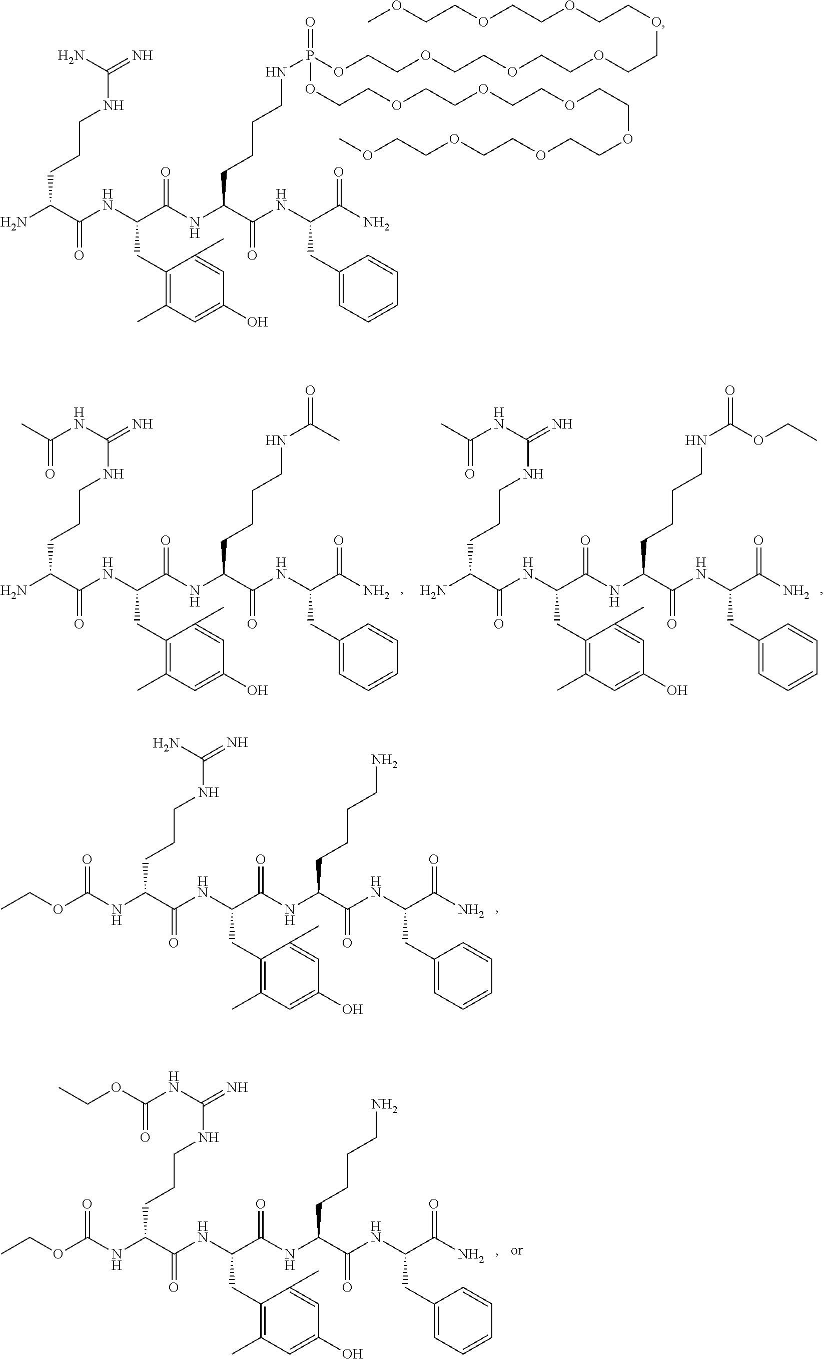

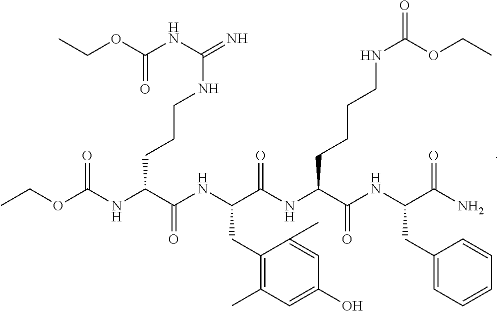 [SMILES: CCOC(=O)NCCCC[C@H](NC(=O)[C@H](Cc1c(C)cc(O)cc1C)NC(=O)[C@@H](CCCNC(=N)NC(=O)OCC)NC(=O)OCC)C(=O)N[C@@H](Cc1ccccc1)C(N)=O]

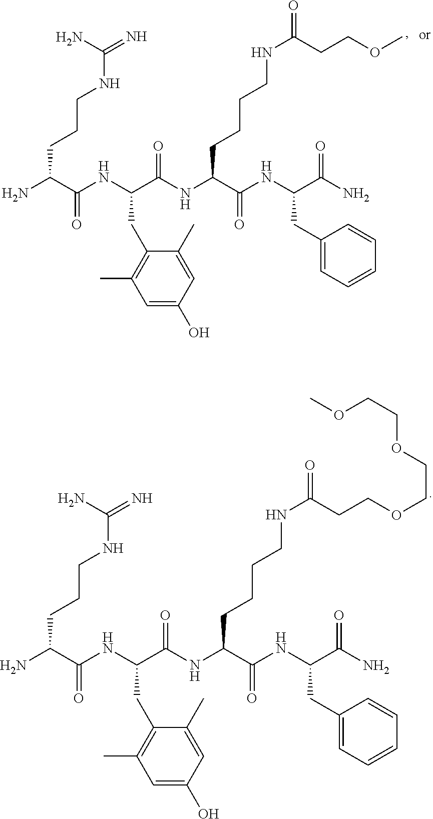 [SMILES: COCCC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O ; COCCOCCOCCC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O]

In some embodiments, the compound is

In some embodiments, the invention provides compounds of Formula (II)

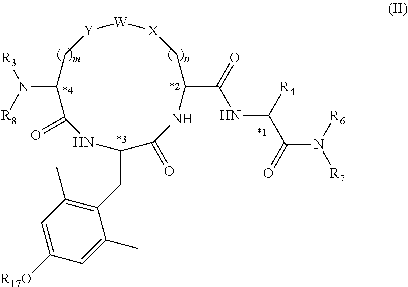

wherein:

X is —N(R₁₅)—,

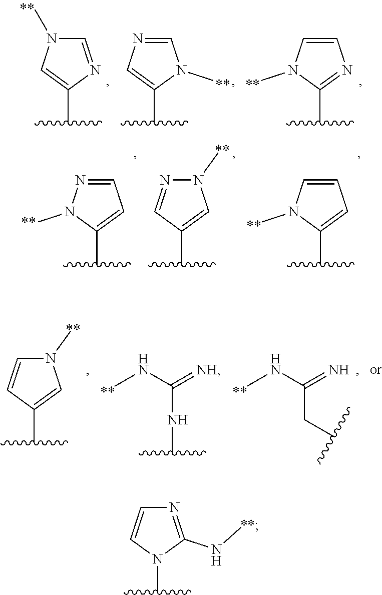

Y is —N(R₁₅)—,

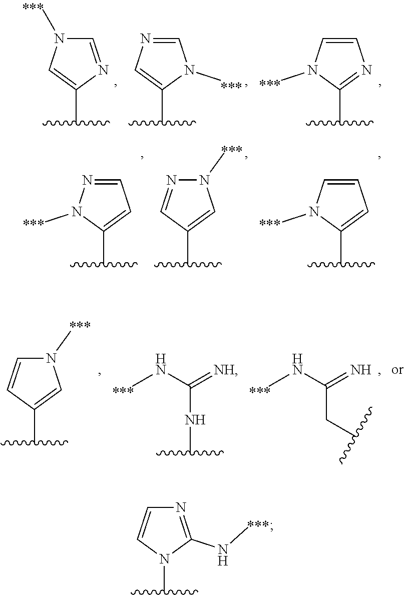

W is —C(O)—, —C(S)—, —C(R₁₆)₂—, —S(O)—, —S(O₂)—, or —P(O)[Q(R₁₀)]—;
Q is O or a bond;

R₃ and R₁₇ are independently H, alkyl, alkenyl, alkynyl, aryl, arylalkyl, arylheteroalkyl, cycloalkyl, heteroalkyl, heteroaryl, T, R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₁O)(R₁₂O)P(O)—, or R₁₁R₁₂N(R₉O)P(O)—;

R₄ is alkyl, cycloalkyl, aryl, arylalkyl, heteroaryl, arylheteroalkyl, T, a side-chain of a naturally or non-naturally occurring chiral amino acid,

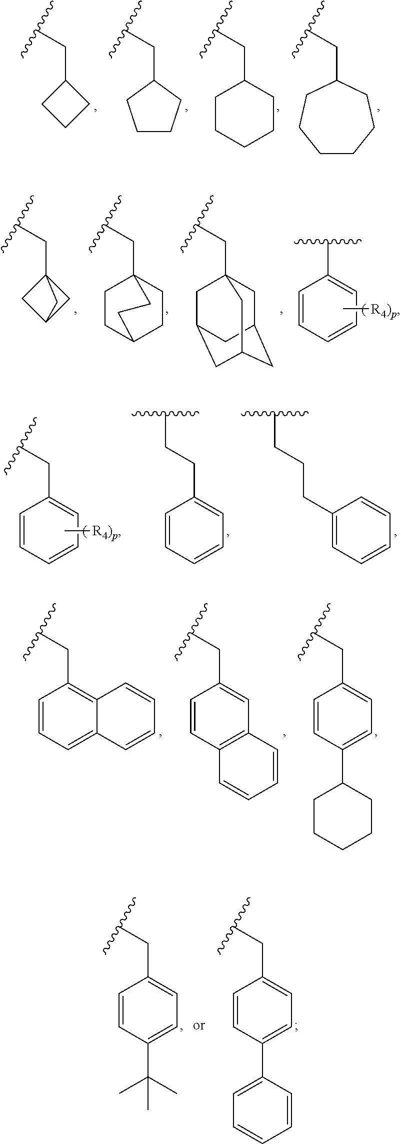

R₆ and R₇ are independently H, alkyl, or acyl; or R₆ and R₇ together with the nitrogen atom to which they are attached form a 4-6-membered heterocyclic ring;

R₈ is H, alkyl, heteroalkyl, or acyl;

R₉, R₁₁, and R₁₂ are independently H, alkyl, alkenyl, alkynyl, heteroalkyl, cycloalkyl, aryl, arylalkyl, heteroaryl, arylheteroalkyl, heteroarylheteroalkyl, or T;

R₁₁ and R₁₂ can be taken together to form a heterocyclic ring;

R₁₀ is alkyl, alkenyl, alkynyl, heteroalkyl, cycloalkyl, aryl, arylalkyl, heteroaryl, arylheteroalkyl, heteroarylheteroalkyl or T;

R₁₃ is H, methyl, ethyl, isopropyl or tert-butyl;

R₁₄ is independently D, F, Cl, Br, I, —CH₃, —OCH₃, CH₂CH₃, —OCH₂CH₃, —CCl₃, —CF₃, —OH, or —NO₂;

R₁₅ is H, alkyl, alkenyl, alkynyl, cycloalkyl, heteroalkyl, or acyl;

R₁₆ is alkyl, alkenyl, alkynyl, heteroalkyl, cycloalkyl, aryl, or arylalkyl;

T is —(CH₂)_{w}—(O)ₓ—[(CH₂CH₂)—O]_{q}—R₁₃;

the absolute stereochemistry at each of stereocenters *1, *2, *3 and *4 is independently

R (D for an amino acid) or S (L for an amino acid);

n and m are independently 1, 2, 3, 4, 5, or 6;

p is 0, 1, 2, 3, 4, or 5;

q is an integer from 1-30 inclusive;

x is 0 or 1; and

w is 0, 1 or 2; provided that: if x is 0, then w is 0; and if w is 0, then y is 0;

“**” denotes the point of attachment of X to W; and

“***” denotes the point of attachment of W to Y.

In some embodiments, X is —N(R₁₅)—. In some embodiments, X is

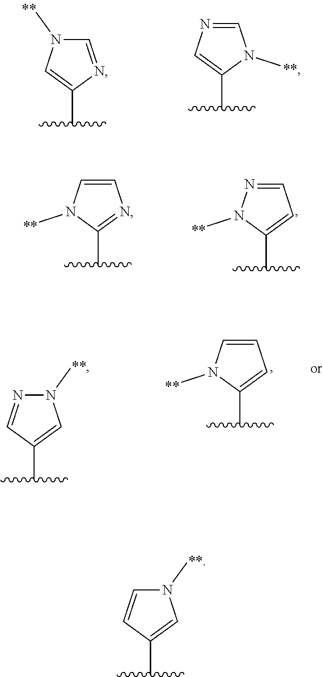

In some embodiments, X is

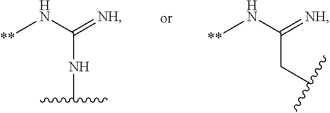

In some embodiments, X is

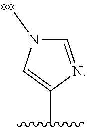 [SMILES: *c1cn(C)cn1]

In some embodiments, X is

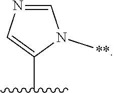 [SMILES: *c1cncn1C]

In some embodiments, X is

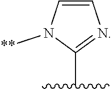 [SMILES: *c1nccn1C]

In some embodiments, X is

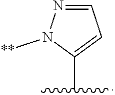 [SMILES: *c1ccnn1C]

In some embodiments, X is

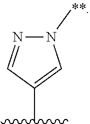 [SMILES: *c1cnn(C)c1]

In some embodiments, X is

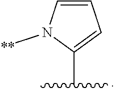 [SMILES: *c1cccn1C]

In some embodiments, X is

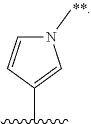 [SMILES: *c1ccn(C)c1]

In some embodiments, X is

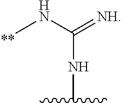 [SMILES: *NC(=N)NC]

In some embodiments, X is

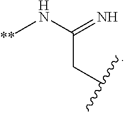 [SMILES: *CC(=N)NC]

In some embodiments, X is

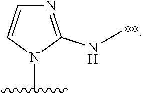 [SMILES: *n1ccnc1NC]

In some embodiments, Y is —N(R₁₅)—. In some embodiments, Y is

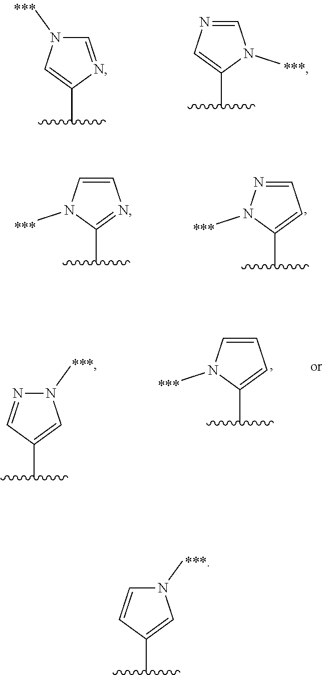

In some embodiments, Y is

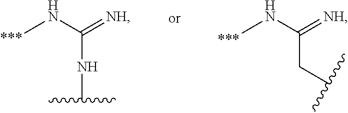

In some embodiments, Y is

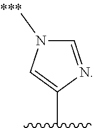 [SMILES: *c1cn(C)cn1]

In some embodiments, Y is

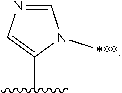 [SMILES: *c1cncn1C]

In some embodiments, Y is

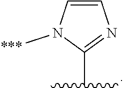 [SMILES: *c1nccn1C]

In some embodiments, Y is

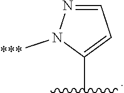 [SMILES: *c1ccnn1C]

In some embodiments, Y is

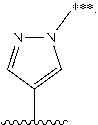 [SMILES: *c1cnn(C)c1]

In some embodiments, Y is

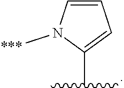 [SMILES: *c1cccn1C]

In some embodiments, Y is

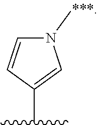 [SMILES: *c1ccn(C)c1]

In some embodiments, Y is

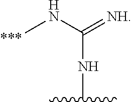 [SMILES: *NC(=N)NC]

In some embodiments, Y is

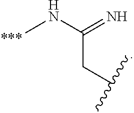 [SMILES: *CC(=N)NC]

In some embodiments, Y is

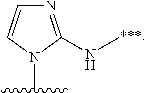 [SMILES: *n1ccnc1NC]

In some embodiments, W is —C(O)—. In some embodiments, W is —C(S)—, or —C(R₁₆)₂—. In some embodiments, W is —S(O)—, or —S(O)₂—. In some embodiments, W is —C(S)—. In some embodiments, W is —C(R₁₆)₂—. In some embodiments, W is —S(O)—. In some embodiments, W is —S(O)₂—. In some embodiments, W is —P(O)[Q(R₁₀)]—;

In some embodiments, Q is O. In some embodiments, Q is a bond.

In some embodiments, R₃ is H. In some embodiments, R₃ is alkyl, alkenyl, alkynyl, aryl, arylalkyl, arylheteroalkyl, cycloalkyl, heteroalkyl, or heteroaryl. In some embodiments, R₃ is C₁-C₈ alkyl. In some embodiments, R₃ is a C₁-C₈ alkenyl, alkynyl, aryl, arylalkyl, arylheteroalkyl, cycloalkyl, heteroalkyl, or heteroaryl group. In some embodiments, R₃ is heteroalkyl. In some embodiments, R₃ is T. In some embodiments, R₃ is —[(CH₂CH₂)—O]_{q}—R₁₃. In some embodiments, R₃ is R₉C(O)—, R₁₀OC(O)—, or (R₁₁O)(R₁₂O)P(O)—. In some embodiments, R₃ is R₉C(O)—. In some embodiments, R₃ is CH₃C(O)—. In some embodiments, R₃ is T-C(O)—. In some embodiments, R₃ is R₁₃—[O—(CH₂CH₂)]_{q}—C(O)—. In some embodiments, R₃ is CH₃—O—CH₂CH₂—C(O)—. In some embodiments, R₃ is CH₃—O—CH₂CH₂—O—CH₂—C(O)—. In some embodiments R₃ is R₁₀OC(O)—. In some embodiments, R₃ is CH₃CH₂OC(O)—. In some embodiments, R₃ is R₁₃—[O—(CH₂CH₂)]_{q}—O—C(O)—. In some embodiments, R₃ is CH₃[O—(CH₂CH₂)]_{q}—O—C(O)—. In some embodiments, R₃ is CH₃-[O —(CH₂CH₂)]7-O—C(O)—. In some embodiments, R₃ is (R₁₁O)(R₁₂O)P(O)—. In some embodiments, R₃ is (R₁₃—[O—(CH₂CH₂)]_{q}—O—)(R₁₃—[O—(CH₂CH₂)]_{q}—O—)P(O)—. In some embodiments, R₃ is (CH₃—[O—(CH₂CH₂)]_{q}—O—)(CH₃—[O—(CH₂CH₂)]_{q}—O—)P(O)—. In some embodiments, R₃ is (CH₃—[O—(CH₂CH₂)]₇—O)(CH₃—[O—(CH₂CH₂)]₇—O)P(O)—. In some embodiments, R₃ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O). In some embodiments, R₃ is R₁₁R₁₂NC(O)—. In some embodiments, R₃ is R₁₀S(O)—. In some embodiments, R₃ is R₁₀S(O)₂—. In some embodiments, R₃ is R₁₀OS(O)—. In some embodiments, R₃ is R₁₀OS(O)₂—. In some embodiments, R₃ is R₁₁R₁₂N(R₉O)P(O). In some embodiments, R₃ is not Cbz, Boc, Bpoc, Nps, Ddz, Fmoc, ivDde, Msc, Nsc, Bsmoc, Sps, or Esc.

In some embodiments, R₄ is alkyl, cycloalkyl, aryl, arylalkyl, heteroaryl, or arylheteroalkyl. In some embodiments, R₄ is T. In some embodiments, R₄ is a side-chain of a naturally or non-naturally occurring chiral amino acid. In some embodiments, R₄ is

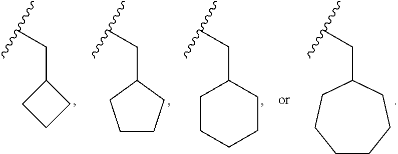

In some embodiments, R₄ is

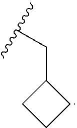 [SMILES: *CC1CCC1]

In some embodiments, R₄ is

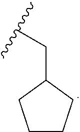 [SMILES: *CC1CCCC1]

In some embodiments, R₄ is

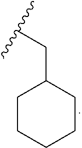 [SMILES: *CC1CCCCC1]

In some embodiments, R₄ is

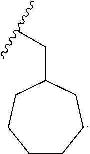 [SMILES: *CC1CCCCCC1]

In some embodiments, R₄ is

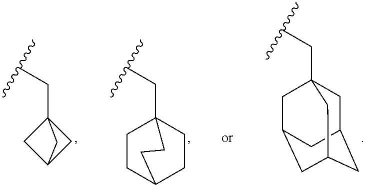 [SMILES: *CC12CC(C1)C2 ; *CC12CCC(CC1)CC2 ; *CC12CC3CC(CC(C3)C1)C2]

In some embodiments, R₄ is

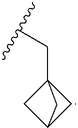 [SMILES: *CC12CC(C1)C2]

In some embodiments, R₄ is

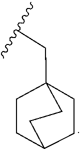 [SMILES: *CC12CCC(CC1)CC2]

In some embodiments, R₄ is

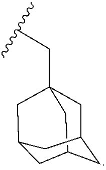 [SMILES: *CC12CC3CC(CC(C3)C1)C2]

In some embodiments, R₄ is

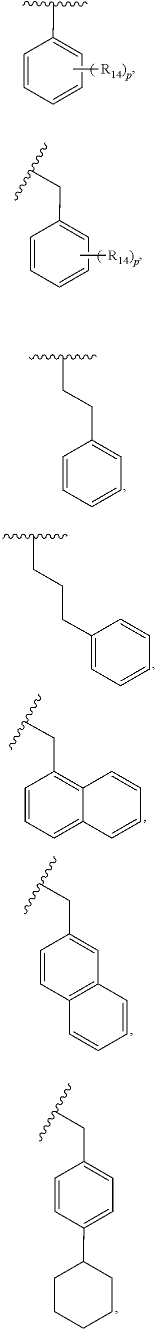

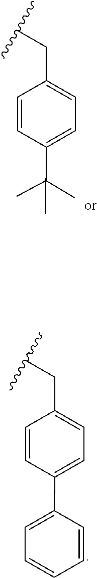 [SMILES: *Cc1ccc(C(C)(C)C)cc1 ; *Cc1ccc(-c2ccccc2)cc1]

In some embodiments, R₄ is

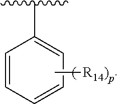

In some embodiments, R₄ is

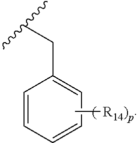

In some embodiments, R₄ is

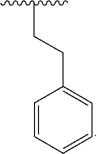 [SMILES: *CCc1ccccc1]

In some embodiments, R₄ is

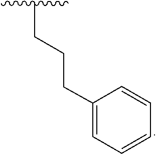 [SMILES: *CCCc1ccccc1]

In some embodiments, R₄ is

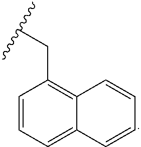 [SMILES: *Cc1cccc2ccccc12]

In some embodiments, R₄ is

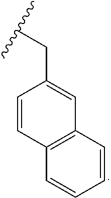 [SMILES: *Cc1ccc2ccccc2c1]

In some embodiments, R₄ is

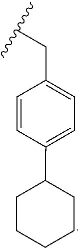 [SMILES: *Cc1ccc(C2CCCCC2)cc1]

In some embodiments, R₄ is

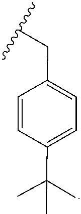 [SMILES: *Cc1ccc(C(C)(C)C)cc1]

In some embodiments, R₄ is

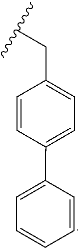 [SMILES: *Cc1ccc(-c2ccccc2)cc1]

In some embodiments, R₄ is —R₁₃, —OR₁₃ or

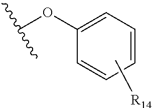

In some embodiments, R₄ is —R₁₃. In some embodiments, R₄ is —OR₁₃. In some embodiments, R₄ is

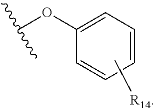

In some embodiments, R₄ is

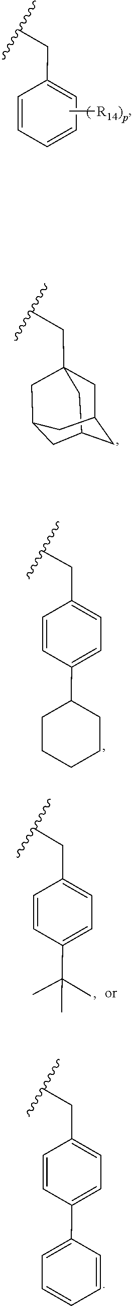

In some embodiments, R₄ is

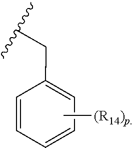

In some embodiments, R₄ is

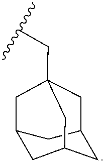 [SMILES: *CC12CC3CC(CC(C3)C1)C2]

In some embodiments, R₄ is

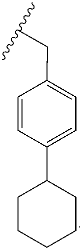 [SMILES: *Cc1ccc(C2CCCCC2)cc1]

In some embodiments, R₄ is

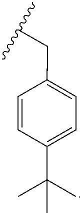 [SMILES: *Cc1ccc(C(C)(C)C)cc1]

In some embodiments, R₄ is

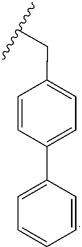 [SMILES: *Cc1ccc(-c2ccccc2)cc1]

In some embodiments, R₆ is H. In some embodiments, R₆ is alkyl. In some embodiments, R₆ is C₁-C₈ alkyl. In some embodiments, R₆ is H, methyl or ethyl. In some embodiments, R₆ is acyl.

In some embodiments, R₇ is H. In some embodiments, R₇ is alkyl. In some embodiments, R₇ is C₁-C₈ alkyl. In some embodiments, R₇ is H, methyl or ethyl. In some embodiments, R₇ is acyl.

In some embodiments, R₆ and R₇ together with the nitrogen atom to which they are attached form a 4-6-membered heterocyclic ring;

In some embodiments, R₈ is H. In some embodiments, R₈ is alkyl, heteroalkyl, or acyl. In some embodiments, R₈ is C₁-C₈ alkyl. In some embodiments, R₈ is C₁-C₁₅ heteroalkyl. In some embodiments, R₈ is H, methyl or ethyl.

In some embodiments, R₉ is H. some embodiments, R₉ is alkyl, alkenyl, alkynyl, heteroalkyl, cycloalkyl, aryl, arylalkyl, heteroaryl, arylheteroalkyl, or heteroarylheteroalkyl. In some embodiments, R₉ is C₁-C₈ alkyl. In some embodiments, R₉ is C₁-C₁₅ heteroalkyl. In some embodiments, R₉ is T. In some embodiments, R₉ is —[(CH₂CH₂)—O]_{q}—R₁₃ and q is 1-20.

In some embodiments, R₁₀ is alkyl, alkenyl, alkynyl, heteroalkyl, cycloalkyl, aryl, arylalkyl, heteroaryl, arylheteroalkyl, or heteroarylheteroalkyl. In some embodiments, R₁₀ is C₁-C₈ alkyl. In some embodiments, R₁₀ is C₁-C₁₅ heteroalkyl. In some embodiments, R₁₀ is T. In some embodiments, R₁₀ is —[(CH₂CH₂)—O]_{q}—R₁₃ and q is 1-20.

In some embodiments, R₁₁ is H. In some embodiments, R₁₁ is alkyl, alkenyl, alkynyl, heteroalkyl, cycloalkyl, aryl, arylalkyl, heteroaryl, arylheteroalkyl, or heteroarylheteroalkyl. In some embodiments, R₁₁ is C₁-C₈ alkyl. In some embodiments, R₁₁ is C₁-C₁₅ heteroalkyl. In some embodiments, R₁₁ is T. In some embodiments, R₁₁ is —[(CH₂CH₂)—O]_{q}—R₁₃ and q is 1-20.

In some embodiments, R₁₂ is H. In some embodiments, R₁₂ is alkyl, alkenyl, alkynyl, heteroalkyl, cycloalkyl, aryl, arylalkyl, heteroaryl, arylheteroalkyl, or heteroarylheteroalkyl. In some embodiments, R₁₂ is C₁-C₈ alkyl. In some embodiments, R₁₂ is C₁-C₁₅ heteroalkyl. In some embodiments, R₁₂ is T. In some embodiments, R₁₂ is —[(CH₂CH₂)—O]_{q}—R₁₃ and q is 1-20.

In some embodiments, R₁₁ and R₁₂ are taken together to form a heterocyclic ring. In some embodiments, the heterocyclic ring is a 3-membered to 7-membered ring. The heterocyclic ring can be substituted or unsubstituted.

In some embodiments, R₁₃ is H. In some embodiments, R₁₃ is methyl, ethyl, isopropyl or tert-butyl.

In some embodiments, R₁₄ is deuterium. In some embodiments, R₁₄ is F, Cl, Br, I, —CCl₃, or —CF₃. In some embodiments, R₁₄ is —CH₃, —OCH₃, CH₂CH₃, —OCH₂CH₃, —OH, or —NO₂.

In some embodiments, R₁₅ is H. In some embodiments, R₁₅ is alkyl, alkenyl, alkynyl, cycloalkyl, heteroalkyl, or acyl. In some embodiments, R₁₅ is C₁-C₈ alkyl. In some embodiments, R₁₅ is C₁-C₁₅ heteroalkyl. In some embodiments, R₁₅ is methyl, ethyl, isopropyl, or tert-butyl. In some embodiments, R₁₅ is H or methyl.

In some embodiments, R₁₆ is alkyl. In some embodiments, R₁₆ is alkenyl. In some embodiments, R₁₆ is alkynyl. In some embodiments, R₁₆ is heteroalkyl. In some embodiments, R₁₆ is cycloalkyl. In some embodiments, R₁₆ is aryl. In some embodiments, R₁₆ is arylalkyl

In some embodiments, R₁₇ is H. In some embodiments, R₁₇ is alkyl, alkenyl, alkynyl, aryl, arylalkyl, arylheteroalkyl, cycloalkyl, heteroalkyl, or heteroaryl. In some embodiments, R₄ is methyl or ethyl. In some embodiments, R₁₇ is —(CH₂)—(O)—[(CH₂CH₂)—O]_{q}—R₁₃ or —(CH₂)₂—(O)—[(CH₂CH₂)—O]_{q}—R₁₃. In some embodiments, R₄ is R₉C(O)—, R₁₀OC(O)—, or (R₁₁O)(R₁₂O)P(O)—. In some embodiments, R₁₇ is R₉C(O)—, R₁₀OC(O)—, or (R₁₁O)(R₁₂O)P(O)—. In some embodiments, R₁₇ is R₉C(O)—. In some embodiments, R₁₇ is R₁₀OC(O)—. In some embodiments, R₁₇ is (R₁₁O)(R₁₂₀)P(O)—. In some embodiments, R₁₇ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—. In some embodiments, R₁₇ is R₁₁R₁₂NC(O)—. In some embodiments, R₁₇ is R₁₀S(O)—. In some embodiments, R₁₇ is R₁₀S(O)₂—. In some embodiments, R₁₇ is R₁₀OS(O)—. In some embodiments, R₁₇ is R₁₀OS(O)₂—. In some embodiments, R₁₇ is R₁₁R₁₂N(R₉O)P(O)—.

In some embodiments, n is 1, 2, 3, or 4. In some embodiments, n is 5 or 6. In some embodiments, n is 1. In some embodiments, n is 2. In some embodiments, n is 3. In some embodiments, n is 4. In some embodiments, n is 5. In some embodiments, n is 6.

In some embodiments, m is 1, 2, 3, or 4. In some embodiments, m is 5 or 6. In some embodiments, m is 1. In some embodiments, m is 2. In some embodiments, m is 3. In some embodiments, m is 4. In some embodiments, m is 5. In some embodiments, m is 6.

In some embodiments, p is 1. In some embodiments, p is 2. In some embodiments, p is 3. In some embodiments, p is 4. In some embodiments, p is 5.

In some embodiments, q is 1-20. In some embodiments, q is 5-20. In some embodiments, q is 1-20. In some embodiments, q is 1-15. In some embodiments, q is 5-15. In some embodiments, q is 10-15. In some embodiments, q is 20. In some embodiments, q is 13. In some embodiments, q is 7.

In some embodiments, x is 0. In some embodiments, x is 1. In some embodiments, x is 1. In some embodiments, w is 0. In some embodiments, w is 1. In some embodiments, w is 2. In some embodiments, x is 0 and w is 0.

In some embodiments, the stereochemistry at the carbon atom labeled *4 is D. In some embodiments, the stereochemistry at the carbon atom labeled *4 is L. In some embodiments, the stereochemistry at the carbon atom labeled *3 is D. In some embodiments, the stereochemistry at the carbon atom labeled *3 is L. In some embodiments, the stereochemistry at the carbon atom labeled *2 is D. In some embodiments, the stereochemistry at the carbon atom labeled *2 is L. In some embodiments, the stereochemistry at the carbon atom labeled 0.1 is D. In some embodiments, the stereochemistry at the carbon atom labeled *1 is L.

In some embodiments, the stereochemistry at the carbon atom labeled *4 is D, the stereochemistry at the carbon atom labeled *3 is L, the stereochemistry at the carbon atom labeled *2 is L, and the stereochemistry at the carbon atom labeled *1 is L. In some embodiments, the stereochemistry at the carbon atom labeled *4 is L, the stereochemistry at the carbon atom labeled *3 is D, the stereochemistry at the carbon atom labeled *2 is D, and the stereochemistry at the carbon atom labeled 0.1 is D. In some embodiments, the stereochemistry at the carbon atom labeled *4 is D, the stereochemistry at the carbon atom labeled *3 is D, the stereochemistry at the carbon atom labeled *2 is D, and the stereochemistry at the carbon atom labeled 0.1 is D. In some embodiments, the stereochemistry at the carbon atom labeled *4 is L, the stereochemistry at the carbon atom labeled *3 is L, the stereochemistry at the carbon atom labeled *2 is L, and the stereochemistry at the carbon atom labeled 0.1 is L. In some embodiments, the stereochemistry at the carbon atom labeled *4 is D, the stereochemistry at the carbon atom labeled *3 is L, the stereochemistry at the carbon atom labeled *2 is D, and the stereochemistry at the carbon atom labeled 0.1 is L. In some embodiments, the stereochemistry at the carbon atom labeled *4 is L, the stereochemistry at the carbon atom labeled *3 is D, the stereochemistry at the carbon atom labeled *2 is L, and the stereochemistry at the carbon atom labeled *1 is D.

In some embodiments, the compound is

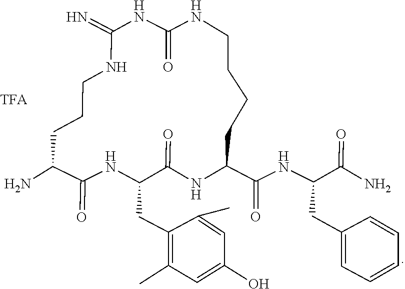 [SMILES: Cc1cc(O)cc(C)c1C[C@@H]1NC(=O)[C@H](N)CCCNC(=N)NC(=O)NCCCC[C@@H](C(=O)N[C@@H](Cc2ccccc2)C(N)=O)NC1=O]

In some embodiments, the compound is

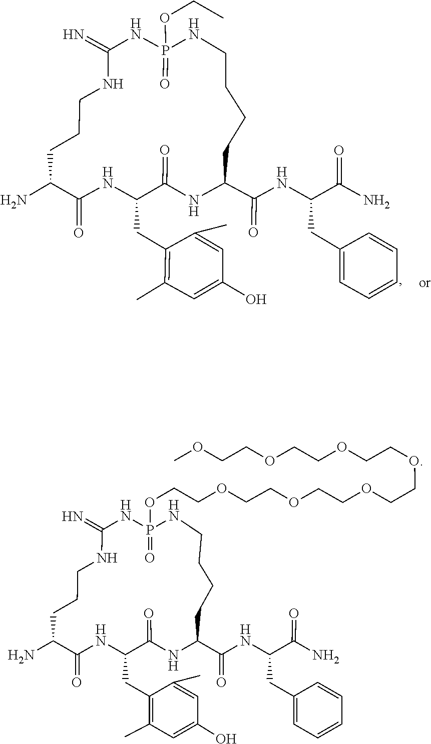 [SMILES: CCOP1(=O)NCCCC[C@@H](C(=O)N[C@@H](Cc2ccccc2)C(N)=O)NC(=O)[C@H](Cc2c(C)cc(O)cc2C)NC(=O)[C@H](N)CCCNC(=N)N1 ; COCCOCCOCCOCCOCCOCCOCCOP1(=O)NCCCC[C@@H](C(=O)N[C@@H](Cc2ccccc2)C(N)=O)NC(=O)[C@H](Cc2c(C)cc(O)cc2C)NC(=O)[C@H](N)CCCNC(=N)N1]

Peptide Synthesis

The peptidic compounds of the invention may be prepared using a peptide synthesis method, such as conventional liquid-phase peptide synthesis or solid-phase peptide synthesis, or by peptide synthesis by means of an automated peptide synthesizer (Kelley et al., Genetics Engineering Principles and Methods, Setlow, J. K. eds., Plenum Press NY. (1990) Vol. 12, pp. 1 to 19; Stewart et al., Solid-Phase Peptide Synthesis (1989) W. H.; Houghten, Proc. Natl. Acad. Sci. USA (1985) 82: p. 5132). The peptide thus produced can be collected or purified by a routine method, for example, chromatography, such as gel filtration chromatography, ion exchange column chromatography, affinity chromatography, reverse phase column chromatography, and HPLC, ammonium sulfate fractionation, ultrafiltration, and immunoadsorption.

In a solid-phase peptide synthesis, peptides are typically synthesized from the carbonyl group side (C-terminus) to amino group side (N-terminus) of the amino acid chain. In certain embodiments, an amino-protected amino acid is covalently bound to a solid support material through the carboxyl group of the amino acid, typically via an ester or amido bond and optionally via a linking group. The amino group may be deprotected and reacted with (i.e., “coupled” with) the carbonyl group of a second amino-protected amino acid using a coupling reagent, yielding a dipeptide bound to a solid support. Typically in solid phase synthesis, after coupling, a capping step is performed to cap (render unreactive) any unreacted amine groups. These steps (i.e., deprotection, coupling, and optionally capping) may be repeated to form the desired peptide chain. Once the desired peptide chain is complete, the peptide may be cleaved from the solid support.

In certain embodiments, the protecting groups used on the amino groups of the amino acid residues include 9-fluorenylmethyloxycarbonyl group (Fmoc) and t-butyloxycarbonyl (Boc). The Fmoc group is removed from the amino terminus with base while the Boc group is removed with acid. In alternative embodiments, the amino protecting group may be formyl, acrylyl (Acr), benzoyl (Bz), acetyl (Ac), trifluoroacetyl, substituted or unsubstituted groups of aralkyloxycarbonyl type, such as the benzyloxycarbonyl (Z, cbz or Cbz), p-chlorobenzyloxycarbonyl, p-bromobenzyloxycarbonyl, p-nitrobenzyloxycarbonyl, p-methoxybenzyloxycarbonyl, benzhydryloxycarbonyl, 2(p-biphenylyl)isopropyloxycarbonyl, 2-(3,5-dimethoxyphenyl)isopropyloxycarbonyl, p-phenylazobenzyloxycarbonyl, triphenylphosphonoethyloxycarbonyl or 9-fluorenylmethyloxycarbonyl group (Fmoc), substituted or unsubstituted groups of alkyloxycarbonyl type, such as the tert-butyloxycarbonyl (BOC), tert-amyloxycarbonyl, diisopropylmethyloxycarbonyl, isopropyloxycarbonyl, ethyloxycarbonyl, allyloxycarbonyl, 2 methyl sulphonylethyloxycarbonyl or 2,2,2-trichloroethyloxycarbonyl group, groups of cycloalkyloxycarbonyl type, such as the cyclopentyloxycarbonyl, cyclohexyloxycarbonyl, adamantyloxycarbonyl or isobornyloxycarbonyl group, and groups containing a hetero atom, such as the benzenesulphonyl, p-toluenesulphonyl, mesitylenesulphonyl, methoxytrimethylphenylsulphonyl, 2-nitrobenzenesulfonyl, 2-nitrobenzenesulfenyl, 4-nitrobenzenesulfonyl or 4-nitrobenzenesulfenyl group.

Many amino acids bear reactive functional groups in the side chain. In certain embodiments, such functional groups are protected in order to prevent the functional groups from reacting with the incoming amino acid. The protecting groups used with these functional groups must be stable to the conditions of peptide synthesis, but may be removed before, after, or concomitantly with cleavage of the peptide from the solid support. Further reference is also made to: Isidro-Llobet, A., Alvarez, M., Albericio, F., “Amino Acid-Protecting Groups”; Chem. Rev., 109: 2455-2504 (2009) as a comprehensive review of protecting groups commonly used in peptide synthesis.

In certain embodiments, the solid support material used in the solid-phase peptide synthesis method is a gel-type support such as polystyrene, polyacrylamide, or polyethylene glycol. Alternatively, materials such as pore glass, cellulose fibers, or polystyrene may be functionalized at their surface to provide a solid support for peptide synthesis.

Coupling reagents that may be used in the solid-phase peptide synthesis described herein are typically carbodiimide reagents. Examples of carbodiimide reagents include, but are not limited to, N,N′-dicyclohexylcarbodiimide (DCC), 1-(3-dimethylaminopropyl)-3-ethylcarbodiimide (EDC), and its HCl salt (EDC·HCl), N-cyclohexyl-N′-isopropylcarbodiimide (CIC), N,N′-diisopropylcarbodiimide (DIC), N-tert-butyl-N′-methylcarbodiimide (BMC), N-tert-butyl-N′-ethylcarbodiimide (BEC), bis[[4-(2,2-dimethyl-1,3-dioxolyl)]-methyl]carbodiimide (BDDC), and N,N-dicyclopentylcarbodiimide. DCC is a preferred coupling reagent. Other coupling agents include HATU and HBTU, generally used in combination with an organic base such as DIEA and a hindered pyridine-type base such as lutidine or collidine.

In some embodiments, the amino acids can be activated toward coupling by forming N-carboxyanhydrides as described in Fuller et al., Urethane-Protected α-Amino Acid N-Carboxyanhydrides and Peptide Synthesis, Biopolymers (Peptide Science), Vol. 40, 183-205 (1996); and WO 2018/034901.

In certain exemplary embodiments, linear compounds 1 are synthesized in a convergent fashion, according to the solid phase synthesis depicted in Scheme 1.

For reference in the following schemes,

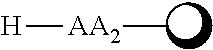

indicates

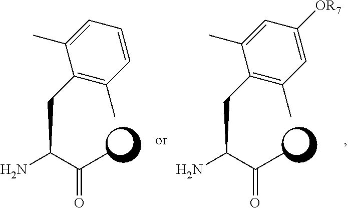

wherein

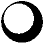

represents a solid support and optionally a linking group.

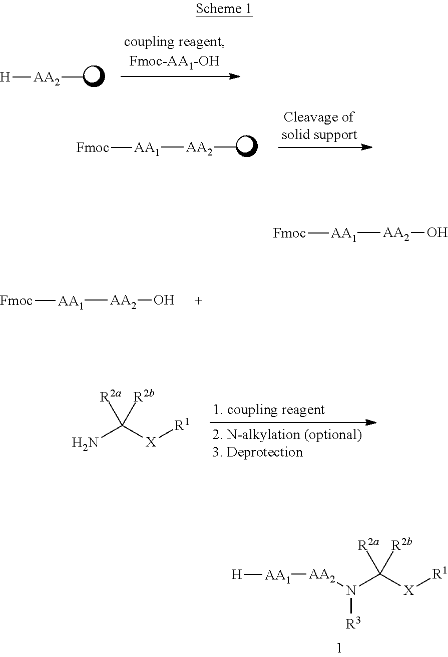

For example, the compound pictured below may be synthesized in such a fashion, as illustrated in Scheme 2.

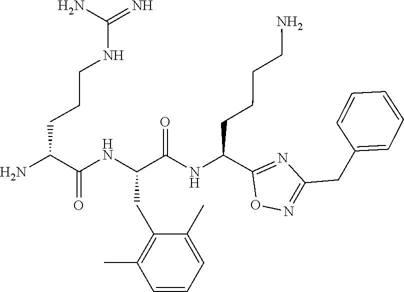 [SMILES: Cc1cccc(C)c1C[C@H](NC(=O)[C@H](N)CCCNC(=N)N)C(=O)N[C@@H](CCCCN)c1nc(Cc2ccccc2)no1]

For reference in the following schemes,

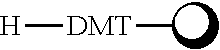

indicates

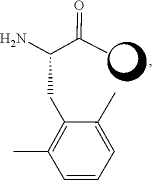

wherein

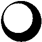

represents a solid support and optionally a linking group.

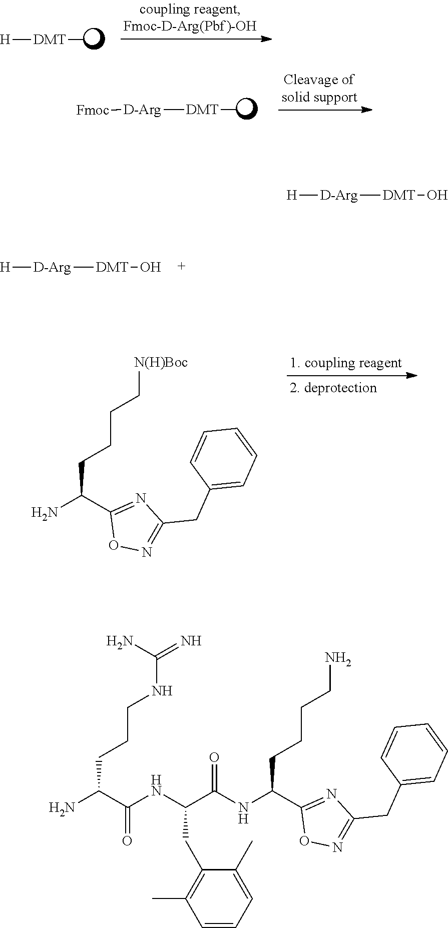

The compounds of the invention (1) may also be synthesized according to conventional liquid-phase peptide synthetic routes, e.g., according to Scheme 3.

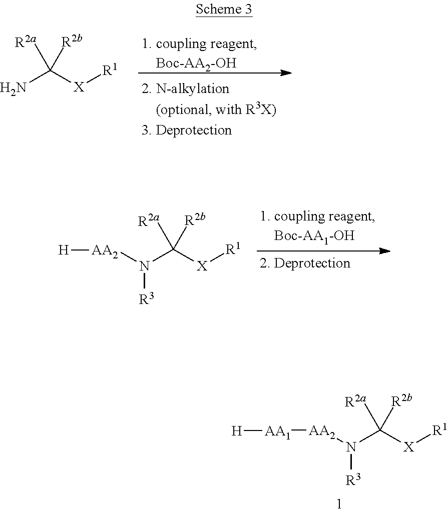

For example, the compound pictured below may be synthesized in such a fashion, as illustrated in Scheme 4.

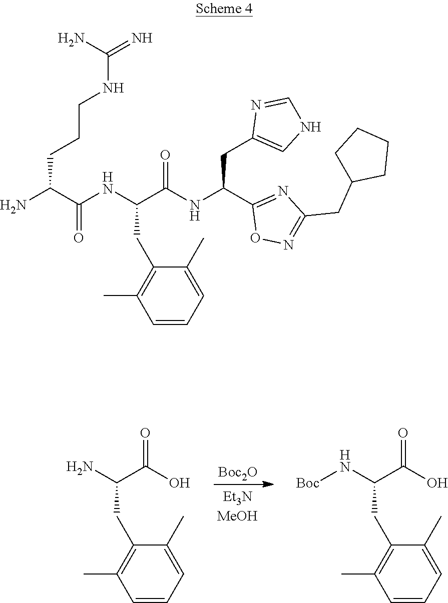

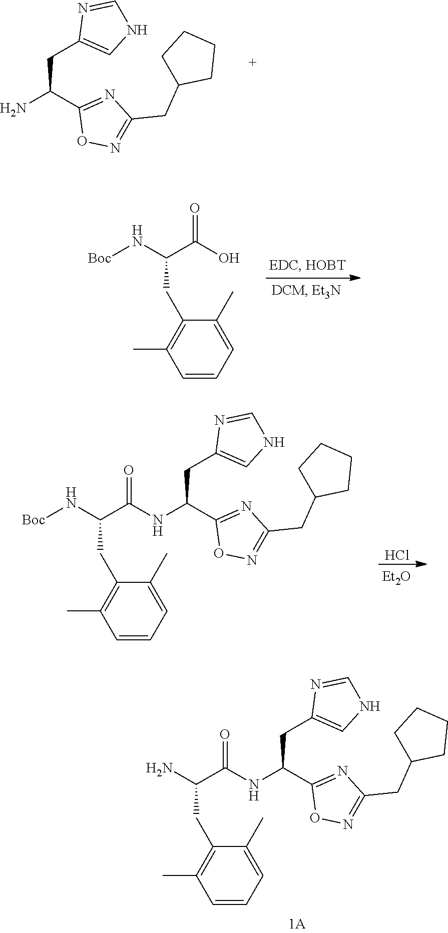

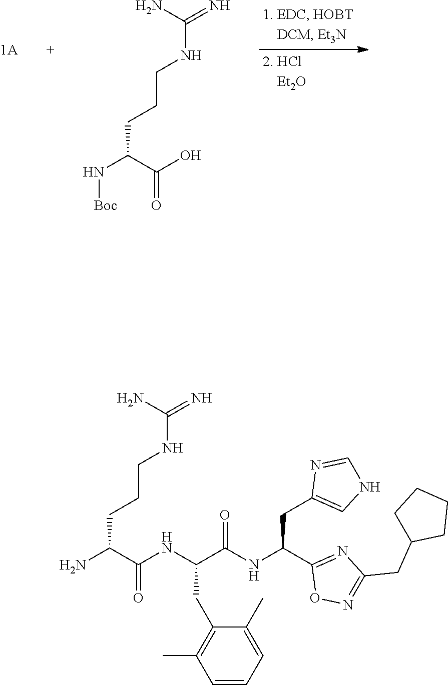 [SMILES: CC(C)(C)OC(=O)N[C@H](CCCNC(=N)N)C(=O)O ; Cc1cccc(C)c1C[C@H](NC(=O)[C@H](N)CCCNC(=N)N)C(=O)N[C@@H](Cc1c[nH]cn1)c1nc(CC2CCCC2)no1]

Elamipretide can be synthesized using NCA-based reagents. Elamipretide may be synthesized by convergent peptide synthesis; e.g., a 2+2 peptide synthesis represented generally by Scheme 5. PG¹-PG⁴ represents protecting groups.

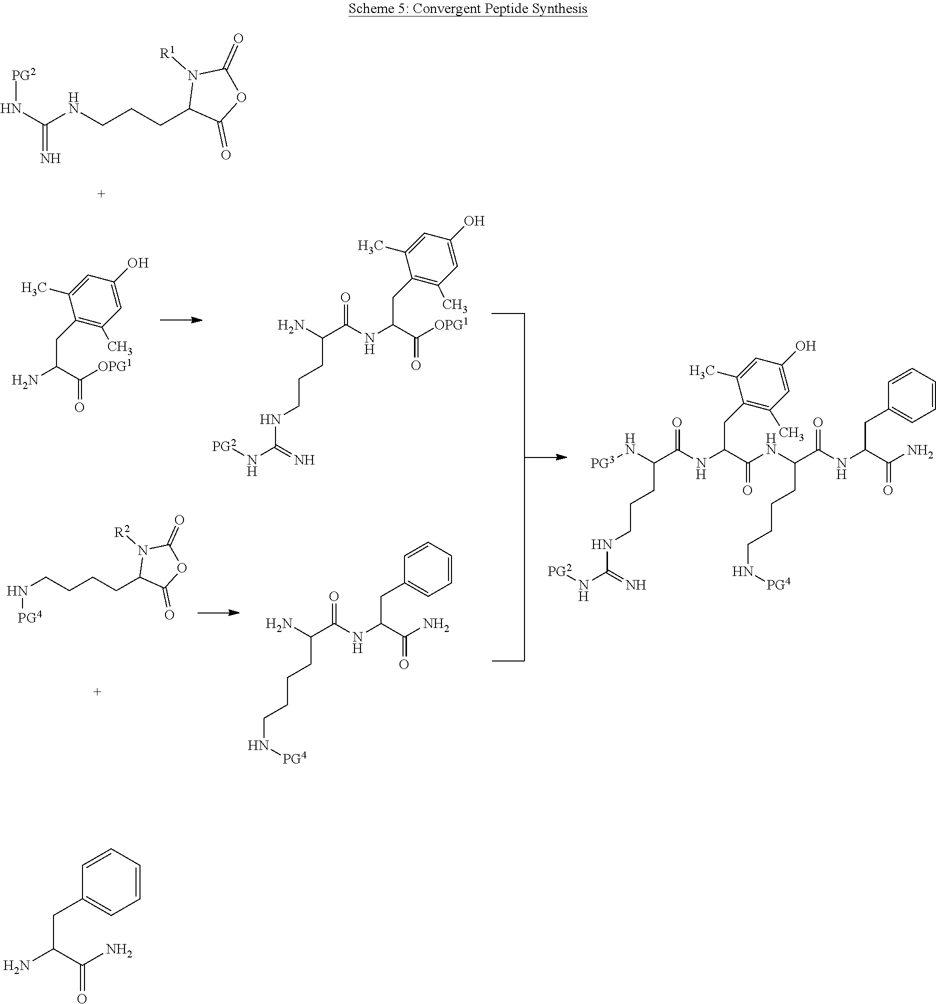

Elamipretide may also be synthesized via a C-to-N linear convergent peptide synthesis, e.g., represented generally by Scheme 6. In such a C-to-N linear peptide synthesis, an NCA reagent is used for each amino acid installation. PG¹-PG⁴ represent protecting groups.

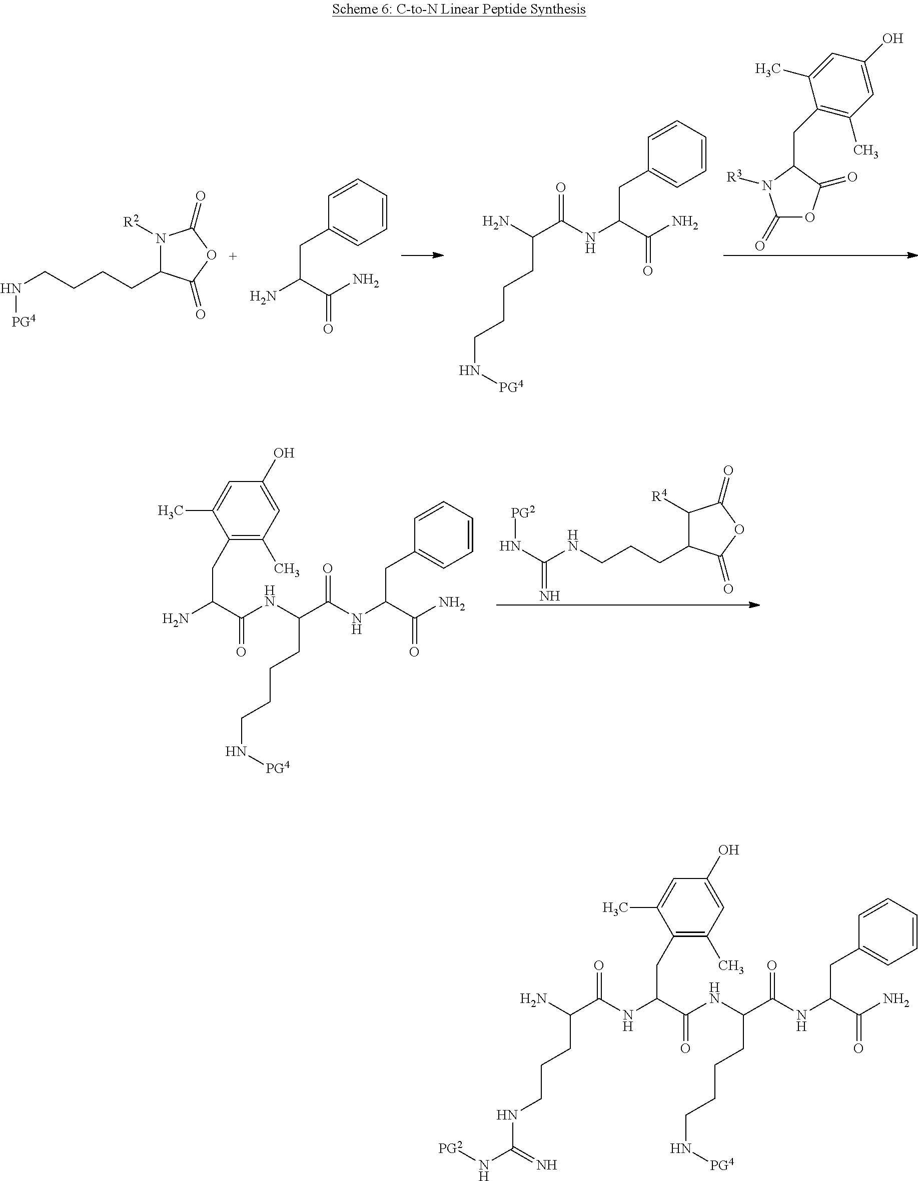

Elamipretide may also be synthesized via alternative linear convergent peptide synthesis routes, such as the route represented generally by Scheme 7. PG¹-PG⁵ represent protecting groups.

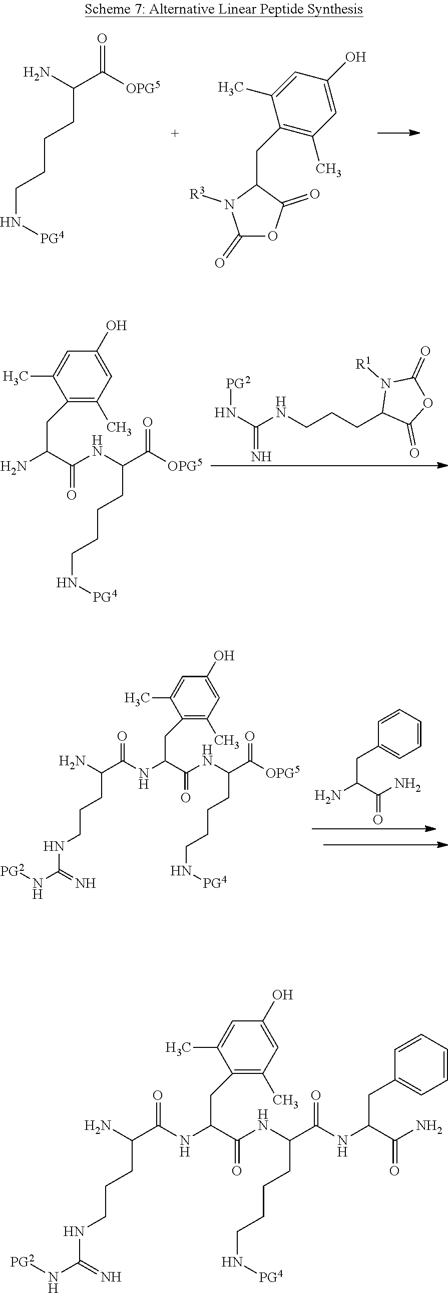

Definitions

| Abbreviation | Compound Name |
|---|---|
| Ac | acetyl |
| ACN or MeCN | acetonitrile |
| AcOH | acetic acid |
| 1-Ada | 1-adamantyl |
| Al | allyl |
| Ala | alanine |
| Alloc | allyloxycarbonyl |
| Arg | arginine |
| Asn | asparagine |
| Asp | aspartic acid |
| Azoc | azidomethyloxycarbonyl |
| 9-BBN | 9-borabicyclo[3.3.1]nonane |
| Bn | benzyl |
| BOC, Boc or t-Boc | tert-butyloxycarbonyl |
| (Boc)₂O or BOC₂O | di-tert-butyl dicarbonate |
| Bom | benzyloxymethyl |
| Bpoc | 2-(4-biphenyl) isopropoxycarbonyl |
| 2-BE | 2-bromoethyl |
| BrBn | 2-bromobenzyl |
| Br | bromine |
| BrPhF | 9-(4-bromophenyl)-9-fluorenyl |
| Br-Z | 2-bromobenzyloxycarbonyl |
| Bsmoc | 1,1-dioxobenzo[b]thiophene-2-ylmethyloxycarbonyl |
| Bum | tert-butoxymethyl |
| Cam | carbamoylmethyl |
| cHx | cyclohexyl |
| Cl | chlorine |
| Cl-Z | 2-chlorobenzyloxycarbonyl |
| Cys | cysteine |
| D | deuterium |
| Dab | diaminobutyric acid |
| Dap | diaminopropionic acid |
| Dcb | 2,6-dichlorobenzyl |
| DCC | N,N-dicyclohexylcarbodiimide |
| DCM | dichloromethane, a.k.a. methylene chloride |
| DCU | N,N-dicyclohexylurea |
| Dde | (1-(4,4-dimethyl-2-6-dioxocyclohex-1-ylidene)-3- |
| | ethyl) |
| Ddz | α,α-dimethyl-3,5-dimethoxybenyloxycarbonyl |
| dio-Fmoc | 2,7-diisooctyl-Fmoc |
| DIAD | diisopropyl azodicarboxylate |
| DIPEA or DIEA | N,N-diisopropylethylamine |
| Dma | 1,1-dimethylallyl |
| Dmab | 4-(N-[1-(4,4-dimethyl-2,6-dioxocyclohexylidene)- |
| | 3-methylbutyl]amino)benzyl |
| DMAP | N,N-dimethyl-4-aminopyridine |
| Dmb | 2,4-dimethoxybenzyl |
| Dmcp | dimethylcyclopropylmethyl |
| DME | 1,2-dimethoxyethane |
| DMF | N,N-dimethylformamide |
| DMT | dimethoxytrityl |
| 2,6-Dmt | 2,6-dimethyltyrosine |
| Dmnb | 4,5-dimethoxy-2-nitrobenzyloxycarbonyl |
| DMSO | dimethylsulfoxide |
| dNBS | 2,4-dinitrobenzenesulfonyl |
| Dnp | 2,4-dinitrophenyl |
| Dnpe | 2-(2,4-dinitrophenyl)ethyl |
| Doc | 2,4-dimethylpent-3-yloxycarbonyl |
| Dts | dithiasuccinoyl |
| DTT | dithiothreitol |
| EDC | 1-ethyl-3-(3-dimethylaminopropyl)carbodiimide |
| Esc | ethanesulfonylethoxycarbonyl |
| Et₃N | triethylamine |
| Et₃O | diethyl ether |
| EtOAc | ethyl acetate |
| EtOH | ethanol |
| F | fluorine |
| Fm | 9-fluorenylmethyl |
| Fmoc | 9-fluorenylmethoxycarbonyl |
| Fmoc(2F) | 2-fluoro-Fmoc |
| Fmoc* | 2,7-di-tert-butyl-Fmoc |
| For | formyl |
| Gln | glutamine |
| Glu | glutamic acid |
| Gly | glycine |
| H | hydrogen |
| HATU | 2-3H-1,2,3 triazolo 4,5-β pyridin-3-yl-1,1,3,3- |
| | tetramethyluronium hexafluorophosphate |
| HBTU | (2-(1/7-benzotriazol-1-yl)-1,1,3,3- |
| | tetramethyluronium hexafluorophosphate |
| HCl | hydrochloric acid |
| His | histidine |
| Hmb | 2-hydroxy-4-methoxybenzyl |
| HMPA | hexamethylphosphoramide |
| Hoc | cyclohexyloxycarbonyl |
| HOAt | 1-Hydroxy-7-azabenzotriazole |
| HOBt | 1-hydroxybenztriazole |
| I | iodine |
| 2-IE | 2-iodoethyl |
| Ile | isoleucine |
| IPA or iPrOH | isopropanol |
| IPAC | isopropyl acetate |
| ivDde | 1-(4,4-dimethyl-2,6-dioxocyclohexyl-1-ylidene)-3- |
| | methylbutyl |
| Leu | leucine |
| Lys | lysine |
| Mbh | 4,4′-dimethyloxybenzhydryl |
| Meb | p-methylbenzyl |
| Men | β-menthyl |
| MeOH | methanol |
| MeSub | 2-methoxy-5-dibenzosuberyl |
| Met | methionine |
| MIM | 1-methyl-3-indolylmethyl |
| Mio-Fmoc | 2-monoisooctyl-Fmoc |
| MIS | 1,2-dimethylindole-3-sulfonyl |
| Mmt | monomethoxytrityl |
| Mob | p-methoxybenzyl |
| Mpe | β-3-methylpent-3-yl |
| Msc | 2-(methylsulfonyl) ethoxycarbonyl |
| MsCl | mesyl chloride or methanesulfonyl chloride |
| MTBE | methyl tert-butyl ether |
| Mtr | 4-methoxy-2,3,6-trimethylphenylsulfonyl |
| Mts | mesitylene-2-sulfonyl |
| Mtt | 4-methyltrityl |
| NMM | N-methylmorpholine |
| NMP | N-methylpyrrolidone |
| NPPOC | 2-(2-nitrophenyl) propyloxycarbonyl |
| Nps | 2-nitrophenylsulfanyl |
| Npyl | 3-nitro-2-pyridinesulfenyl |
| Nsc | 2-(4-nitrophenylsulfonyl) ethoxycarbonyl |
| α-Nsmoc | 1,1-dioxonaphtho[1,2-β] thiophene |
| NVOC | 6-nitroveratryloxycarbonyl |
| oNBS | o-nitrobenzenesulfonyl |
| oNZ | o-nitrobenzyloxycarbonyl |
| Orn | ornithine |
| Pac | phenacyl |
| Pbf | pentamethyl-2,3-dihydrobenzofuran-5-sulfonyl |
| PE | petroleum ether |
| PhAcm | phenylacetamidomethyl |
| Phdec | phenyldithioethyloxycarbonyl |
| Phe | phenylalanine |
| 2-PhⁱPr | 2-phenylisopropyl |
| pHP | p-hydroxyphenacyl |
| Pmbf | 2,2,4,6,7-pentamethyl-5-dihydrobenzofuranyl- |
| | methyl |
| Pmc | 2,2,5,7,8-pentamethylchroman-6-sulfonyl |
| Pms | 2-[phenyl(methyl)sulfonio] ethyloxycarbonyl |
| | tetrafluoroborate |
| pNB | p-nitrobenzyl |
| pNBS | p-nitrobenzenesulfonyl |
| pNZ | p-nitrobenzyloxycarbonyl |
| Poc | propargyloxycarbonyl |
| Pro | proline |
| PTSA | p-toluenesulfonic acid |
| Pydec | 2-pyridyldithioethyloxy carbonyl |
| Ser | serine |
| Sps | 2-(4-sulfophenylsulfonyl) ethoxycarbonyl |
| S—Pyr | 2-pyridinesulfenyl |
| S^{t}Bu | tert-butylmercapto |
| Sub | 5-dibenzosuberyl |
| Suben | ω-5-dibenzosuberenyl |
| T₃P | propanephosphonic anhydride |
| TBDMS | tert-butyldimethylsilyl |
| TBDPS | tert-butyldiphenylsilyl |
| ^{t}Bu | tert-butyl |
| TBAF | tetrabutylammonium fluoride |
| TBE | 2,2,2-tribromoethyl |
| TBP | tri-n-butylphosphine |
| TCE | 2,2,2-trichloroethyl |
| TEA | triethylamine |
| Teoc | Trimethylsilylethoxy carbonyl |
| TFA | trifluoroacetic acid |
| TFMSA | trifluoromethanesulfonic acid |
| THF | tetrahydrofuran |
| Thr | threonine |
| TMA | trimethylamine |
| TMAC | trimethylacetyl chloride |
| Tmob | 2,4,6-trimethoxybenzyl |
| TMSE | trimethylsilylethyl |
| Tmsi | 2-(trimethylsilyl)isopropyl |
| Ts | Tosyl or p-tosyl (a.k.a. p-toluenesulfonyl) |
| Troc | 2,2,2-trichloroethyloxycarbonyl |
| Trp | tryptophan |
| Trt | trityl |
| Tyr | tyrosine |
| Val | valine |
| Xan | 9-xanthenyl |
| Z or cbz or Cbz | benzyloxycarbonyl |

Definitions of specific functional groups and chemical terms are described in more detail below. The chemical elements are identified in accordance with the Periodic Table of the Elements, GAS version, Handbook of Chemistry and Physics, 7Sh Ed., inside cover. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

The abbreviations used herein have their conventional meaning within the chemical and biological arts. The chemical structures and formulae set forth herein are intended to comply to the standard rules of chemical valency known in the chemical arts. When a range of values is listed, it is intended to encompass each value and subrange within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₅, C₃-C₅, C₃-C₄, C₄-C₆, C₄-C₅, and C₅-C₆ alkyl. When a group or moiety is referred to as “substituted”, one or more of the hydrogen atoms of the group has been replaced with a substituent. Possible “substituents” include, for example one or more: (i) D, F, Cl, Br or I atoms; or (ii) methyl, ethyl, propyl, trichloromethyl, trifluoromethyl, carbonyl (i.e. C═O), nitrile (i.e. —CN), hydroxyl (i.e. —OH), alkoxy (i.e. —OR″), nitro (i.e. —NO₂) or amino groups, each independently chosen for each possible position for substitution of a hydrogen atom. Other substituents are contemplated, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. A group or moiety that is not substituted is unsubstituted.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. Certain compounds of the present invention may exist in various tautomeric forms. Certain compounds of the present invention may exist in various salt forms. In general, all physical forms are equivalent for the uses contemplated by the present invention and are intended to be within the scope of the present invention.

As used herein, “acyl” (a.k.a. “alkanoyl”) refers to an alkyl, aryl, arylalkyl, cycloalkyl or heteroalkyl group with a linked terminal carbonyl group of general formula:

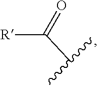

wherein R′ represents the alkyl, aryl, arylalkyl, arylheteroalkyl, cycloalkyl, heteroalkyl group or heteroaryheteroalkyl and [symbol] identifies the bond that forms the point of attachment of the group to another compound or moiety. Non-limiting examples of acyl groups include: formyl (C₁), acetyl (C₂), propionyl (C₃), 3-methoxypropanoyl (C₄ heteroalkyl), benzoyl (C₆ aryl), cyclohexanoyl, (C₇ cycloalkyl) and adamantoyl (C₁₁ biscyclic alkyl).

As used herein “acyloxy” refers to an acyl group linked to a terminal oxygen of general formula:

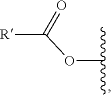

wherein R′ represents an alkyl, aryl, arylalkyl, cycloalkyl or heteroalkyl group and [symbol] identifies the bond that forms the point of attachment of the group to another compound or moiety.

As used herein “alkoxy” is one example of a heteroalkyl group and refers to an alkyl, cycloalkyl, heteroalkyl or cycloheteroalkyl group linked to a terminal oxygen of general formula:

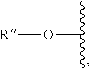

wherein R″ is the alkyl, cycloalkyl, heteroalkyl or cycloheteroalkyl group and [symbol] identifies the bond that forms the point of attachment of the group to another compound or moiety.

As used herein, “alkyl” refers to a radical of a straight-chain or branched saturated hydrocarbon group having from 1 to 30 carbon atoms (“C₁-C₂₀ alkyl”). In some embodiments, an alkyl group has 1 to 20 carbon atoms (“C₁-C₂₀ alkyl”). In some embodiments, an alkyl group has 1 to 15 carbon atoms (“C₁-C₁₅ alkyl”). In some embodiments, an alkyl group has 1 to 10 carbon atoms (“C₁-C₁₀ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 1 to 5 carbon atoms (“C₁-C₈ alkyl”). In some embodiments, an alkyl group has 1 to 4 carbon atoms (“C₁-C₄ alkyl”). In some embodiments, an alkyl group has 1 to 3 carbon atoms (“C₁-C₃ alkyl”). In some embodiments, an alkyl group has 1 to 2 carbon atoms (“C₁-C₂ alkyl”). In some embodiments, an alkyl group has 1 carbon atom (“C₁ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of higher order alkyl groups include n-heptyl (C₇), n-octyl (C₈), nonyl (C₉), decyl (C₁₀), undecyl (C₁₁) and dodecyl (C₁₂) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 4 substituents, 1 to 3 substituents, 1 to 2 substituents or just 1 substituent.

As used herein, “alkenyl” refers to a radical of a straight-chain or branched hydrocarbon group having from 2 to 12 carbon atoms, one or more carbon-carbon double bonds, and no triple bonds (“C₂-C₁₂ alkenyl”). In some embodiments, an alkenyl group has 1-10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 to 5 carbon atoms (“C₂-C₅ alkenyl”). In some embodiments, an alkenyl group has 2 to 4 carbon atoms (“C₂-C₄ alkenyl”). In some embodiments, an alkenyl group has 2 to 3 carbon atoms (“C₂-C₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂-C₄ alkenyl groups as well as pentenyl (C₅), pentadienyl (C₅), hexenyl (C₆), and the like. Additional examples of alkenyl include heptenyl (C₁), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents; e.g., for instance from 1 to 5 substituents, 1 to 4 substituents, 1 to 3 substituents, 1 to 2 substituents or just 1 substituent. For example, in certain embodiments, the alkenyl group can be an unsubstituted C₂-C₁₀ alkenyl and in certain embodiments, the alkenyl group can be a substituted C₂-C₆ alkenyl.

As used herein, the term “alkynyl” refers to a radical of a straight-chain or branched hydrocarbon group having from 2 to 12 carbon atoms, one or more carbon-carbon triple bonds (“C₂-C₁₂ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 to 5 carbon atoms (“C₂-C₅ alkynyl”). In some embodiments, an alkynyl group has 2 to 4 carbon atoms (“C₂-C₄ alkynyl”). In some embodiments, an alkynyl group has 2 to 3 carbon atoms (“C₂-C₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents; e.g., for instance from 1 to 5 substituents, 1 to 4 substituents, 1 to 3 substituents, 1 to 2 substituents or just 1 substituent. For example, in certain embodiments, the alkynyl group can be an unsubstituted C₂-10 alkynyl and in certain embodiments, the alkynyl group can be a substituted C₂-C₆ alkynyl.

As used herein, “aryl” (sometimes abbreviated as “Ar”) refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents; e.g., for instance from 1 to 5 substituents, 1 to 4 substituents, 1 to 3 substituents, 1 to 2 substituents or just 1 substituent. The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. For example, in certain embodiments, the aryl group can be an unsubstituted C₅-C₁₂ aryl and in certain embodiments, the aryl group can be a substituted C₅-C₁₀ aryl.

As used herein, the term “arylalkyl” refers to a radical of an aryl or heteroaryl group that is attached to a (C₁-C₁₂)alkyl group via an alkylene linker. As used herein, the term “arylalkyl” refers to a group that may be substituted or unsubstituted. The term “arylalkyl” is also intended to refer to those compounds wherein one or more methylene groups in the alkyl chain of the arylalkyl group can be replaced by a heteroatom such as O, N, P, Si, and S, and wherein the nitrogen, phosphorus and sulfur atoms may optionally be oxidized and the nitrogen heteroatom may optionally be quaternized with appended alkyl and/or aryl groups. Arylalkyl groups include for example, benzyl.

As used herein, the term “arylheteroalkyl” refers to a radical of aryl group linked to a non-cyclic stable straight or branched chain, or combinations thereof, including at least one carbon atom and at least one heteroatom selected from the group consisting of O, N, P, Si, and S, and wherein the nitrogen, phosphorus and sulfur atoms may optionally be oxidized, and the nitrogen heteroatom may optionally be quaternized with appended alkyl and/or aryl groups.

As used herein, “cycloalkyl” refers to a radical of a non-aromatic cyclic hydrocarbon group having from 3 to 12 ring carbon atoms (“C₃-C₁₂ cycloalkyl”). In some embodiments, a cycloalkyl group has 3 to 10 ring carbon atoms (“C₃-C₁₀ cycloalkyl”). In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 5 to 7 ring carbon atoms (“C₅-C₇ cycloalkyl”). A cycloalkyl group maybe described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₇ cycloalkyl groups include, without limitation, the aforementioned C₃-C₈ cycloalkyl groups as well as cycloheptyl (C₆), cycloheptenyl (C₇), cycloheptadienyl (C₇), and cycloheptatrienyl (C₇), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₇ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“biscyclic cycloalkyl”) and can be saturated or can be partially unsaturated. Non-limiting examples of biscyclic cycloalkyl groups include 1-ethylbicyclo[1.1.1]pentane, 1-ethylbicyclo[2.2.2]octane and (3r,5r,7r)-1-ethyladamantane. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents.

As used herein, “cycloheteroalkyl” refers to a radical of a cycloalkyl group comprising at least one heteroatom selected from the group consisting of O, N, P, Si, and S, and wherein the nitrogen, phosphorus and sulfur atoms may optionally be oxidized, and the nitrogen heteroatom may optionally be quaternized with appended alkyl and/or aryl groups. The heteroatom(s) O, N, P, S, and Si may be placed at any position of the cycloheteroalkyl group.

As used herein, the term “heteroalkyl” refers to a radical of a non-cyclic stable straight or branched chain, or combinations thereof, including at least one carbon atom and at least one heteroatom selected from the group consisting of O, N, P, Si, and S, and wherein the nitrogen, phosphorus and sulfur atoms may optionally be oxidized, and the nitrogen heteroatom may optionally be quaternized with appended alkyl and/or aryl groups. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH₂—CH₂—P(O)₂—CH₃, —CH═CH—O—CH₃, —Si(CH₃)₃, —CH₂—CH═N—OCH₃, —CH═CH—N(CH₃)—CH₃, —O—CH₃, and —O—CH₂—CH₃. Up to two heteroatoms may be consecutive, such as, for example, —CH₂—NH—OCH₃, —CH₂CH₂—S—S—CH₂CH₃ and —CH₂—O—Si(CH₃)₃. Each instance of heteroalkyl group may be independently optionally substituted, i.e., unsubstituted (an “unsubstituted heteroalkyl”) or substituted (a “substituted heteroalkyl”) with one or more substituents; e.g., for instance from 1 to 5 substituents, 1 to 4 substituents, 1 to 3 substituents, 1 to 2 substituents or just 1 substituent.

As used herein, the term “heteroaryl” refers to a radical of an aromatic heterocycle that comprises 1, 2, 3 or 4 heteroatoms selected, independently of the others, from nitrogen, sulfur and oxygen. As used herein, the term “heteroaryl” refers to a group that may be substituted or unsubstituted. A heteroaryl may be fused to one or two rings, such as a cycloalkyl, an aryl, or a second heteroaryl ring. The point of attachment of a heteroaryl to a molecule may be on the heteroaryl, cycloalkyl, heterocycloalkyl or aryl ring, and the heteroaryl group may be attached through carbon or a heteroatom. Examples of heteroaryl groups include imidazolyl, furyl, pyrrolyl, thienyl, thiazolyl, isoxazolyl, isothiazolyl, thiadiazolyl, oxadiazolyl, pyridinyl, pyrimidyl, pyrazinyl, pyridazinyl, quinolyl, isoquinolinyl, indazolyl, benzoxazolyl, benzisooxazolyl, benzofuryl, benzothiazolyl, indolizinyl, imidazopyridinyl, pyrazolyl, triazolyl, oxazolyl, tetrazolyl, benzimidazolyl, benzoisothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidyl, pyrazolo[3,4]pyrimidyl or benzo(b)thienyl, each of which can be optionally substituted. The aromatic heterocycle may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like.

As used herein, the term “heteroarylheteroalkyl” refers to a radical of a heteroaryl group linked to a heteroalkyl group wherein the heteroalkyl group is the point of attachment to the atom or moiety of interest.

As used herein, the term “heterocyclic ring” or “heterocycle” refers to a ring of atoms of at least two different elements, one of which is carbon. Additional reference is made to: Oxford Dictionary of Biochemistry and Molecular Biology, Oxford University Press, Oxford, 1997 as evidence that the term “heterocyclic ring” is a term well-established in field of organic chemistry.

As used herein, the term “hydrate” refers to a compound which is associated with water. Typically, the number of the water molecules contained in a hydrate of a compound is in a definite ratio to the number of the compound molecules in the hydrate.

As used herein, the term “protecting group” refers to a chemical group that is reacted with, and bound to (at least for some period of time), a functional group in a molecule to prevent said functional group from participating in reactions of the molecule but which chemical group can subsequently be removed to thereby regenerate said functional group. Additional reference is made to: Oxford Dictionary of Biochemistry and Molecular Biology, Oxford University Press, Oxford, 1997 as evidence that protecting group is a term well-established in field of organic chemistry. Further reference is made to Greene's Protective Groups in Organic Synthesis, Fourth Edition, 2007, John Wiley & Sons, Inc. which is known as a primary reference for researching the suitability of various protecting groups in organic synthesis reactions. Further reference is also made to: Isidro-Llobet, A., Alvarez, M., Albericio, F., “Amino Acid-Protecting Groups”; Chem. Rev., 109: 2455-2504 (2009) as a comprehensive review of protecting groups commonly used in peptide synthesis. As used herein, the term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like

As used herein, the term “tautomer” as used herein refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Tautomeric forms may be relevant to the attainment of the optimal chemical reactivity and biological activity of a compound of interest.

### Chiral/Stereochemistry Considerations

Compounds described herein can comprise one or more asymmetric centers, and thus can exist in various isomeric forms, e.g., enantiomers and/or diastereomers. Chiral centers in illustrated structures may be identified herein by use of an asterisk (*). For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

### R (D for an Amino Acid) or S (L for an Amino Acid)

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. With respect to amino acids (which are more commonly described in terms of “D” and “L” enantiomer, it is to be understood that for a “D”-amino acid the configuration is “R” and for an “L”-amino acid, the configuration is “S”. In some embodiments, ‘substantially free’, refers to: (i) an aliquot of an “R” form compound that contains less than 2% “S” form; or (ii) an aliquot of an “S” form compound that contains less than 2% “R” form. The term “enantiomerically pure” or “pure enantiomer” denotes that the compound comprises more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure “R” form compound can comprise, for example, about 90% excipient and about 10% enantiomerically pure “R” form compound. In certain embodiments, the enantiomerically pure “R” form compound in such compositions can, for example, comprise, at least about 95% by weight “R” form compound and at most about 5% by weight “S” form compound, by total weight of the compound. For example, a pharmaceutical composition comprising enantiomerically pure “S” form compound can comprise, for example, about 90% excipient and about 10% enantiomerically pure “S” form compound. In certain embodiments, the enantiomerically pure “S” form compound in such compositions can, for example, comprise, at least about 95% by weight “S” form compound and at most about 5% by weight “R” form compound, by total weight of the compound. In certain embodiments, the active ingredient can be formulated with little or no excipient or carrier.

The nomenclature used to define the peptide compounds described herein is that typically used in the art wherein the amino group at the N-terminus appears to the left and the carboxyl group at the C-terminus appears to the right.

As used herein, the term “amino acid” includes both a naturally occurring amino acid and a non-natural amino acid. The term “amino acid,” unless otherwise indicated, includes both isolated amino acid molecules (i.e., molecules that include both, an amino-attached hydrogen and a carbonyl carbon-attached hydroxyl) and residues of amino acids (i.e., molecules in which either one or both an amino-attached hydrogen or a carbonyl carbon-attached hydroxyl are removed). The amino group can be alpha-amino group, beta-amino group, etc. For example, the term “amino acid alanine” can refer either to an isolated alanine H-Ala-OH or to any one of the alanine residues H-Ala-, -Ala-OH, or -Ala-. Unless otherwise indicated, all amino acids found in the compounds described herein can be either in D or L configuration. An amino acid that is in D configuration may be written such that “D” precedes the amino acid abbreviation. For example, “D-Arg” represents arginine in the D configuration. The term “amino acid” includes salts thereof, including pharmaceutically acceptable salts. Any amino acid can be protected or unprotected. Protecting groups can be attached to an amino group (for example alpha-amino group), the backbone carboxyl group, or any functionality of the side chain. As an example, phenylalanine protected by a benzyloxycarbonyl group (Z) on the alpha-amino group would be represented as Z-Phe-OH.

With the exception of the N-terminal amino acid, all abbreviations of amino acids (for example, Phe) in this disclosure stand for the structure of —NH—C(R)(W)—CO—, wherein R and R′ each is, independently, hydrogen or the side chain of an amino acid (e.g., R=benzyl and R′═H for Phe). Accordingly, phenylalanine is H-Phe-OH. The designation “OH” for these amino acids, or for peptides (e.g., Lys-Val-Leu-OH) indicates that the C-terminus is the free acid. The designation “NH₂” in, for example, Phe-D-Arg-Phe-Lys-NH₂ indicates that the C-terminus of the protected peptide fragment is amidated. Further, certain R and R′, separately, or in combination as a ring structure, can include functional groups that require protection during the liquid phase synthesis.

Where the amino acid has isomeric forms, it is the L form of the amino acid that is represented unless otherwise explicitly indicated as D form, for example, D-Arg. Notably, many amino acid residues are commercially available in both D- and L-form. For example, D-Arg is a commercially available D-amino acid.

A capital letter “D” used in conjunction with an abbreviation for an amino acid residue refers to the D-form of the amino acid residue.

As used herein, the term “peptide” refers to two or more amino acids covalently linked by at least one amide bond (i.e., a bond between an amino group of one amino acid and a carboxyl group of another amino acid selected from the amino acids of the peptide fragment). The term “peptide” includes salts thereof, including pharmaceutically acceptable salts.

The term “DMT”, 2,6-DMT or 2,6-Dmt refers to 2,6-di(methyl)tyrosine (e.g., 2,6-dimethyl-L-tyrosine; CAS 123715-02-6).

The term “Nva” refers to norvaline, a/k/a 2-aminopentanoic acid (CAS 6600-40-4). Norvaline has two enantiomeric forms, which may be termed D- and L-norvaline. Additionally, and for example, the name “δ-(substituent)-Nva” or “5-(substituent)-Nva” refers to a norvaline in which the designated substituent replaces a hydrogen atom on the δ- or 5-carbon of norvaline. Other substitution patterns are possible, which are named in a similar fashion.

The term “Agb” refers to 2-amino-4-guanidino-butyric acid (e.g., 2-amino-4-guanidino-D-butyric acid), a homologue of Arg.

The articles “a” and “an” are used herein to refer to one or to more than one (i.e., to at least one) of the grammatical object of the article. By way of example, “an element” means one element or more than one element.

The invention also provides salts of the compounds of the invention.

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound or three hydrochloric acid molecules per molecule of compound. In some embodiments, the compound may comprise, one hydrochloric acid molecule per molecule of compound, two hydrochloric acid molecules per molecule of compound or three hydrochloric acid molecules per molecule of compound. In some embodiments, the compound may comprise, one acetic acid molecule per molecule of compound, two acetic acid molecules per molecule of compound or three acetic acid molecules per molecule of compound. In some embodiments, the compound may comprise, one trifluoroacetic acid molecule per molecule of compound, two trifluoroacetic acid molecules per molecule of compound or three trifluoroacetic acid molecules per molecule of compound. As another example, the salt may comprise less than one inorganic or organic acid molecule per molecule of base, such as two molecules of compound per molecule of tartaric acid. “Pharmaceutically acceptable salt” also refers to salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tosylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention. In some embodiments, a pharmaceutically acceptable salt is a benzenesulfonic acid salt, a p-tosylsulfonic acid salt, or a methanesulfonic acid salt.

As used herein, the term “prodrug” as used herein encompasses compounds that, under physiological conditions, are converted into therapeutically active agents. A common method for making a prodrug is to include selected moieties that are cleavable under physiological conditions to reveal the desired active molecule in vivo. In other embodiments, the prodrug is converted by an enzymatic activity of the host animal. This approach may improve the physicochemical property of the active molecule, including its PK/ADME profile. The approach could also alter the side-effect profile of the active molecule, while maintaining desired efficacy for the treatment.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

As used herein, “inhibit” or “inhibiting” means reduce by an objectively measureable amount or degree compared to control. In one embodiment, inhibit or inhibiting means reduce by at least a statistically significant amount compared to control. In one embodiment, inhibit or inhibiting means reduce by at least 5 percent compared to control. In various individual embodiments, inhibit or inhibiting means reduce by at least 10, 15, 20, 25, 30, 33, 40, 50, 60, 67, 70, 75, 80, 90, 95, or 99 percent compared to control.

As used herein, the terms “treating” and “treat” refer to performing an intervention that results in (a) preventing a condition or disease from occurring in a subject that may be at risk of developing or predisposed to having the condition or disease but has not yet been diagnosed as having it; (b) inhibiting a condition or disease, e.g., slowing or arresting its development or progression; or (c) relieving or ameliorating a condition or disease, e.g., causing regression of the condition or disease. In one embodiment the terms “treating” and “treat” refer to performing an intervention that results in (a) inhibiting a condition or disease, e.g., slowing or arresting its development; or (b) relieving or ameliorating a condition or disease, e.g., causing regression of the condition or disease.

As used herein, a “subject” refers to a living animal. In various embodiments, a subject is a mammal. In various embodiments, a subject is a non-human mammal, including, without limitation, a mouse, rat, hamster, guinea pig, rabbit, sheep, goat, cat, dog, pig, horse, cow, or non-human primate. In certain embodiments, the subject is a human.

As used herein, “administering” has its usual meaning and encompasses administering by any suitable route of administration, including, without limitation, intravenous, intramuscular, intraperitoneal, subcutaneous, direct injection, mucosal, inhalation, oral, and topical.

As used herein, the phrase “effective amount” refers to any amount that is sufficient to achieve a desired biological effect. A “therapeutically effective amount” is an amount that is sufficient to achieve a desired therapeutic effect, e.g., to treat ischemia-reperfusion injury.

Compounds of the invention and the salts thereof can be combined with other therapeutic agents. The compounds of the invention and other therapeutic agent may be administered simultaneously or sequentially. When the other therapeutic agents are administered simultaneously, they can be administered in the same or separate formulations, but they are administered substantially at the same time. The other therapeutic agents are administered sequentially with one another and with compounds of the invention, when the administration of the other therapeutic agents and the compound of the invention is temporally separated. The separation in time between the administration of these compounds may be a matter of minutes or it may be longer.

### Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13 (suppl. 5):143-146 (1989) (endothelin-1); Hubbard et al., Annal Int Med 3:206-212 (1989) (al-antitrypsin); Smith et al., 1989, J Chn Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

Methods of Use

The present invention provides prodrug of a non-natural peptide compound useful for treating or preventing ischemia-reperfusion injury or myocardial infarction, or injury associated with myocardial infarction.

Accordingly, in certain embodiments, the invention is directed to a method of treating or preventing ischemia-reperfusion injury, comprising administering to a subject in need thereof a prodrug of a therapeutically effective amount of a non-natural peptide compound, or a pharmaceutically acceptable salt thereof. In certain such embodiments, the ischemia-reperfusion injury is cardiac ischemia-reperfusion injury. In some embodiments, the compound is administered orally, topically, systemically, intravenously, subcutaneously, intraperitoneally, or intramuscularly.

In other embodiments, the present invention provides a method for treating or preventing a myocardial infarction, comprising administering to a subject in need thereof a therapeutically effective amount of compound of formula (I), or a pharmaceutically acceptable salt thereof. Such methods may prevent injury to the heart upon reperfusion by preventing the initiation or progression of the infarction. In some embodiments, the compound is administered orally, topically, systemically, intravenously, subcutaneously, intraperitoneally, or intramuscularly

Ischemia is reduction or decrease in blood supply to a tissue or an organ and has many different causes. Ischemia may be local, e.g., caused by thrombus or embolus, or more global, e.g., due to low perfusion pressure. An ischemic event can lead to hypoxia (reduced oxygen) and/or anoxia (absence of oxygen).

Ischemia in a tissue or organ of a mammal is a multifaceted pathological condition that is caused by oxygen deprivation (hypoxia) and/or glucose (e.g., substrate) deprivation. Oxygen and/or glucose deprivation in cells of a tissue or organ leads to a reduction or total loss of energy generating capacity and consequent loss of function of active ion transport across the cell membranes. Oxygen and/or glucose deprivation also leads to pathological changes in other cell membranes, including permeability transition in the mitochondrial membranes. In addition, other molecules, such as apoptotic proteins normally compartmentalized within the mitochondria, may leak out into the cytoplasm and cause apoptotic cell death. Profound ischemia can lead to necrotic cell death.

Ischemia or hypoxia in a particular tissue or organ may be caused by a loss or severe reduction in blood supply to the tissue or organ. The loss or severe reduction in blood supply may, for example, be due to thromboembolic stroke, coronary atherosclerosis, or peripheral vascular disease. The tissue affected by ischemia or hypoxia is typically muscle, such as cardiac, skeletal, or smooth muscle.

The organ affected by ischemia or hypoxia may be any organ that is subject to ischemia or hypoxia. By way of example, but not by way of limitation, cardiac muscle ischemia or hypoxia is commonly caused by atherosclerotic or thrombotic blockages, which lead to the reduction or loss of oxygen delivery to the cardiac tissues by the cardiac arterial and capillary blood supply. Such cardiac ischemia or hypoxia may cause pain and necrosis of the affected cardiac muscle, and ultimately may lead to cardiac failure.

Reperfusion is the restoration of blood flow to any organ or tissue in which the flow of blood is decreased or blocked. For example, blood flow can be restored to any organ or tissue affected by ischemia. The restoration of blood flow (reperfusion) can occur by any method known to those in the art. For instance, reperfusion of ischemic cardiac tissues may arise from angioplasty, coronary artery bypass graft, or the use of thrombolytic drugs.

Ischemia-reperfusion injury is the cellular or tissue damage caused when blood supply returns to the affected area after a period of ischemia. The lack of oxygen and nutrients during ischemia creates a condition in which the restoration of circulation results damage to the tissues. By way of example, but not by way of limitation, forms of myocardial reperfusion injury including reperfusion-induced arrhythmias, myocardial stunning, microvascular obstruction manifesting in sluggish coronary blood flow, and lethal myocardial reperfusion injury (i.e., reperfusion-induced death of cardiomyocytes that were viable at the end of the index ischemic event). Studies have suggested that lethal myocardial reperfusion injury accounts for about 50% of the final myocardial infarct size.

In certain embodiments, the peptide is administered orally, intravenously, or parenterally.

In certain embodiments, the subject is a human.

A non-natural peptide compound of the invention, or a pharmaceutically acceptable salt thereof, such as acetate, tartrate, or trifluoroacetate salt, may be administered to a subject suspected of, or already suffering from ischemic injury in an amount sufficient to cure, or at least partially arrest, the symptoms of the disease, including its complications and intermediate pathological phenotypes in development of the disease. Subjects suffering from ischemic injury can be identified by any or a combination of diagnostic or prognostic assays known in the art. By way of example, but not by way of limitation, in some embodiments, the ischemic injury is related to cardiac ischemia, brain ischemia, renal ischemia, cerebral ischemia, intestinal ischemia, hepatic ischemia, or myocardial infarction.

By way of example, but not by way of limitation, typical symptoms of cardiac ischemia include, but are not limited to, angina (e.g., chest pain and pressure), shortness of breath, palpitations, weakness, dizziness, nausea, sweating, rapid heartbeat, and fatigue.

In some embodiments, treatment of subjects diagnosed with cardiac ischemia with at least one peptide disclosed herein ameliorates or eliminates of one or more of the following symptoms of cardiac ischemia: angina (e.g., chest pain and pressure), shortness of breath, palpitations, weakness, dizziness, nausea, sweating, rapid heartbeat, and fatigue.

By way of example, but not by way of limitation, typical symptoms of renal ischemia include, but are not limited to, uremia (i.e., high blood levels of protein by-products, such as, e.g., urea), acute episodes of dyspnea (labored or difficult breathing) caused by sudden accumulation of fluid in the lungs, hypertension, pain felt near the kidneys, weakness, hypertension, nausea, a history of leg pain, a stride that reflects compromised circulation to the legs, and bruits (sound or murmurs heard with a stethoscope) caused by turbulent blood flow within the arteries may be detected in the neck (e.g., carotid artery bruit), abdomen (which may reflect narrowing of the renal artery), and groin (femoral artery bruit).

In some embodiments, treatment of subjects diagnosed with renal ischemia with at least one peptide disclosed herein ameliorates or eliminates of one or more of the following symptoms of renal ischemia: uremia (i.e., high blood levels of protein by-products, such as, e.g., urea), acute episodes of dyspnea (labored or difficult breathing) caused by sudden accumulation of fluid in the lungs, hypertension, pain felt near the kidneys, weakness, hypertension, nausea, a history of leg pain, a stride that reflects compromised circulation to the legs, and bruits (sound or murmurs heard with a stethoscope) caused by turbulent blood flow within the arteries may be detected in the neck (e.g., carotid artery bruit), abdomen (which may reflect narrowing of the renal artery), and groin (femoral artery bruit).

By way of example, but not by way of limitation, typical symptoms of cerebral (or brain) ischemia include, but are not limited to, blindness in one eye, weakness in one arm or leg, weakness in one entire side of the body, dizziness, vertigo, double vision, weakness on both sides of the body, difficulty speaking, slurred speech, and the loss of coordination.

In some embodiments, treatment of subjects diagnosed with cerebral (or brain) ischemia with at least one peptide disclosed herein ameliorates or eliminates of one or more of the following symptoms of cerebral (or brain) ischemia: blindness in one eye, weakness in one arm or leg, weakness in one entire side of the body, dizziness, vertigo, double vision, weakness on both sides of the body, difficulty speaking, slurred speech, and the loss of coordination.

In another aspect, the present invention relates to methods of treating ischemia reperfusion injury and/or side effects associated with existing therapeutics against ischemia reperfusion injury. In therapeutic applications, a composition or medicament comprising at least one compound of the invention, or a pharmaceutically acceptable salt thereof, such as acetate, tartrate or trifluoroacetate, is administered to a subject suspected of, or already suffering from ischemic reperfusion injury in an amount sufficient to cure, or at least partially arrest, the symptoms of the disease, including its complications and intermediate pathological phenotypes in development of the disease. Subjects suffering from ischemic-reperfusion injury can be identified by any or a combination of diagnostic or prognostic assays known in the art. In some embodiments, the ischemia-reperfusion injury is related to cardiac ischemia, brain ischemia, renal ischemia, cerebral ischemia, intestinal ischemia, and hepatic ischemia. In some embodiments, the compounds disclosed herein are useful in the treatment of cardiac ischemia-reperfusion injury.

In some embodiments, the cyclic peptide compounds disclosed herein are useful in treating myocardial infarction in a subject to prevent injury to the heart upon reperfusion. In some embodiments, the invention relates to methods of coronary revascularization, comprising administering to a mammalian subject a therapeutically effective amount of a compound of the invention, or a pharmaceutically acceptable salt thereof, and performing a coronary artery bypass graft (CABG) procedure on the subject.

In some embodiments, treatment of myocardial infarction with the compounds disclosed herein reduces infarct size, increases LVDP, and increases maximal rates of contraction and relaxation (±dP/dt).

In still yet further embodiments, the invention provides a method for treating or preventing hind limb or critical limb ischemia in a subject in need thereof, comprising administering to the subject a therapeutically effective amount of a compound of the invention.

In any of the foregoing embodiments, the compound of the invention may be administered orally, topically, systemically, intravenously, subcutaneously, intraperitoneally, or intramuscularly.

### Prophylactic Methods

In some embodiments, the present invention provides methods for preventing or delaying the onset of ischemic injury or symptoms of ischemic injury in a subject at risk of having ischemia injury. In some embodiments, the present technology provides methods for preventing or reducing the symptoms of ischemic injury in a subject at risk of having ischemia injury.

In some embodiments, the present invention provides methods for preventing or delaying the onset of ischemia-reperfusion injury or symptoms of ischemia-reperfusion injury in a subject at risk of having ischemia-reperfusion injury. In some embodiments, the present invention provides methods for preventing or reducing the symptoms of ischemia reperfusion injury in a subject at risk of having ischemia-reperfusion injury.

In some embodiments, the ischemic injury, the ischemia-reperfusion injury, or symptoms of ischemic or ischemia-reperfusion injury is related to cardiac ischemia, brain ischemia, renal ischemia, cerebral ischemia, intestinal ischemia, and hepatic ischemia. In some embodiments, the ischemic injury is myocardial infarction.

In some embodiments, the cyclic peptide compounds disclosed herein are useful in the treatment or prevention of cardiac ischemia-reperfusion injury. In some embodiments, the compounds disclosed herein are useful in the prevention of cardiac ischemia-reperfusion injury.

Subjects at risk for ischemic injury or ischemia-reperfusion injury can be identified by, e.g., any or a combination of diagnostic or prognostic assays known in the art. In prophylactic applications, a pharmaceutical composition or medicament of a compound of the invention, or a pharmaceutically acceptable salt thereof, such as acetate, tartrate, or trifluoroacetate salt, is administered to a subject susceptible to, or otherwise at risk of for ischemic injury or ischemia reperfusion injury in an amount sufficient to eliminate, reduce the risk, or delay the onset of the disease, including biochemical, histologic and/or behavioral symptoms of the disease, its complications and intermediate pathological phenotypes presenting during development of the disease or reduce the symptoms and/or complications and intermediate pathological phenotypes presenting during development of the disease. Administration of a prophylactic peptide can occur prior to the manifestation of symptoms characteristic of the disease or disorder, such that the disease or disorder is prevented, delayed in its progression, or the severity of the symptoms or side effects of the disease or disorder are reduced.

By way of example, in some embodiments, subjects may be at risk for cardiac ischemia if they have coronary artery disease (atherosclerosis), blood clots, or coronary artery spasm.

By way of example, but not by way of limitation, in some embodiments, subjects may be at risk for renal ischemia if they have kidney injury (e.g., acute kidney injury) and/or injuries or complications from surgeries in which the kidneys are deprived of normal blood flow for extended periods of time (e.g., heart-bypass surgery).

By way of example, but not by way of limitation, in some embodiments, subjects may be at risk for cerebral ischemia if they have sickle cell anemia, compressed blood vessels, ventricular tachycardia, plaque buildup in the arteries, blood clots, extremely low blood pressure as a result of heart attack, had a stroke, or congenital heart defects.

For therapeutic and/or prophylactic applications, a composition comprising at least one cyclic peptide compound described herein, or a pharmaceutically acceptable salt thereof, such as acetate, tartrate, or trifluoroacetate salt, is administered to a subject in need thereof. In some embodiments, the peptide composition is administered one, two, three, four, or five times per day. In some embodiments, the peptide composition is administered more than five times per day. Additionally or alternatively, in some embodiments, the peptide composition is administered every day, every other day, every third day, every fourth day, every fifth day, or every sixth day. In some embodiments, the peptide composition is administered weekly, bi-weekly, tri-weekly, or monthly. In some embodiments, the peptide composition is administered for a period of one, two, three, four, or five weeks. In some embodiments, the peptide is administered for six weeks or more. In some embodiments, the peptide is administered for twelve weeks or more. In some embodiments, the peptide is administered for a period of less than one year. In some embodiments, the peptide is administered for a period of more than one year. In some embodiments, treatment with at least one peptide disclosed herein will prevent or delay the onset of one or more of the following symptoms of cardiac ischemia: angina (e.g., chest pain and pressure), shortness of breath, palpitations, weakness, dizziness, nausea, sweating, rapid heartbeat, and fatigue.

In some embodiments, treatment with at least one peptide disclosed herein will prevent or delay the onset of one or more of the following symptoms of renal ischemia: uremia (i.e., high blood levels of protein by-products, such as, e.g., urea), acute episodes of dyspnea (labored or difficult breathing) caused by sudden accumulation of fluid in the lungs, hypertension, pain felt near the kidneys, weakness, hypertension, nausea, a history of leg pain, a stride that reflects compromised circulation to the legs, and bruits (sound or murmurs heard with a stethoscope) caused by turbulent blood flow within the arteries may be detected in the neck (e.g., carotid artery bruit), abdomen (which may reflect narrowing of the renal artery), and groin (femoral artery bruit).

In some embodiments, treatment with at least one peptide disclosed herein will prevent or delay the onset of one or more of the following symptoms of cerebral (or brain) ischemia: blindness in one eye, weakness in one arm or leg, weakness in one entire side of the body, dizziness, vertigo, double vision, weakness on both sides of the body, difficulty speaking, slurred speech, and the loss of coordination.

### EXAMPLES

### Example 1. Synthesis of Cyclic Tetrapeptide, (10S,13S,16R)-16-amino-N—((S)-1-amino oxo-3-phenylpropan-2-yl)-13-(4-hydroxy-2,6-dimethylbenzyl)-2-imino-4,12,15-trioxo-1,3,5,11,14-pentaazacyclononadecane-10-carboxamide (Compound A)

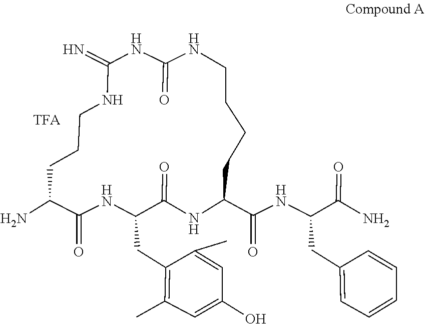 [SMILES: Cc1cc(O)cc(C)c1C[C@@H]1NC(=O)[C@H](N)CCCNC(=N)NC(=O)NCCCC[C@@H](C(=O)N[C@@H](Cc2ccccc2)C(N)=O)NC1=O]

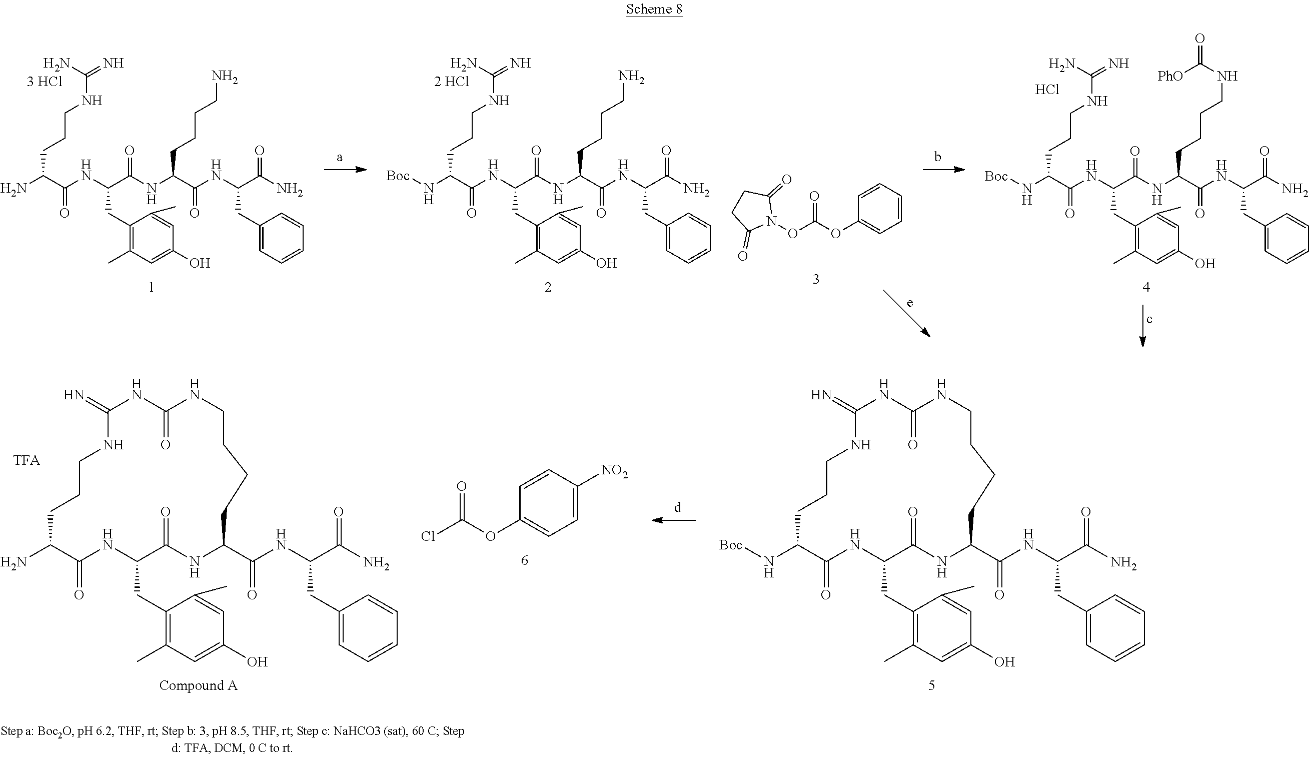

### 1) Step a. Synthesis of tert-butyl ((6R,9S,12S,15S)-1,16-diamino-12-(4-aminobutyl)-15-benzyl-9-(4-hydroxy-2,6-dimethylbenzyl)-1-imino-7,10,13,16-tetraoxo-2,8,11,14-tetraazahexadecan-6-yl)carbamate (D-(N2-Boc)-Arg-DMT-Lys-Phe-NH₂, 2)

To a solution of 1 ((S)-6-amino-N—((S)-1-amino-1-oxo-3-phenylpropan-2-yl)-2-((S)-2-((R)-2-amino-5-guanidinopentanamido)-3-(4-hydroxy-2,6-dimethylphenyl)propanamido)hexanamide, D-Arg-DMT-Lys-Phe-NH₂, 3.00 g, 4.0 mmol) in mixture of THF (50 mL), EtOH (10 mL) and Krebs-Ringer bicarbonate buffer (100 mL, pH 6.2, 1M) was added solution of Boc₂O (1.22 g, 5.6 mmol) in THF (20 mL). Reaction was stirred for 18 hours, then additionally was added solution of Boc₂O (1.22 g, 5.6 mmol) in THF (20 mL). After additional 10 hours to reaction mixture was added AcOH (to pH 6) and reaction mixture was evaporated. Crude product purified by reverse phase flash chromatography (eluent: H₂O (0.2% AcOH)/MeOH from 5% to 85% of methanol) to yield 2 (2.11 g, 66%) as white foam.

### 2) Step b: Synthesis of tert-butyl ((6R,9S,12S)-1-amino-12-O(S)-1-amino-1-oxo phenylpropan-2-yl)carbamoyl)-9-(4-hydroxy-2,6-dimethylbenzyl)-1-imino-7,10,18-trioxo-18-phenoxy-2,8,11,17-tetraazaoctadecan-6-yl)carbamate (D-(N2-Boc)-Arg-DMT-(N6-PhOCO)-Lys-Phe-NH₂, 4)

To solution of 2 (2.11 g, 2.6 mmol) in THF (260 mL) 2,5-dioxopyrrolidin-1-yl phenyl carbonate (3, 0.61 g, 2.6 mmol) solution in THF (20 mL) was added during period of 2 hours. Reaction was completed after 30 minutes (4 formed, monitoring with LC/MS). This reaction mixture was then used for the subsequent reaction without purification.

### 3) Step c: Synthesis of tert-butyl ((9R,12S,15S)-15-4(S)-1-amino-1-oxo-3-phenylpropan-2-yl)carbamoyl)-12-(4-hydroxy-2,6-dimethylbenzyl)-4-imino-2,10,13-trioxo-1,3,5,11,14-pentaazacyclononadecan-9-yl)carbamate (5)

A saturated sodium bicarbonate solution (13 mL) was added and reaction mixture from the previous step and stirred at 60° C. for 1 hour. Next, reaction mixture was cooled to 0° C., acidified with AcOH to pH 5 and then evaporated to dryness (re-evaporation with toluene).

### 4) Step d: Synthesis of (10S,13S,16R)-16-amino-N—((S)-1-amino-1-oxo-3-phenylpropan-2-yl)-13-(4-hydroxy-2,6-dimethylbenzyl)-2-imino-4,12,15-trioxo-1,3,5,11,14-pentaazacyclononadecane-10-carboxamide (Compound A)

Remaining solid from the previous step c was suspended in DCM (200 mL) under inert atmosphere and cooled to 0° C. Afterwards, to suspension was added TFA (20 mL) and reaction allowed warming to room temperature and stirring for 3 hours. When reaction was completed solvent was evaporated and crude product was purified by reverse phase flash chromatography (eluent: H₂O (0.2% AcOH)/MeOH from 5% to 85% of methanol) to yield 350 mg of crude Compound A (contains 5-8% epimer by NMR). Compound A was additionally purified by prep. HPLC to yield a pure product (125 mg, overall yield 6.5%, HPLC purity 97.0%) as white foam. ¹H NMR (400 MHz, Methanol-d₄) δ 7.36-7.16 (m, 5H), 6.40 (s, 2H), 4.49 (dd, J=8.6, 6.1 Hz, 1H), 4.41-4.23 (m, 2H), 4.05-3.72 (m, 1H), 3.24-3.02 (m, 4H), 2.94 (dt, J=13.8, 6.9 Hz, 2H), 2.14 (s, 6H), 2.04-1.25 (m, 11H). MS (M+H⁺): 666.54.

Alternatively, Compound A can be made via Step e and d (Schedule 1).

### 5) Step e: Synthesis of tert-butyl ((9R,12S,15S)-15-(((S)-1-amino-1-oxo-3-phenylpropan-2-yl)carbamoyl)-12-(4-hydroxy-2,6-dimethylbenzyl)-4-imino-2,10,13-trioxo-1,3,5,11,14-pentaazacyclononadecan-9-yl)carbamate (5)

To a solution of 2 (2.11 g, 2.6 mmol) in THF (150 mL) and Krebs-Ringer bicarbonate buffer (70 mL, pH 7.4, 1M) p-nitrophenyl chloroformate (6, 1.22 g, 6.06 mmol) in 200 mL of THF was added at 0° C. during 30 min. Then pH of the solution increased to 8.5 with saturated sodium bicarbonate solution and reaction stirred at 45° C. for 2 h. Then reaction mixture was cooled to 0° C., acidified with AcOH to pH 5 and evaporated to dryness (re-evaporation with toluene). The solid mixture was used for the next step reaction without further purification.

### 6) Step d: Synthesis of (10S,13S,16R)-16-amino-N—((S)-1-amino-1-oxo-3-phenylpropan-2-yl)-13-(4-hydroxy-2,6-dimethylbenzyl)-2-imino-4,12,15-trioxo-1,3,5,11,14-pentaazacyclononadecane-10-carboxamide (Compound A)

The remaining solid from step e was suspended in DCM (200 mL) under inert atmosphere and cooled to 0° C. Afterwards, to suspension was added TFA (20 mL) and reaction allowed warming to room temperature and stirring for 3 h. When reaction was completed solvent was evaporated and crude product was purified by reverse phase flash chromatography (eluent: H₂O (0.2% AcOH)/MeOH from 5% to 85% of methanol) to yield 610 mg of crude Compound A (contains 3-4% of epimer by NMR). Macrocycle Compound A was additionally purified by prep. HPLC to yield a pure product (170 mg, HPLC purity 97.6%) as white foam. ¹H NMR (400 MHz, Methanol-d₄) δ 7.36-7.16 (m, 5H), 6.40 (s, 2H), 4.49 (dd, J=8.6, 6.1 Hz, 1H), 4.41-4.23 (m, 2H), 4.05-3.72 (m, 1H), 3.24-3.02 (m, 4H), 2.94 (dt, J=13.8, 6.9 Hz, 2H), 2.14 (s, 6H), 2.04-1.25 (m, 11H). MS (M+H⁺): 666.54.

### Example 2. Synthesis of ethyl ((S)-6-(((S)-1-amino-1-oxo-3-phenylpropan-2-yl)amino)-5-((S)-2-((R)-2-amino-5-guanidinopentanamido)-3-(4-hydroxy-2,6-dimethylphenyl)propanamido)-6-oxohexyl)carbamate (D-Arg-DMT-(N6-Ethoxycarbonyl)-Lys-Phe-NH₂) (Compound B)

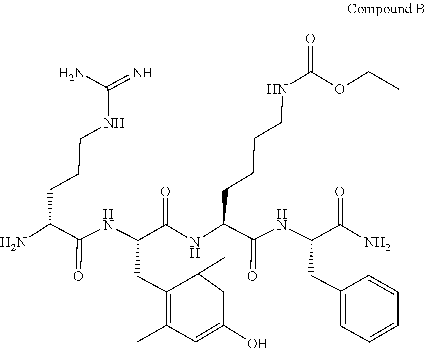 [SMILES: CCOC(=O)NCCCC[C@H](NC(=O)[C@H](CC1=C(C)C=C(O)CC1C)NC(=O)[C@H](N)CCCNC(=N)N)C(=O)N[C@@H](Cc1ccccc1)C(N)=O]

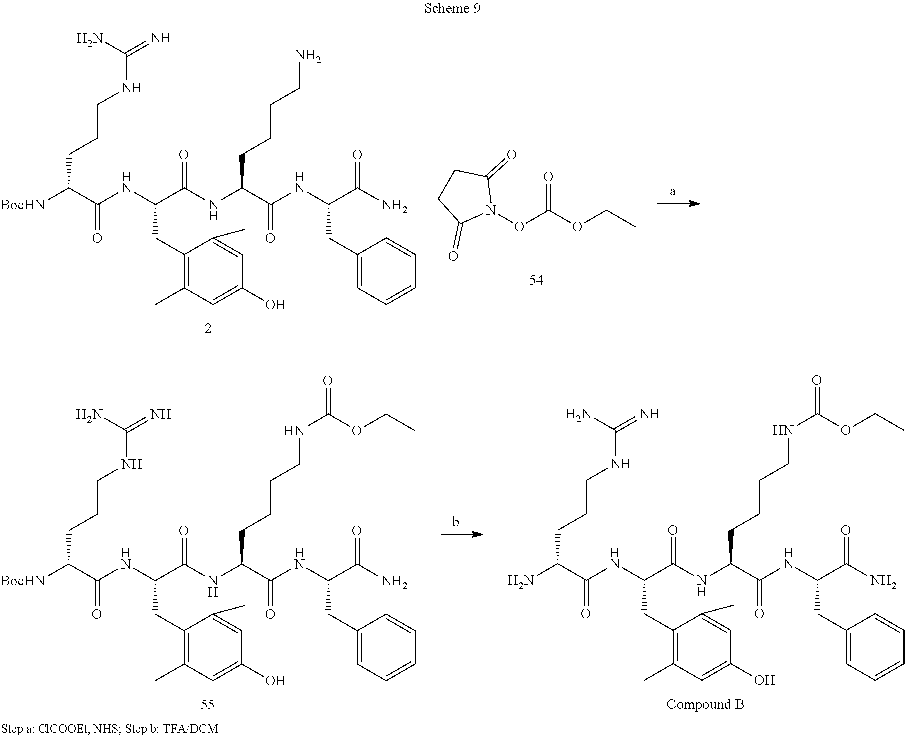

### 1) Step a: Synthesis of tert-butyl ((6R,9S,12S)-1-amino-12-(((S)-1-amino-1-oxo-3-phenylpropan-2-yl)carbamoyl)-9-(4-hydroxy-2,6-dimethylbenzyl)-1-imino-7,10,18-trioxo-19-oxa-2,8,11,17-tetraazahenicosan-6-yl)carbamate (D-(N2-Boc)-Arg-DMT-(N6-ethoxycarbonyl)-Lys-Phe-NH₂, 55)

To a mixture of 1-hydroxysuccinimide (400 mg, 3.48 mmol), ethyl chlorofomate (220 mg, 2.08 mmol), and NMM (0.527 g, 5.22 mmol) dry THF (10 mL) was added and the reaction mixture was stirred at r.t. for 2 h. Then, 2 (0.3 g, 0.35 mmol) was added and the reaction mixture was stirred at r.t. for 4 h. Crude product was purified by reversed phase flash chromatography on silica gel using a mixture of MeOH/MeCN (1:1) and 0.1% solution of AcOH in water as an eluent. The product came out of the column at 45-70% of MeOH/MeCN to give 55 (0.178 g). ¹H NMR (400 MHz, Methanol-d₄) δ 7.30-7.24 (m, 4H), 7.21-7.17 (m, 1H), 6.44 (s, 2H), 4.63 (dd, J=8.8; 6.7 Hz, 1H), 4.56 (dd, J=8.8; 5.7 Hz, 1H), 4.20-4.14 (m, 1H), 4.05 (q, J=7.1 Hz, 2H), 3.95-3.91 (m, 1H), 3.19 (dd, J=19.9; 5.7 Hz, 1H), 3.13-3.02 (m, 5H), 2.98-2.88 (m, 2H), 2.26 (s, 6H), 1.72-1.56 (m, 4H), 1.43 (s, 9H), 1.52-1.25 (m, 9H), 1.22 (t, J=7.1 Hz, 3H).

### 2) Step b: Synthesis of ethyl ((S)-6-(((S)-1-amino-1-oxo-3-phenylpropan-2-yl)amino)-5-((S)-2-((R)-2-amino-5-guanidinopentanamido)-3-(4-hydroxy-2,6-dimethylphenyl)propanamido)-6-oxohexyl)carbamate (Compound B)

To a cooled (0° C.) solution of 55 (178 mg, 3.32 mmol) in DCM (6 mL) TFA (1 mL) was added. After 15 min, the ice bath was removed and the mixture stirred at ambient temperature for 3 h. Volatiles was removed under reduced pressure. Crude product was purified by reversed phase flash chromatography using a mixture of H₂O/MeOH and 0.1% solution of TFA in water as an eluent. The product came out of the column at 35-40% of H₂O/MeOH to give Compound B (140 mg). ¹H NMR (400 MHz, Methanol-d₄) δ 7.30-7.19 (m, 5H), 6.44 (s, 2H), 4.84-4.77 (m, 1H), 4.62-4.53 (m, 1H), 4.24 (dd, J=8.2, 5.8 Hz, 1H), 4.05 (q, J=7.1 Hz, 2H), 3.90 (t, J=5.5 Hz, 1H), 3.02 (d, J=109.4 Hz, 8H), 2.27 (s, 6H), 1.77-1.16 (m, 14H). MS (M+H⁺): 712.4.

### Example 3. Synthesis of 2-((R)-2-amino-5-ethoxycarbonylguanidinopentanamido)-3-(4-hydroxy-2,6-dimethylphenyl)propanamido)-6-oxohexyl)carbamate (D-(N^{ω}-Ethoxycarbonyl)-Arg-DMT-(N6-Ethoxycarbonyl)-Lys-Phe-NH₂) (Compound C)

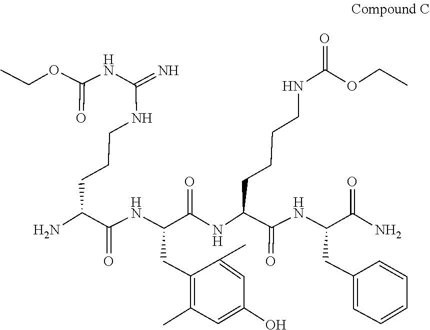 [SMILES: CCOC(=O)NCCCC[C@H](NC(=O)[C@H](Cc1c(C)cc(O)cc1C)NC(=O)[C@H](N)CCCNC(=N)NC(=O)OCC)C(=O)N[C@@H](Cc1ccccc1)C(N)=O]

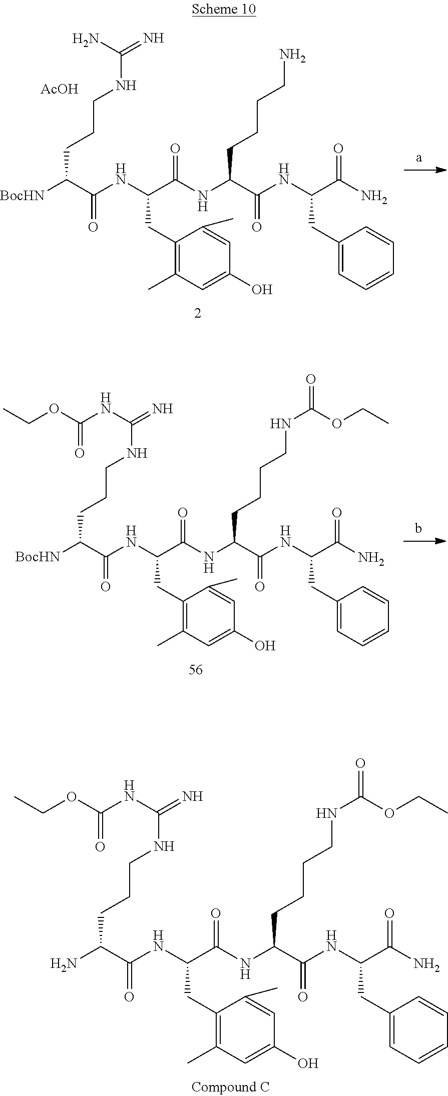

Step a: Ethyl chloroformate, pH 8.5; Step b: TFA/DCM

### 1) Step a: Synthesis of tert-butyl ((6R,9S,12S)-1-ethoxycarbonylamino-12-(((S)-1-amino-1-oxo-3-phenylpropan-2-yl)carbamoyl)-9-(4-hydroxy-2,6-dimethylbenzyl)-1-imino-7,10,18-trioxo-19-oxa-2,8,11,17-tetraazahenicosan-6-yl)carbamate (D-(N2-Boc, N^{ω}-ethoxycarbonyl)-Arg-DMT-(N6-ethoxylcarbonyl)-Lys-Phe-NH₂, 56)

To a solution of 2 (0.25 g, 0.29 mmol) in mixture of THF (10 mL) and Krebs-Ringer bicarbonate buffer (10 mL, pH 8.5, 1M) was added solution of ethyl chlorofomate (0.13 g, 1.16 mmol) in THF (5 mL). Reaction was stirred for 4 hours, then additionally was added solution ethyl chlorofomate (0.13 g, 1.16 mmol) in THF (5 mL). After additional 2 hours to reaction mixture was added AcOH (to pH 6) and reaction mixture was evaporated. Crude product was purified by reversed phase flash chromatography on silica gel using a mixture of MeOH/MeCN (1:1) and 0.1% solution of AcOH in water as an eluent. The product came out of the column at 45-70% of MeOH/MeCN to give 56 (0.135 g).

### 2) Step b: Synthesis of ethyl ((S)-6-(((S)-1-amino-1-oxo-3-phenylpropan-2-yl)amino)-5-((S)-2-((R)-2-amino-5-ethoxycarbonylguanidinopentanamido)-3-(4-hydroxy-2,6-dimethylphenyl)propanamido)-6-oxohexyl)carbamate (Example 7)

To a cooled (0° C.) solution of 56 (135 mg, 3.32 mmol) in DCM (6 mL) TFA (1 mL) was added. After 15 min, the ice bath was removed and the mixture stirred at ambient temperature for 3 h. Volatiles was removed under reduced pressure. Crude product was purified by reversed phase flash chromatography using a mixture of H₂O/MeOH and 0.1% solution of TFA in water as an eluent. The product came out of the column at 45-65% of H₂O/MeOH to give Compound C (110 mg).

¹H NMR (400 MHz, Methanol-d₄) δ 7.34-7.23 (m, 4H), 7.23-7.13 (m, 1H), 6.77 (s, 2H), 4.63-4.51 (m, 1H), 4.26 (q, J=7.1 Hz, 3H), 4.05 (q, J=7.1 Hz, 2H), 3.98-3.85 (m, 1H), 3.18-2.90 (m, 8H), 2.35 (s, 6H), 1.82-1.54 (m, 4H), 1.51-1.15 (m, 6H), 1.34 (t, J=7.1 Hz, 3H), 1.21 (t, J=7.1 Hz, 3H). MS (M+H⁺): 784.7.

### Example 4. Synthesis of 2,5,8,11,14,17,20-heptaoxadocosan-22-yl ((S)-6-(((S)-1-amino-1-oxo-3-phenylpropan-2-yl)amino)-5-((S)-2-((R)-2-amino-5-guanidinopentanamido)-3-(4-hydroxy-2,6-dimethylphenyl)propanamido)-6-oxohexyl)carbamate (D-Arg-DMT-(N6-Me(PEG)7CO)-Lys-Phe-NH₂) (Compound D)

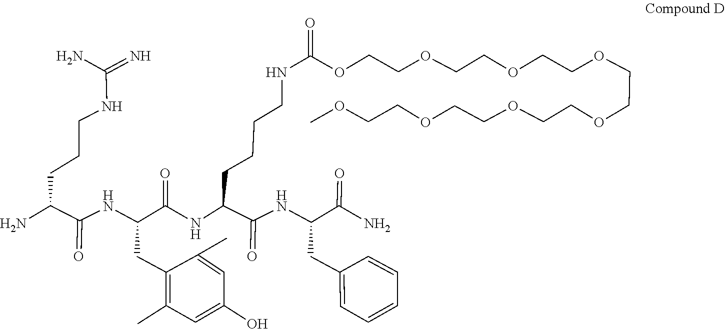 [SMILES: COCCOCCOCCOCCOCCOCCOCCOC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O]

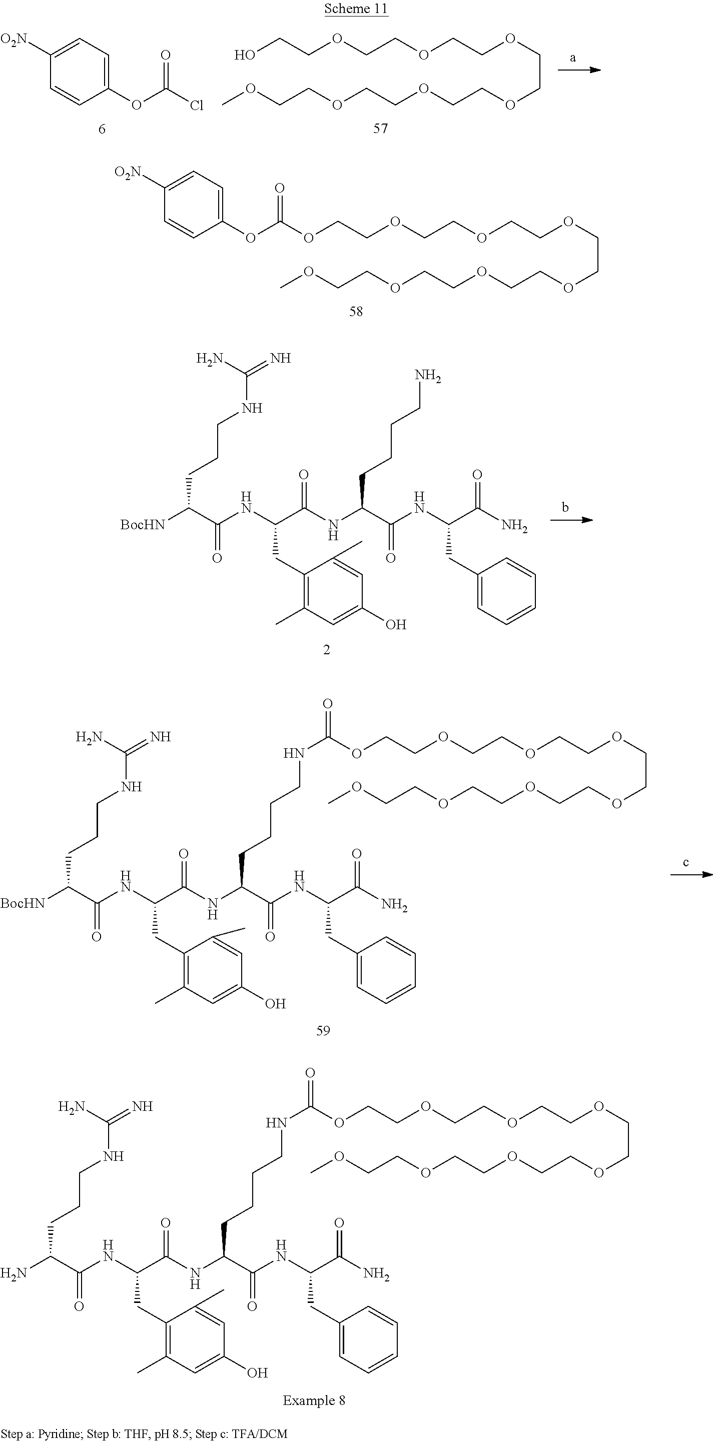

### 1) Step a: Synthesis of 2,5,8,11,14,17,20-heptaoxadocosan-22-yl (4-nitrophenyl) carbonate (58)

A stirred mixture of 4-nitrophenyl chloroformate (6, 444 mg, 2.20 mmol) and pyridine (240 μl, 2.94 mmol) in acetonitrile was allowed to cool to 0° C. for 15 min. A solution of PEG-7 (57, 500 mg, 1.47 mmol.) in acetonitrile was added slowly to the mixture. The mixture was allowed to warm to room temperature and reacted for 15 h. Then, the reaction mixture was concentrated to dryness, re-dissolved in DCM, and washed with brine. The organic layer was concentrated and dried in vacuo to give the crude product as yellow oil. The residue was chromatographed on silica gel with EtOAc/hexane (1/4 to 1/1) and then EtOAc/MeOH (9/1) as the eluent to isolate activated 58 as yellowish oil 490 g, 66%). ¹H NMR (400 MHz, Chloroform-d) δ 8.33-8.24 (m, 2H), 7.44-7.36 (m, 2H), 4.47-4.41 (m, 2H), 3.86-3.78 (m, 2H), 3.74-3.60 (m, 22H), 3.56-3.52 (m, 2H), 3.37 (s, 3H).

### 2) Step b: Synthesis of tert-butyl ((30S,33S,36R)-41-amino-30-(((S)-1-amino-1-oxo-3-phenylpropan-2-yl)carbamoyl)-33-(4-hydroxy-2,6-dimethylbenzyl)-41-imino-24,32,35-trioxo-2,5,8,11,14,17,20,23-octaoxa-25,31,34,40-tetraazahentetracontan-36-yl)carbamate (D-(N2-Boc)-Arg-DMT-(N6-Me(PEG)7CO)-Lys-Phe-NH₂, 59)

58 (0.25 g, 0.5 mmol) in THF (20 mL) was added to solution of 2 (0.4 g, 0.465 mmol) in mixture of THF (45 mL) and Krebs-Ringer bicarbonate buffer (20 mL, pH 8.5, 1M) at room temperature. Carbamate formation was completed in 2 h (monitoring with LC/MS). Reaction mixture was cooled to 0° C., acidified with AcOH to pH 5 and then evaporated to dryness (re-evaporation with toluene). The remaining mixture was used for the next step reaction without further purification.

### 3) Step c: Synthesis of Synthesis of 2,5,8,11,14,17,20-heptaoxadocosan-22-yl ((S)-6-(((S)-1-amino-1-oxo-3-phenylpropan-2-yl)amino)-5-((S)-2-((R)-2-amino-5-guanidinopentanamido)-3-(4-hydroxy-2,6-dimethylphenyl)propanamido)-6-oxohexyl)carbamate (D-Arg-DMT-(N6-Me(PEG)7CO)-Lys-Phe-NH₂, Compound D)

Remaining solid from the previous step b was suspended in DCM (120 mL) under inert atmosphere and cooled to 0° C. Afterwards, to suspension was added TFA (8 mL) and reaction allowed warming to r.t. and stirring for 3 hours. When reaction was completed solvent was evaporated and crude product was purified by reverse phase flash chromatography (eluent: H₂O (0.2% AcOH)/MeOH from 5% to 85% of methanol) to yield 460 mg of crude Compound D, which was further purified by HPLC yielding 200 mg of desired product. 41 NMR (400 MHz, Methanol-d₄) δ 7.32-7.24 (m, 4H), 7.23-7.15 (m, 1H), 6.44 (s, 2H), 4.78 (t, J=8.1 Hz, 1H), 4.58 (dd, J=8.1, 6.3 Hz, 1H), 4.27 (dt, J=8.8, 5.4 Hz, 1H), 4.19-4.12 (m, 2H), 3.93 (t, J=6.0 Hz, 1H), 3.64 (d, J=25.2 Hz, 24H), 3.54 (d, J=9.0 Hz, 2H), 3.35 (s, 3H), 3.18-2.85 (m, 8H), 2.27 (s, 6H), 1.80-1.17 (m, 10H). MS (M+H⁺): 1006.8.

### Example 5: Synthesis of (S)—N—((S)-1-amino-1-oxo-3-phenylpropan-2-yl)-2-((S)-2-((R)-2-amino-5-guanidinopentanamido)-3-(4-hydroxy-2,6-dimethylphenyl)propanamido)-6-(3-methoxypropanamido)hexanamide (Compound E)

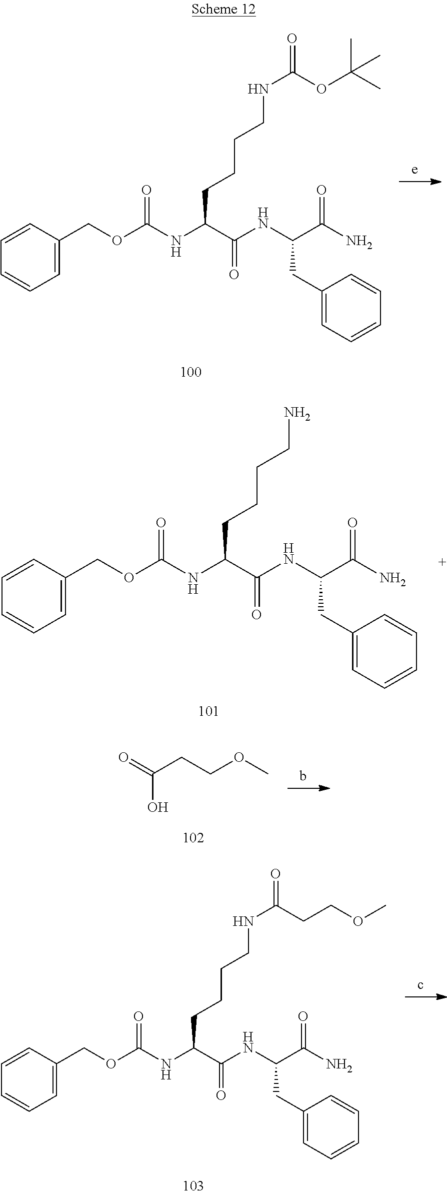

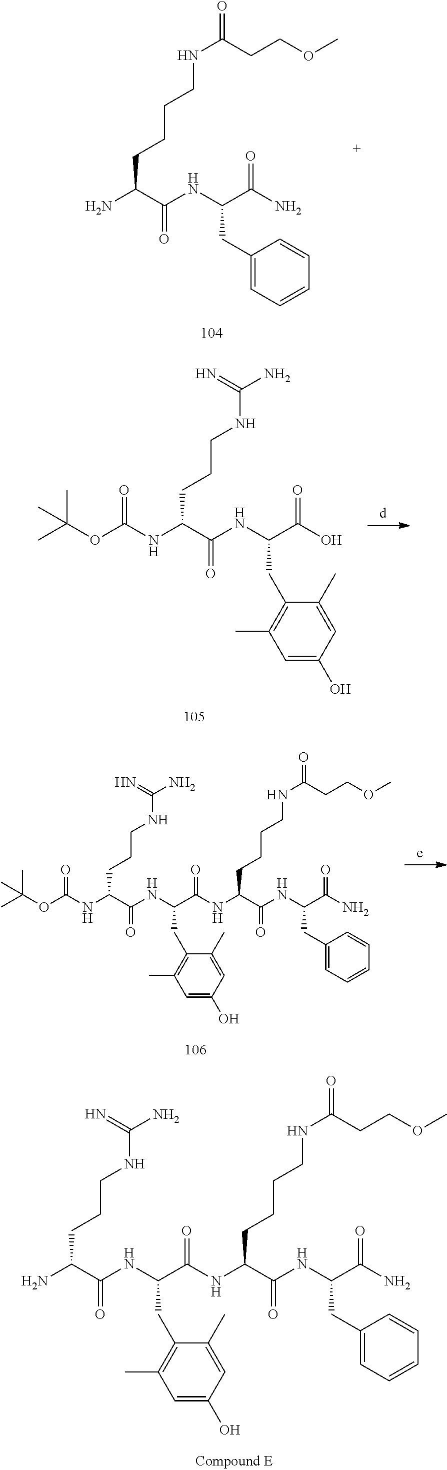 [SMILES: COCCC(=O)NCCCC[C@H](N)C(=O)N[C@@H](Cc1ccccc1)C(N)=O ; Cc1cc(O)cc(C)c1C[C@H](NC(=O)[C@@H](CCCNC(=N)N)NC(=O)OC(C)(C)C)C(=O)O ; COCCC(=O)NCCCC[C@H](NC(=O)[C@H](Cc1c(C)cc(O)cc1C)NC(=O)[C@@H](CCCNC(=N)N)NC(=O)OC(C)(C)C)C(=O)N[C@@H](Cc1ccccc1)C(N)=O ; COCCC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O]

### 1) Step a: Synthesis of Benzyl ((S)-6-amino-1-(((S)-1-amino-1-oxo-3-phenylpropan-2-yl)amino)-1-oxohexan-2-yl)carbamate (101)

To a cooled solution of benzyl tert-butyl ((S)-6-(((S)-1-amino-1-oxo-3-phenylpropan-2-yl)amino)-6-oxohexane-1,5-diyl)dicarbamate (100, 0.400 g, 0.760 mmol) in DCM (5 mL) in an ice bath was added TFA (5 mL). After 10 minutes of stirring at 0° C., ice bath was removed. After 1 h, clean conversion to 101 was observed. Volatiles were removed in vacuo. The residue was co-evaporated 2x from toluene and dried in vacuo to give the crude product that was used in the next step without further purification.

### 2) Step b: Synthesis of benzyl ((S)-1-(((S)-1-amino-1-oxo-3-phenylpropan-2-yl)amino)-6-(3-methoxypropanamido)-1-oxohexan-2-yl)carbamate (103)

To a solution of 101 (product of Step a) in N,N-Dimethylformamide (5.881 mL, 75.96 mmol) was added methoxypropanoic acid (102, 0.0870 g, 0.836 mmol), N,N,N′,N′-Tetramethyl-O-(7-azabenzotriazol-1-yl)uronium Hexafluorophosphate (0.3177 g, 0.8355 mmol) and N,N-Diisopropylethylamine (0.397 mL, 2.28 mmol). The solution turned yellow but then color faded over about 10 min. A fourth equiv of DIEA was added. The solution remained yellow. After 1h, the reaction was deemed complete. Volatiles were removed at reduced pressure and the under high vacuum. The residue was absorbed onto Celite and eluted using 10% MeOH in DCM and solvent was removed under reduced pressure. To the residue was added 30 mL of EtOH and water (60 mL). Because nothing precipitated or crystallized, the solvent was removed under reduce pressure. The reside was filtered and the solid washed with water and then diethyl ether. The residue was dried in vacuo to give 0.292 g of 103 as a white solid and was used in the next reaction without further purification.

### 3) Step c: Synthesis of (S)-2-amino-N—((S)-1-amino-1-oxo-3-phenylpropan-2-yl)-6-(3-methoxypropanamido)hexanamide (104)

To a flask containing 103 (0.287 g, 0.560 mmol) and Pd/C (10% w/w, 30 mg) was added methanol (10 mL, 200 mmol). The flask was subjected to two cycles of evacuation/back fill with H₂ and the mixture was stirred under 1 atm of H₂ at 35° C. After 2 hours, high performance liquid chromatography indicated that the starting material was consumed. The reaction was allowed to cool to r.t. and filter through Celite. The Celite pad was washed with MeOH and the combined filtrates were dried in vacuo to give 208 mg of 104 as white solid which was used in the next step without further purification.

### 4) Step d: Synthesis of tert-butyl ((11S,14S,17R)-22-amino-11-(((S)-1-amino-1-oxo-3-phenylpropan-2-yl)carbamoyl)-14-(4-hydroxy-2,6-dimethylbenzyl)-22-imino-5,13,16-trioxo-2-oxa-6,12,15,21-tetraazadocosan-17-yl)carbamate (106)

To a flask containing 104 was added (S)-2-((R)-2-((tert-butoxycarbonyl)amino)-5-guanidinopentanamido)-3-(4-hydroxy-2,6-dimethylphenyl)propanoic acid (105, 0.3092 g, 0.6159 mmol), isopropyl alcohol (3 mL, 40 mmol) and 1-Hydroxybenzotriazole (0.01940 g, 0.1120 mmol). The mixture stirred for several minutes without dissolution and then warmed to 40° C. for a couple of minutes. The solids did not dissolve so the flask was briefly sonicated. The solids are still not completely dissolved so DCM (3 mL) was added. After a couple of minutes, the solids dissolved. The flask was then partially concentrated to remove DCM. Because everything remained in solution, the flask was cool to 0° C. Everything remained in solution so N-(3-Dimethylaminopropyl)-N′-ethylcarbodiimide hydrochloride (0.1181 g, 0.6159 mmol) was added. After 5 minutes, the ice bath was removed and the reaction stirred at r.t. overnight. HPLC indicated good conversion to product. Several small peaks were observed near product peak. The reaction mixture was then concentrated and the residue was dissolved in DMF and purified by RPCF; followed by purification by flash chromatography. Fractions were pooled, partially concentrated and then lyophilized to give 0.335 g of 106 as a white powder.

### 5) Step e: Synthesis of (S)—N—((S)-1-amino-1-oxo-3-phenylpropan-2-yl)-2-((S)-2-((R)-2-amino-5-guanidinopentanamido)-3-(4-hydroxy-2,6-dimethylphenyl)propanamido)-6-(3-methoxypropanamido)hexanamide (Compound E)

To a cooled (0° C.) suspension of 106 (0.330 g, 0.351 mmol) in DCM (5 mL) was slowly added TFA (5 mL). After 5 min at 0° C., the ice bath was removed and the solution stirred at r.t. for 1 hour. HPLC analysis confirmed that the starting material was consumed. Volatiles were removed at reduced pressure and the residue was dissolved in DMF (5 mL). This was purified by flash chromatography. Pure fractions were combined, partially concentrated and lyophilized to afford 309 mg of 107 as a white powder. H-NMR and mass spectrometer analysis was consistent with the expected product.

### Example 6: Synthesis of (S)—N—((S)-1-amino-1-oxo-3-phenylpropan-2-yl)-2-((S)-2-((R)-2-amino-5-guanidinopentanamido)-3-(4-hydroxy-2,6-dimethylphenyl)propanamido)-6-(3-(2-(2-methoxyethoxy)ethoxy)propanamido)hexanamide (Compound F)

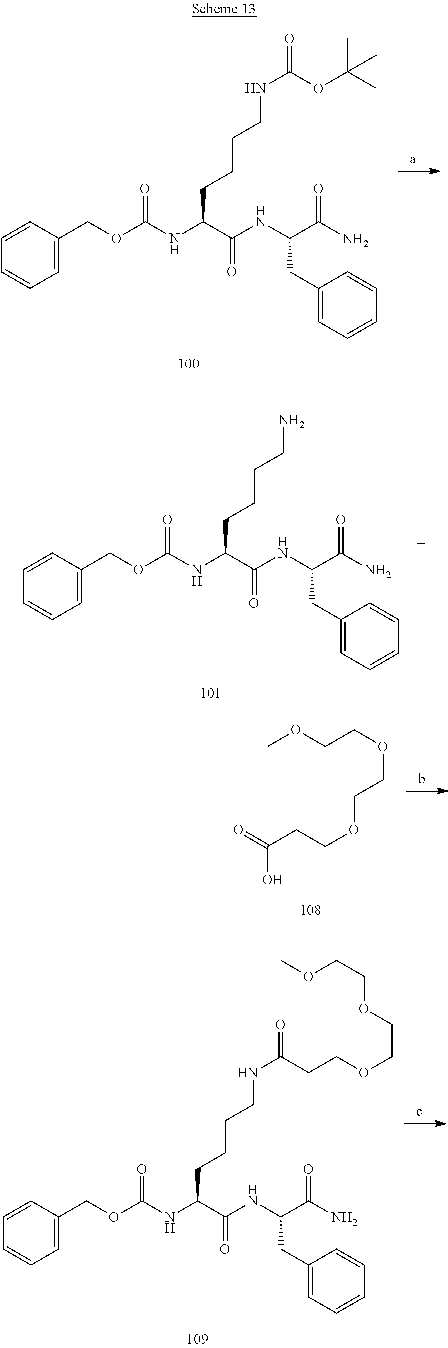 [SMILES: CC(C)(C)OC(=O)NCCCC[C@H](NC(=O)OCc1ccccc1)C(=O)N[C@@H](Cc1ccccc1)C(N)=O ; NCCCC[C@H](NC(=O)OCc1ccccc1)C(=O)N[C@@H](Cc1ccccc1)C(N)=O ; COCCOCCOCCC(=O)O ; COCCOCCOCCC(=O)NCCCC[C@H](NC(=O)OCc1ccccc1)C(=O)N[C@@H](Cc1ccccc1)C(N)=O]

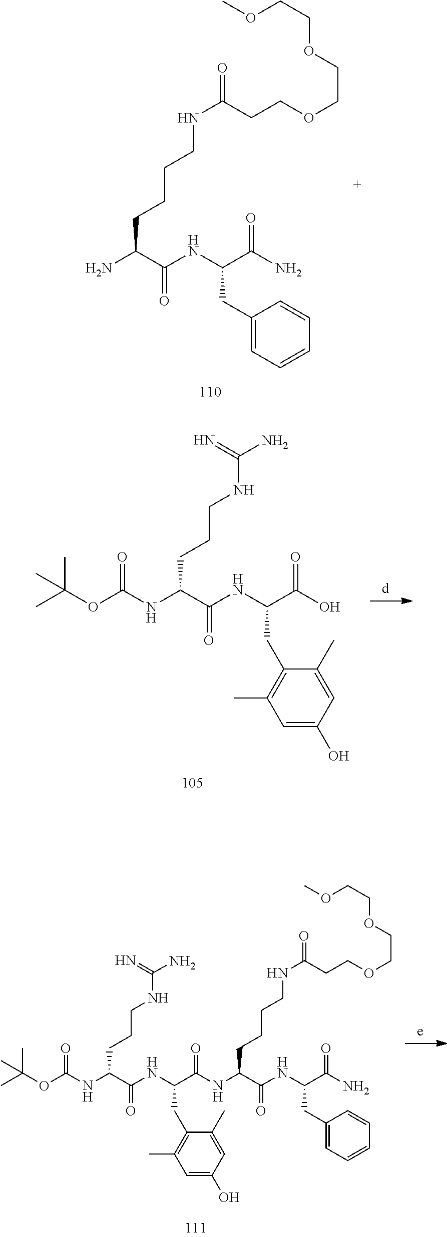 [SMILES: COCCOCCOCCC(=O)NCCCC[C@H](N)C(=O)N[C@@H](Cc1ccccc1)C(N)=O ; Cc1cc(O)cc(C)c1C[C@H](NC(=O)[C@@H](CCCNC(=N)N)NC(=O)OC(C)(C)C)C(=O)O ; COCCOCCOCCC(=O)NCCCC[C@H](NC(=O)[C@H](Cc1c(C)cc(O)cc1C)NC(=O)[C@@H](CCCNC(=N)N)NC(=O)OC(C)(C)C)C(=O)N[C@@H](Cc1ccccc1)C(N)=O]

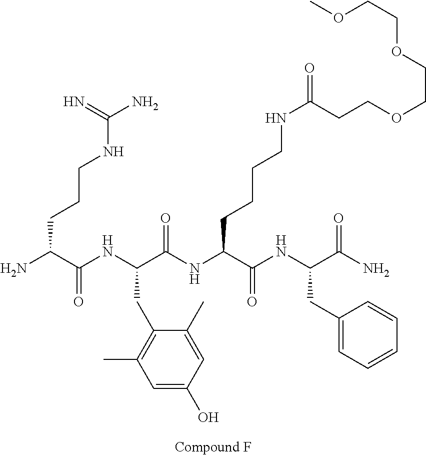 [SMILES: COCCOCCOCCC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O]

### 1) Step a: Synthesis of benzyl ((S)-6-amino-1-(((S)-1-amino-1-oxo-3-phenylpropan-2-yl)amino)-1-oxohexan-2-yl)carbamate (101)

To a solution of 100 (0.600 g, 1.14 mmol) in DCM (6 mL) was added 4 M HCl in 1,4-Dioxane (2.848 mL, 11.39 mmol) at r.t. The reaction was stirred under a dry atmosphere. After several minutes a precipitate formed. After 2 hours, HPLC indicated a slight peak for remaining starting material. After an additional 1 hour of stirring the reaction was deemed complete. The reaction mixture was diluted with DCM to provide a better mixture and the concentrated under reduced pressure. The reside (101) was dried in vacuo and used in the next reaction without further purification.

### 2) Step b: Synthesis of benzyl ((175,205)-21-amino-20-benzyl-11,18,21-trioxo-2,5,8-trioxa-12,19-diazahenicosan-17-yl)carbamate (109)

The product of step b (101) and 3-[2-(2-methoxyethoxy)ethoxy]propanoic acid (108, 0.2628 g, 1.367 mmol) was dissolved in N,N-Dimethylformamide (7.057 mL, 91.15 mmol). To this mixture as added N,N,N′,N′-Tetramethyl-O-(7-azabenzotriazol-1-yl)uronium Hexafluorophosphate (0.4765 g, 1.253 mmol) and N,N-Diisopropylethylamine (0.595 mL, 3.42 mmol). The resulting yellow solution was stirred at r.t. After stirring overnight, the starting material was consumed. The mixture was then placed under partial vacuum to remove any excess base. Acetic acid (0.2591 mL, 4.557 mmol) was added and the product was subjected to DMF solution and to RPCF. The resulting product was purified by flash chromatography and the fractions were combined and partially concentrated. The partial concentrate was extracted with 20% TFE in DCM. The combined organic layers were washed with brine, dried, filtered and evaporated to give 0.541 g of 109 as a white solid.

### 3) Step c: Synthesis of (S)-2-amino-N—((S)-1-amino-1-oxo-3-phenylpropan-2-yl)-6-(3-(2-(2-methoxyethoxy)ethoxy)propanamido)hexanamide (110)

To a flask containing 109 (0.541g, 0.901 mmol) was added Pd/C (50 mg, 10% w/w) followed by MeOH (50 ml). The flask was subjected to two cycles of evacuation/back fill with H₂. The mixture was stirred at 35° C. and 1 atm H₂ for 3 h. The mixture was cooled and filtered through Celite and washed with additional methanol. Volatiles were removed in vacuo to afford 110 as a colorless solid.

### 4) Step d: Synthesis of tert-butyl ((17S,20S,23R)-28-amino-17(((S)-1-amino-1-oxo-3-phenylpropan-2-yl)carbamoyl)-20-(4-hydroxy-2,6-dimethylbenzyl)-28-imino-11,19,22-trioxo-2,5,8-trioxa-12,18,21,27-tetraazaoctacosan-23-yl)carbamate (111)

To a mixture of 110 and (S)-2-((R)-2-((tert-butoxycarbonyl)amino)-5-guanidinopentanamido)-3-(4-hydroxy-2,6-dimethylphenyl)propanoic acid (105, 0.4973 g, 0.9907 mmol) in isopropyl alcohol (5.4 mL, 7.0E1 mmol) was added DCM (10 mL) with vigorous stirring. After 10 minutes, the solution was partially concentrated at reduced pressure to remove the added DCM and cooled (0° C.). To the cooled solution was added 1-Hydroxybenzotriazole (0.0312 g, 0.180 mmol) followed by EDC·HCl (0.190 g, 0.991 mmol). After 10 minutes, the ice bath was removed and the reaction stirred at room temperature overnight. Volatiles were removed at reduced pressure and the residue purified by RPCF chromatography. Fractions were combined and concentrated to give 0.680 g of 111 as a white powder.

### 5) Step e: Synthesis of (S)—N—((S)-1-amino-1-oxo-3-phenylpropan-2-yl)-2-((S)-2-((R)-2-amino-5-guanidinopentanamido)-3-(4-hydroxy-2,6-dimethylphenyl)propanamido)-6-(3-methoxypropanamido)hexanamide (Compound F)

To a cooled (0° C.) mixture of 111 (0.680 g, 0.661 mmol) in DCM (10 mL) was slowly added TFA (10 mL). After 10 minutes the ice bath was removed and the reaction was allowed to stir at r.t. for 1 hour. HPLC analysis of the reaction indicated clean conversion of starting material to product but identified one late eluting impurity. Volatiles were removed reduced pressure and the residue was dissolved in DMF (5 mL). This solution was purified by flash chromatography. Pure fractions were combined, partially concentrated and lyophilized to afford 565 mg of 112 as a white powder. H-NMR and mass spectrometer analysis was consistent with the expected product.

### Example 7: D-Arg-Dmt-Lys-Phe-NH₂ (SS-31) can Protect Against MPT, Mitochondrial Swelling and Cytochrome c Release

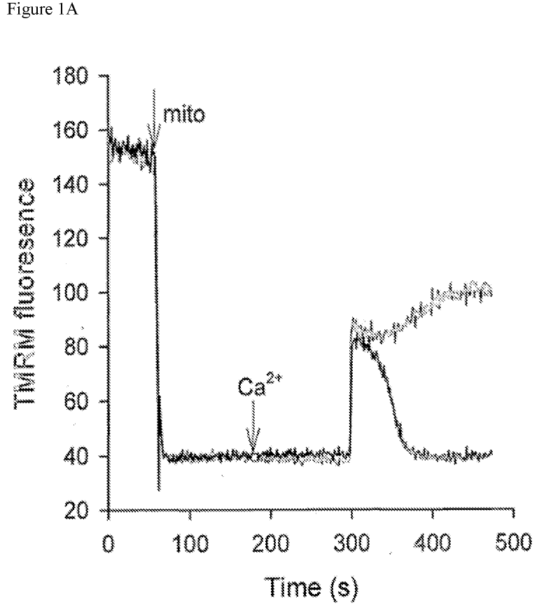

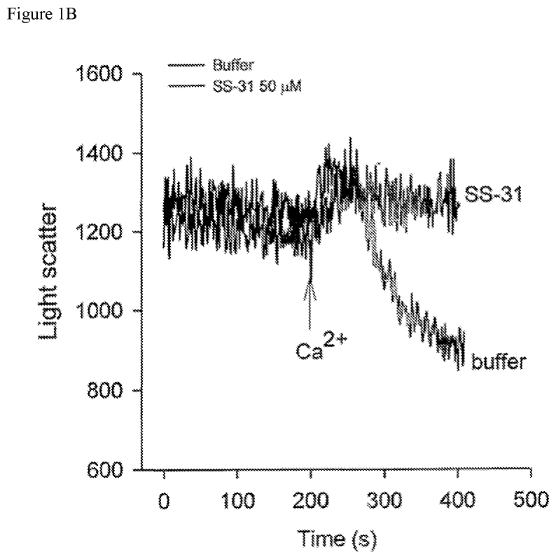

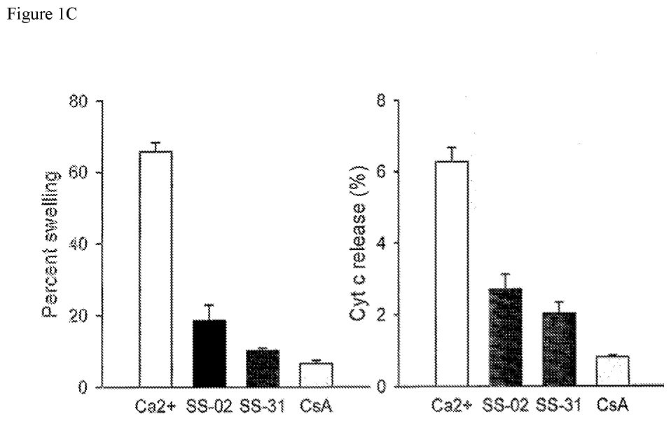

The non-opioid peptide SS-31 has the same ability to protect against MPT (FIG. 1A), mitochondrial swelling (FIG. 1B), and cytochrome c release (FIG. 1C), induced by ca2+. MPT pore opening results in mitochondrial swelling. We examined the effects of (SS-31) on mitochondrial swelling, which was measured using light scattering monitored at 570 nm.

### Example 8: 2′,6′-Dmt-D-Arg-PheLys-NH₂ (SS-02) and D-Arg-Dmt-Lys-Phe-NH₂ (SS-31) Protects Against Ischemia-Reperfusion-Induced Myocardial Stunning

Guinea pig hearts were rapidly isolated, and the aorta was cannulated in situ and perfused in a retrograde fashion with an oxygenated Krebs-Henseleit solution (pH 7.4) at 34° C. The heart was then excised, mounted on a modified Langendorff perfusion apparatus, and perfused at constant pressure (40 cm H₂0). Contractile force was measured with a small hook inserted into the apex of the left ventricle and the silk ligature tightly connected to a force-displacement transducer. Coronary flow was measured by timed collection of pulmonary artery effluent.

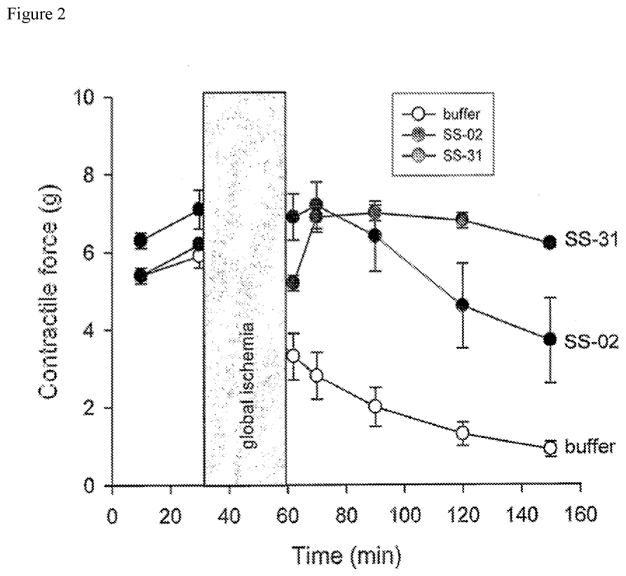

Hearts were perfused with buffer, 2′,6′-Dmt-D-Arg-PheLys-NH₂ (SS-02) (100 nM) or D-Arg-Dmt-Lys-Phe-NH₂ (SS-31) (1 nM) for 30 min and then subjected to 30 min of global ischemia Reperfusion was carried out with the same solution used prior to ischemia. Two-way ANOVA revealed significant differences in contractile force (P<0.001), heart rate (P=0.003), and coronary flow (P<0.001) among the three treatment groups. In the buffer group, contractile force was significantly lower during reperfusion compared with before ischemia (FIG. 2). Both SS-02 and SS-31 treated hearts tolerated ischemia much better than buffer-treated hearts (FIG. 2). In particular, SS-31 provided complete inhibition of cardiac stunning. In addition, coronary flow is well-sustained throughout reperfusion and there was no decrease in heart rate.

### INCORPORATION BY REFERENCE

All of the U.S. patents and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A compound of Formula (I) 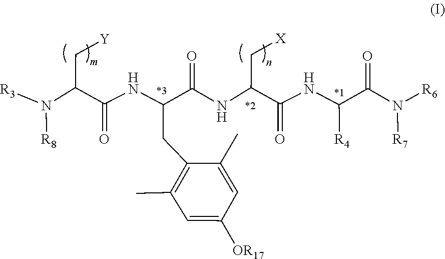
wherein:
X is —N(R₁₅)—R₁, 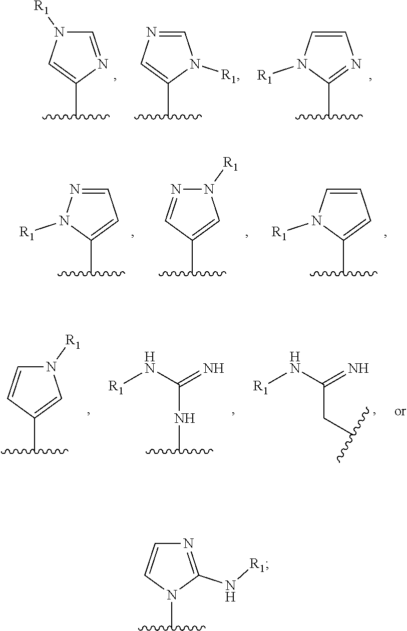
Y is —N(R₁₅)—R₂, 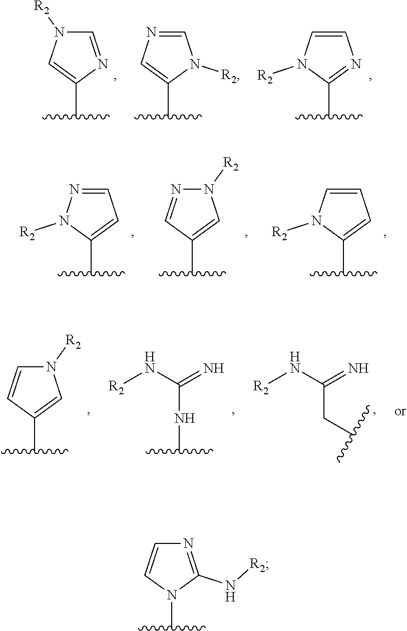
R₁, R₂, R₃, and R₁₇ are independently H, alkyl, alkenyl, alkynyl, aryl, arylalkyl, arylheteroalkyl, cycloalkyl, heteroalkyl, heteroaryl, T, R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₁O)(R₁₂O)P(O)—, or R₁₁R₁₂N(R₉O)P(O)—;
R₄ is alkyl, cycloalkyl, aryl, arylalkyl, heteroaryl, arylheteroalkyl, T, a side-chain of a naturally or non-naturally occurring chiral amino acid, 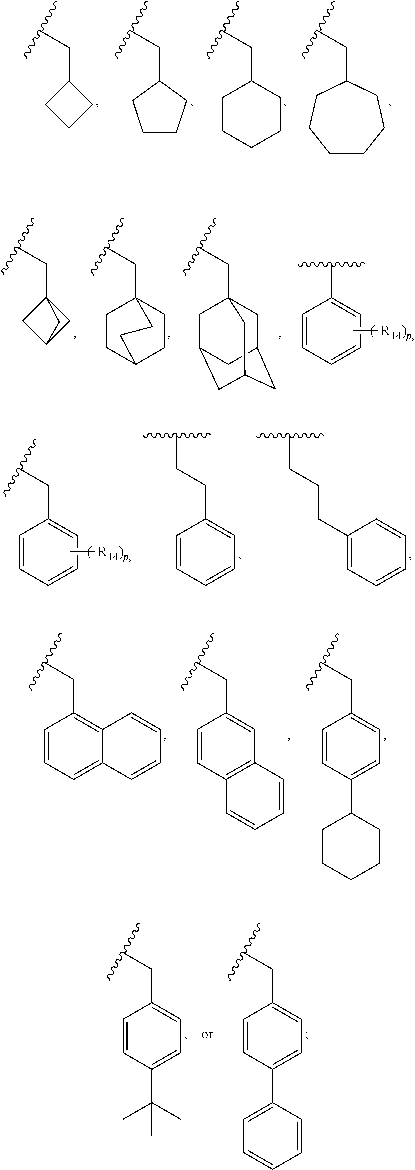
R₆ and R₇ are independently H, alkyl, or acyl; or R₆ and R₇ together with the nitrogen atom to which they are attached form a 4-6-membered heterocyclic ring;
R₈ is H, alkyl, heteroalkyl, or acyl;
R₉, R₁₁, and R₁₂ are independently H, alkyl, alkenyl, alkynyl, heteroalkyl, cycloalkyl, aryl, arylalkyl, heteroaryl, arylheteroalkyl, heteroarylheteroalkyl, or T;
R₁₁ and R₁₂ can be taken together to form a heterocyclic ring;
R₁₀ is alkyl, alkenyl, alkynyl, heteroalkyl, cycloalkyl, aryl, arylalkyl, heteroaryl, arylheteroalkyl, heteroarylheteroalkyl, or T;
R₁₃ is H, methyl, ethyl, isopropyl, or tert-butyl;
R₁₄ is independently D, F, Cl, Br, I, —CH₃, —OCH₃, CH₂CH₃, —OCH₂CH₃, —CCl₃, —CF₃, —C≡N, —OH, or —NO₂;
T is —(CH₂)_{w}—(O)ₓ—[(CH₂CH₂)—O]_{q}—R₁₃;
n and m are independently 1, 2, 3, 4, 5, or 6;
p is 0, 1, 2, 3, 4, or 5;
q is an integer from 1-30 inclusive;
x is 0 or 1; and
w is 0, 1 or 2; provided that: if x is 0 then w is 0; and if w is 0, then x is 0;
the absolute stereochemistry at each of stereocenters 0.1, 0.2, *3 and *4 is independently R (D for an amino acid) or S (L for an amino acid); and
at least one of R₁, R₂, R₃ and R₁₇ is R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₁O)(R₁₂O)P(O)—, or R₁₁R₁₂N(R₉O)P(O)—.

2. The compound of claim 1, wherein each of R₁, R₂, R₃ and R₁₇ is not: Cbz, Boc, Bpoc, Bhoc, Nps, Bpoc, Ddz, Fmoc, ivDde, Msc, Nsc, Bsmoc, Sps, or Esc.

3. The compound of claim 1 or 2, wherein X is —N(R₁₅)—R₁.

4. The compound of claim 1 or 2, wherein X is

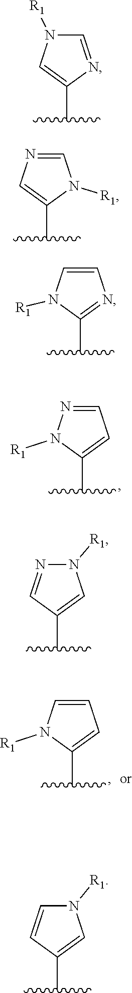

5. The compound of claim 1 or 2, wherein X is

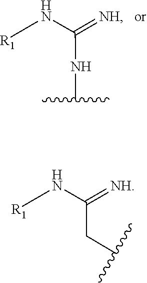

6. The compound of claim 1 or 2, wherein X is

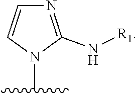

7. The compound of any one of claims 1-6, wherein Y is —N(R₁₅)—R₂.

8. The compound of any one of claims 1-6, wherein Y is

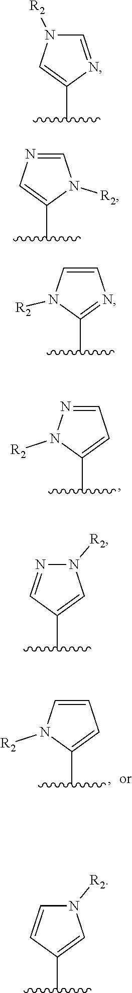

9. The compound of any one of claims 1-6, wherein Y is

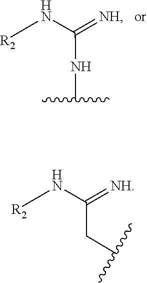

10. The compound of any one of claims 1-6, wherein Y is

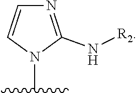

11. The compound of any one of claims 1-10, wherein R₁ is H.

12. The compound of any one of claims 1-10, wherein R₁ is alkyl, alkenyl, alkynyl, aryl, arylalkyl, arylheteroalkyl, cycloalkyl, heteroalkyl, or heteroaryl.

13. The compound of any one of claims 1-10, wherein R₁ is T.

14. The compound of claim 13, wherein R₁ is —[(CH₂CH₂)—O]_{q}—R₁₃.

15. The compound of any one of claims 1-10, wherein R₁ is R₉C(O)—, R₁₀OC(O)—, or (R₁₁O)(R₁₂O)P(O)—.

16. The compound of any one of claims 1-10, wherein R₁ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—.

17. The compound of any one of claims 1-16, wherein R₂ is H.

18. The compound of any one of claims 1-16, wherein R₂ is alkyl, alkenyl, alkynyl, aryl, arylalkyl, arylheteroalkyl, cycloalkyl, heteroalkyl, or heteroaryl.

19. The compound of any one of claims 1-16, wherein R₂ is T.

20. The compound of claim 19, wherein R₂ is —[(CH₂CH₂)—O]_{q}—R₁₃.

21. The compound of any one of claims 1-16, wherein R₂ is R₉C(O)—, R₁₀OC(O)—, or (R₁₁O)(R₁₂O)P(O)—.

22. The compound of any one of claims 1-16, wherein R₂ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—.

23. The compound of any one of claims 1-22, wherein R₃ is H.

24. The compound of any one of claims 1-22, wherein R₃ is alkyl, alkenyl, alkynyl, aryl, arylalkyl, arylheteroalkyl, cycloalkyl, heteroalkyl, or heteroaryl.

25. The compound of any one of claims 1-22, wherein R₃ is T.

26. The compound of claim 25, wherein R₃ is —[(CH₂CH₂)—O]_{q}—R₁₃.

27. The compound of any one of claims 1-22, wherein R₃ is R₉C(O)—, R₁₀OC(O)—, or (R₁₁O)(R₁₂O)P(O)—.

28. The compound of any one of claims 1-22, wherein R₃ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—.

29. The compound of any one of claims 1-28, wherein R₁₇ is H.

30. The compound of any one of claims 1-28, wherein R₁₇ is alkyl, alkenyl, alkynyl, aryl, arylalkyl, arylheteroalkyl, cycloalkyl, heteroalkyl, or heteroaryl.

31. The compound of any one of claims 1-28, wherein R₁₇ is T.

32. The compound of claim 31, wherein R₁₇ is —[(CH₂CH₂)—O]_{q}—R₁₃.

33. The compound of any one of claims 1-28, wherein R₁₇ is R₉C(O)—, R₁₀OC(O)—, or (R₁₁O)(R₁₂O)P(O)—.

34. The compound of any one of claims 1-28, wherein R₁₇ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—.

35. The compound of any one of claims 1-34, wherein R₄ is alkyl, cycloalkyl, aryl, arylalkyl, heteroaryl, or arylheteroalkyl.

36. The compound of any one of claims 1-34, wherein R₄ is T.

37. The compound of claim 36, wherein R₄ is —(CH₂)—(O)—[(CH₂CH₂)—O]_{q}—R₁₃.

38. The compound of claim 36, wherein R₄ is —(CH₂)₂—(O)—[(CH₂CH₂)—O]_{q}—R₁₃.

39. The compound of any one of claims 1-34, wherein R₄ is a side-chain of a naturally or non-naturally occurring chiral amino acid.

40. The compound of any one of claims 1-34, wherein R₄ is

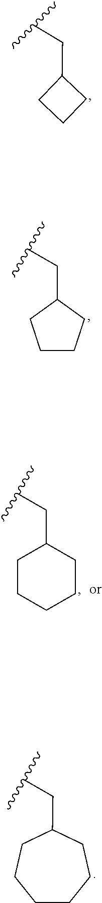 [SMILES: *CC1CCC1 ; *CC1CCCC1 ; *CC1CCCCC1 ; *CC1CCCCCC1]

41. The compound of any one of claims 1-34, wherein R₄ is

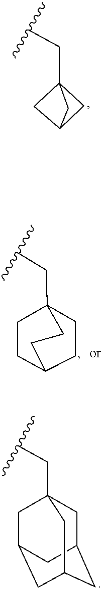 [SMILES: *CC12CC(C1)C2 ; *CC12CCC(CC1)CC2 ; *CC12CC3CC(CC(C3)C1)C2]

42. The compound of any one of claims 1-34, wherein R₄ is

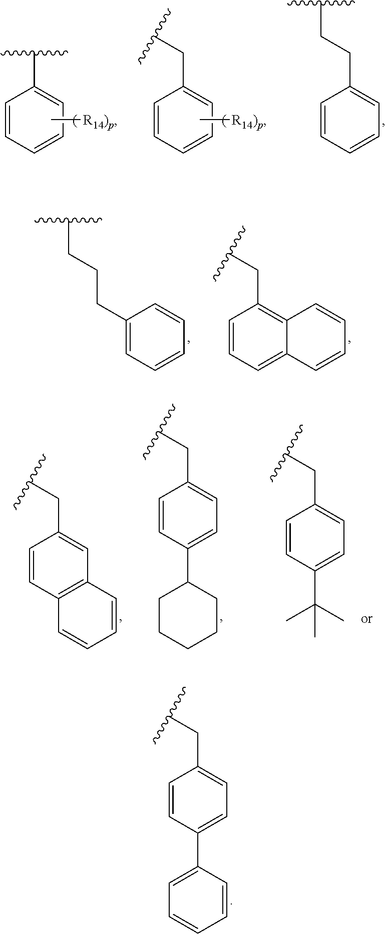

43. The compound of claim 42, wherein R₄ is 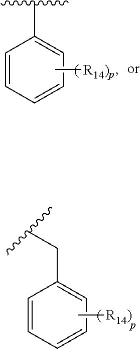
and each R₁₄ is H.

44. The compound of any one of claims 1-43, wherein R₆ is H.

45. The compound of any one of claims 1-43, wherein R₆ is alkyl or acyl.

46. The compound of any one of claims 1-45, wherein R₇ is H.

47. The compound of any one of claims 1-45, wherein R₇ is alkyl or acyl.

48. The compound of any one of claims 1-43, wherein R₆ and R₇ together with the nitrogen atom to which they are attached form a 4-6-membered heterocyclic ring.

49. The compound of any one of claims 1-48, wherein R₈ is H.

50. The compound of any one of claims 1-48, wherein R₈ is alkyl, heteroalkyl, or acyl.

51. The compound of any one of claims 1-48, wherein R₈ is H, methyl or ethyl.

52. The compound of any one of claims 1-51, wherein R₉ is H.

53. The compound of any one of claims 1-51, wherein R₉ is alkyl, alkenyl, alkynyl, heteroalkyl, cycloalkyl, aryl, arylalkyl, heteroaryl, arylheteroalkyl, or heteroarylheteroalkyl.

54. The compound of claim 53, wherein R₉ is C₁-C₈ alkyl.

55. The compound of any one of claims 1-51, wherein R₉ is T.

56. The compound of claim 55, wherein R₉ is —[(CH₂CH₂)—O]_{q}—R₁₃ and q is 1-20.

57. The compound of any one of claims 1-56, wherein R₁₀ is alkyl, alkenyl, alkynyl, heteroalkyl, cycloalkyl, aryl, arylalkyl, heteroaryl, arylheteroalkyl, or heteroarylheteroalkyl.

58. The compound of claim 57, wherein R₁₀ is C₁-C₈ alkyl.

59. The compound of any one of claims 1-56, wherein R₁₀ is T.

60. The compound of claim 59, wherein R₁₀ is —[(CH₂CH₂)—O]_{q}—R₁₃ and q is 1-20.

61. The compound of any one of claims 1-56, wherein R₁₁ is H.

62. The compound of any one of claims 1-56, wherein R₁₁ is alkyl, alkenyl, alkynyl, heteroalkyl, cycloalkyl, aryl, arylalkyl, heteroaryl, arylheteroalkyl, or heteroarylheteroalkyl.

63. The compound of claim 62, wherein R₁₁ is C₁-C₈ alkyl.

64. The compound of any one of claims 1-56, wherein R₁₁ is T.

65. The compound of claim 64, wherein R₁₁ is —[(CH₂CH₂)—O]_{q}—R₁₃ and q is 1-20.

66. The compound of any one of claims 1-65, wherein R₁₂ is H.

67. The compound of any one of claims 1-65, wherein R₁₂ is alkyl, alkenyl, alkynyl, heteroalkyl, cycloalkyl, aryl, arylalkyl, heteroaryl, arylheteroalkyl, or heteroarylheteroalkyl.

68. The compound of claim 67, wherein R₁₂ is C₁-C₈ alkyl.

69. The compound of any one of claims 1-65, wherein R₁₂ is T.

70. The compound of claim 69, wherein R₁₂ is —[(CH₂CH₂)—O]_{q}—R₁₃ and q is 1-20.

71. The compound of any one of claims 1-70, wherein R₁₃ is H.

72. The compound of any one of claims 1-70, wherein R₁₃ is methyl, ethyl, isopropyl, or tert-butyl.

73. The compound of any one of claims 1-42 and 44-72, wherein R₁₄ is D.

74. The compound of any one of claims 1-42 and 44-72, wherein R₁₄ is F, Cl, Br, I, —CCl₃, or —CF₃.

75. The compound of any one of claims 1-42 and 44-72, wherein R₁₄ is —CH₃, —OCH₃, CH₂CH₃, —OCH₂CH₃, —OH, or —NO₂.

76. The compound of any one of claims 1-3 and 7, wherein R₁₅ is H.

77. The compound of any one of claims 1-3 and 7, wherein R₁₅ is alkyl, alkenyl, alkynyl, cycloalkyl, heteroalkyl, or acyl.

78. The compound of claim 77, wherein R₁₅ is methyl, ethyl, isopropyl or tert-butyl.

79. The compound of any one of claims 1-78, wherein R₁₇ is H.

80. The compound of any one of claims 1-78, wherein R₁₇ is alkyl, alkenyl, alkynyl, aryl, arylalkyl, arylheteroalkyl, cycloalkyl, heteroalkyl, or heteroaryl.

81. The compound of any one of claims 1-78, wherein R₁₇ is —(CH₂)—(O)—[(CH₂CH₂)—O]_{q}—R₁₃ or —(CH₂)₂—(O)—[(CH₂CH₂)—O]_{q}—R₁₃.

82. The compound of any one of claims 1-78, wherein R₁₇ is R₉C(O)—, R₁₀OC(O)—, or (R₁₁O)(R₁₂O)P(O)—.

83. The compound of any one of claims 1-78, wherein R₁₇ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—.

84. The compound of any one of claims 1-83, wherein n is 1, 2, 3 or 4.

85. The compound of any one of claims 1-83, wherein n is 5, or 6.

86. The compound of any one of claims 1-83, wherein m is 1, 2, 3 or 4.

87. The compound of any one of claims 1-83, wherein m is 5, or 6.

88. The compound of any one of claims 1-87, wherein the stereochemistry at the carbon atom labeled *4 is D, the stereochemistry at the carbon atom labeled *3 is L, the stereochemistry at the carbon atom labeled *2 is L, and the stereochemistry at the carbon atom labeled *1 is L.

89. The compound of any one of claims 1-87, wherein the stereochemistry at the carbon atom labeled *4 is L, the stereochemistry at the carbon atom labeled *3 is D, the stereochemistry at the carbon atom labeled *2 is D, and the stereochemistry at the carbon atom labeled *1 is D.

90. The compound of any one of claims 1-87, wherein the stereochemistry at the carbon atom labeled *4 is D, the stereochemistry at the carbon atom labeled *3 is D, the stereochemistry at the carbon atom labeled *2 is D, and the stereochemistry at the carbon atom labeled *1 is D.

91. The compound of any one of claims 1-87, wherein the stereochemistry at the carbon atom labeled *4 is L, the stereochemistry at the carbon atom labeled *3 is L, the stereochemistry at the carbon atom labeled *2 is L, and the stereochemistry at the carbon atom labeled *1 is L.

92. The compound of any one of claims 1-87, wherein the stereochemistry at the carbon atom labeled *4 is D, the stereochemistry at the carbon atom labeled *3 is L, the stereochemistry at the carbon atom labeled *2 is D, and the stereochemistry at the carbon atom labeled *1 is L.

93. The compound of any one of claims 1-87, wherein the stereochemistry at the carbon atom labeled *4 is L, the stereochemistry at the carbon atom labeled *3 is D, the stereochemistry at the carbon atom labeled *2 is L, and the stereochemistry at the carbon atom labeled *1 is D.

94. The compound of claim 1, wherein the compound is

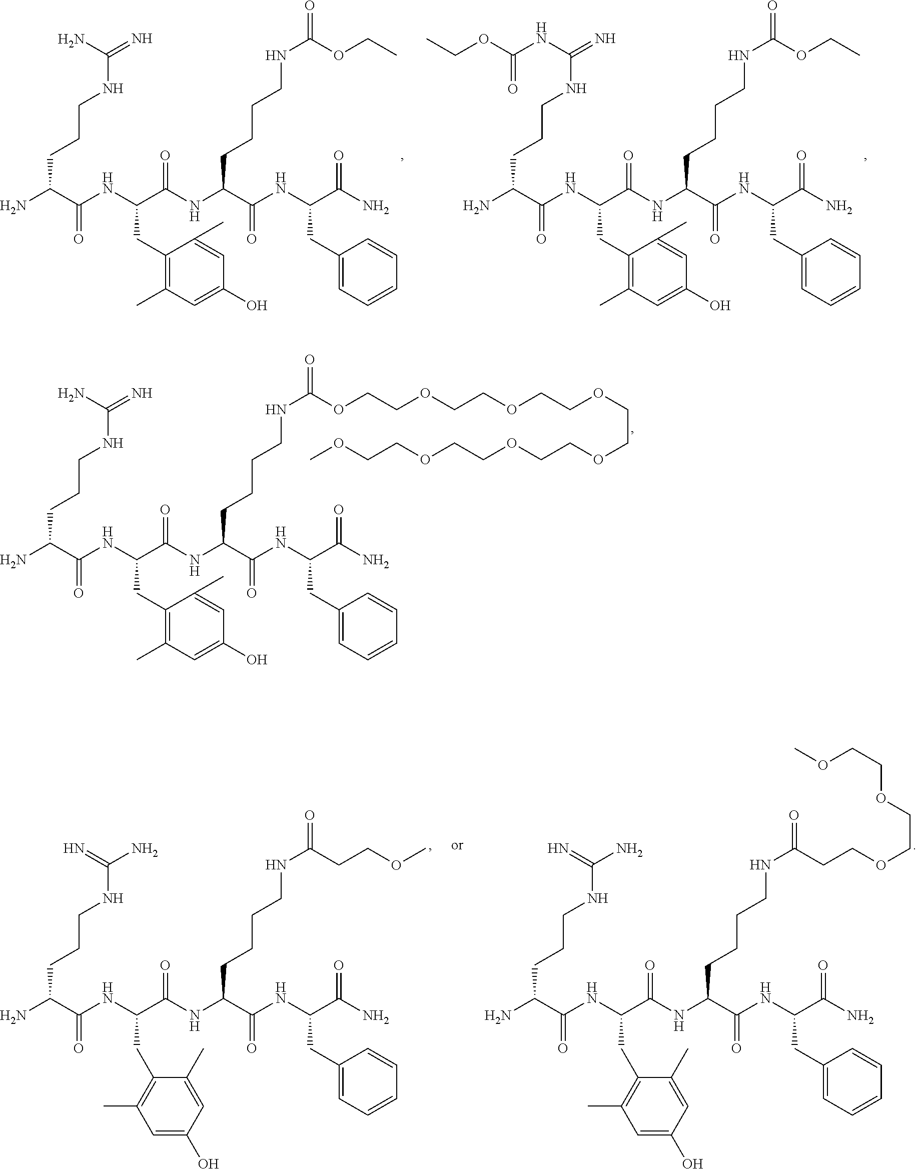

95. The compound of claim 1, wherein the compound is

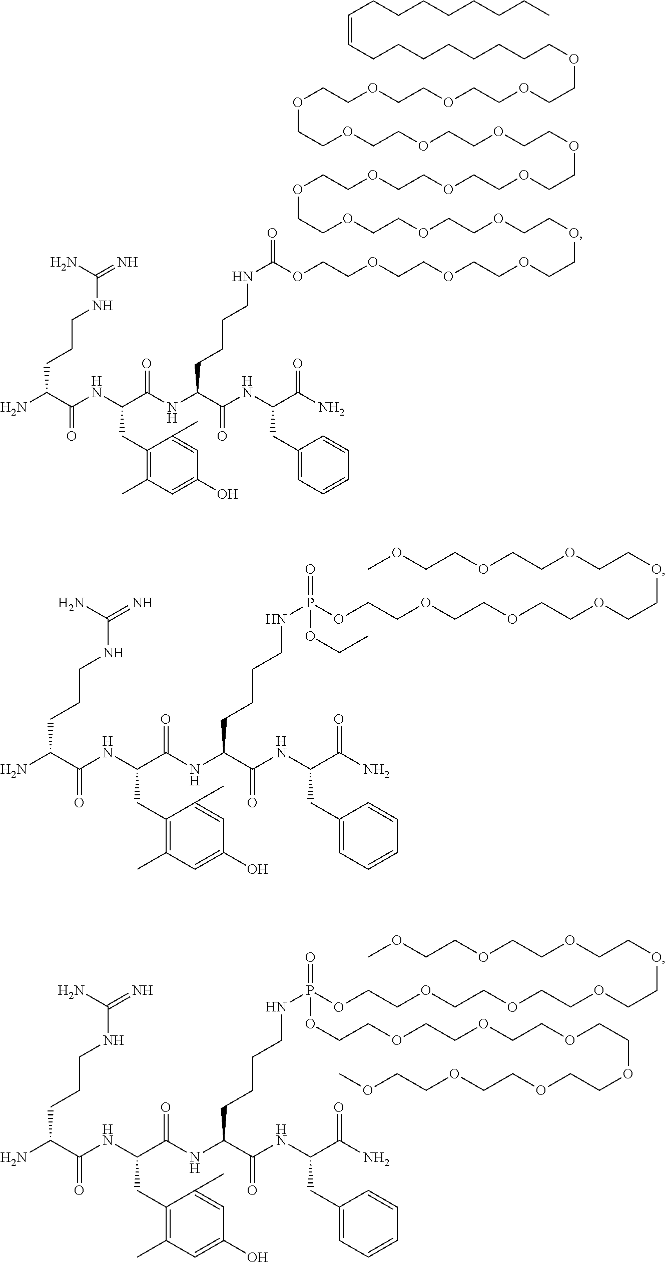 [SMILES: CCCCCCCC/C=C\CCCCCCCCOCCOCCOCCOCCOCCOCCOCCOCCOCCOCCOCCOCCOCCOCCOCCOCCOCCOCCOCCOCCOC(=O)NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O ; CCOP(=O)(NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O)OCCOCCOCCOCCOCCOCCOCCOC ; COCCOCCOCCOCCOCCOCCOCCOP(=O)(NCCCC[C@H](NC(=O)[C@H](Cc1c(C)cc(O)cc1C)NC(=O)[C@H](N)CCCNC(=N)N)C(=O)N[C@@H](Cc1ccccc1)C(N)=O)OCCOCCOCCOCCOCCOCCOCCOC]

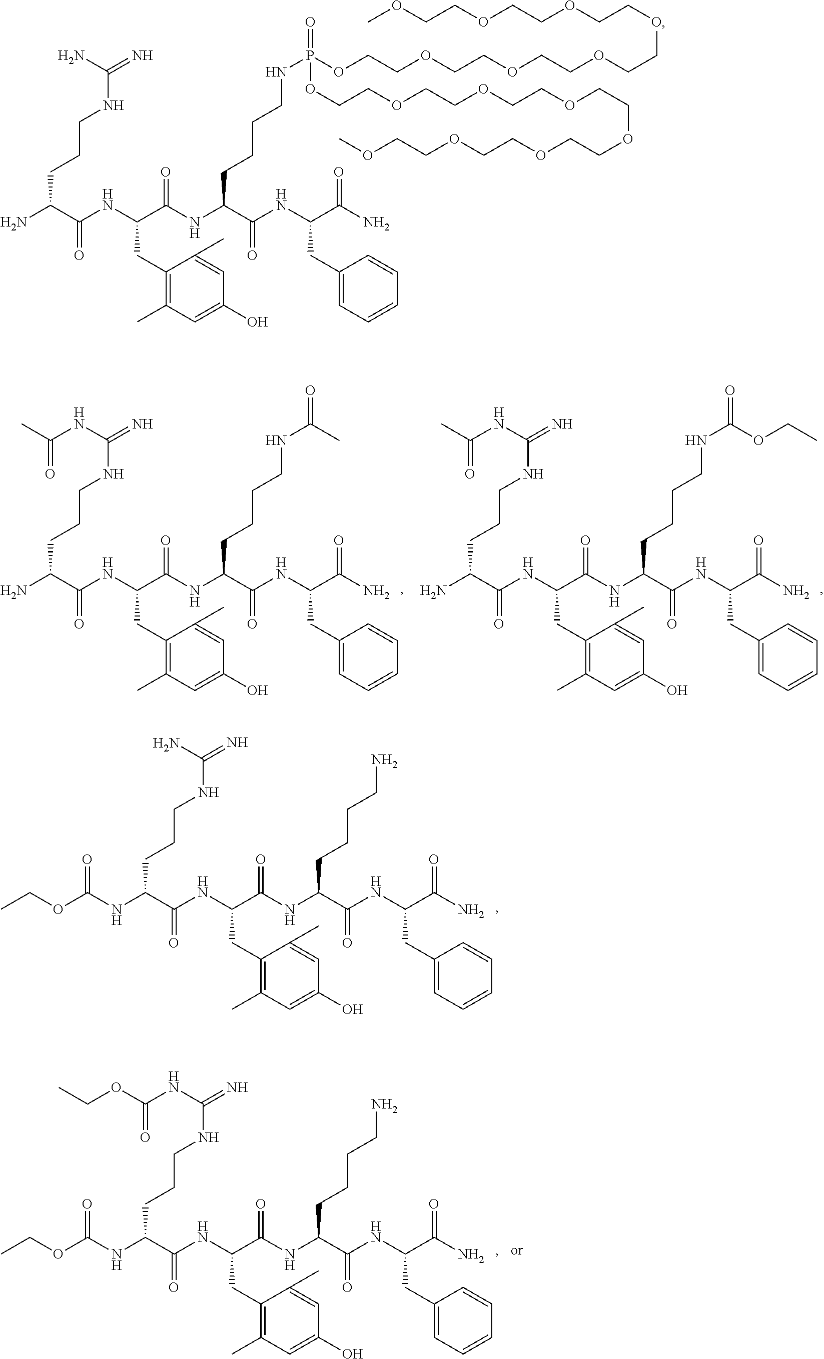

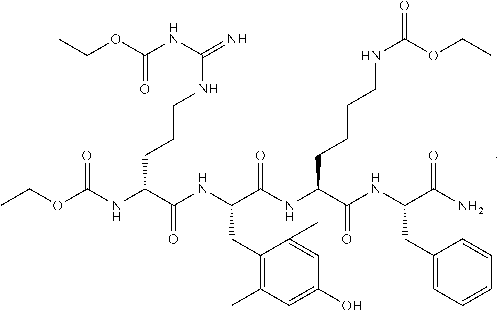 [SMILES: CCOC(=O)NCCCC[C@H](NC(=O)[C@H](Cc1c(C)cc(O)cc1C)NC(=O)[C@@H](CCCNC(=N)NC(=O)OCC)NC(=O)OCC)C(=O)N[C@@H](Cc1ccccc1)C(N)=O]

96. A compound of Formula (II): 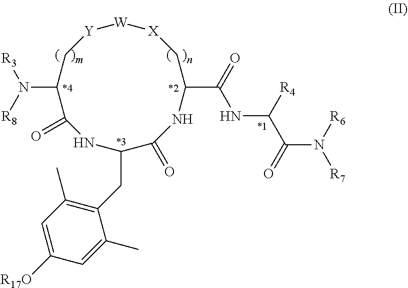
wherein:
X is —N(R₁₅)—, 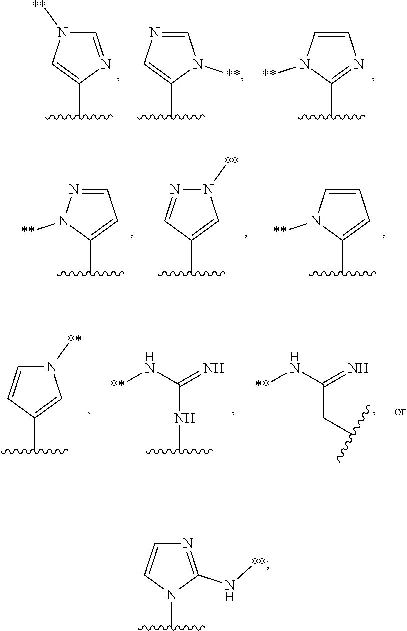
Y is —N(R₁₅)—, 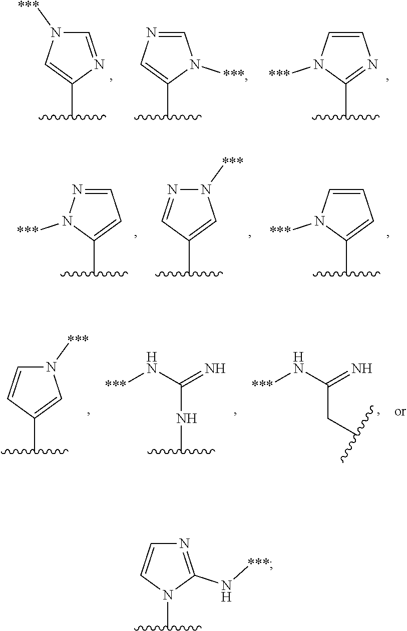
W is —C(O)—, —C(S)—, —C(R₁₆)₂—, —S(O)—, —S(O₂)—, or —P(O)[Q(R₁₀)]—;
Q is O or a bond;
R₃ and R₁₇ are independently H, alkyl, alkenyl, alkynyl, aryl, arylalkyl, arylheteroalkyl, cycloalkyl, heteroalkyl, heteroaryl, T, R₉C(O)—, R₁₀OC(O)—, R₁₁R₁₂NC(O)—, R₁₀S(O)—, R₁₀S(O)₂—, R₁₀OS(O)—, R₁₀OS(O)₂—, (R₁₁O)(R₁₂O)P(O)—, or R₁₁R₁₂N(R₉O)P(O)—;
R₄ is alkyl, cycloalkyl, aryl, arylalkyl, heteroaryl, arylheteroalkyl, T, a side-chain of a naturally or non-naturally occurring chiral amino acid, 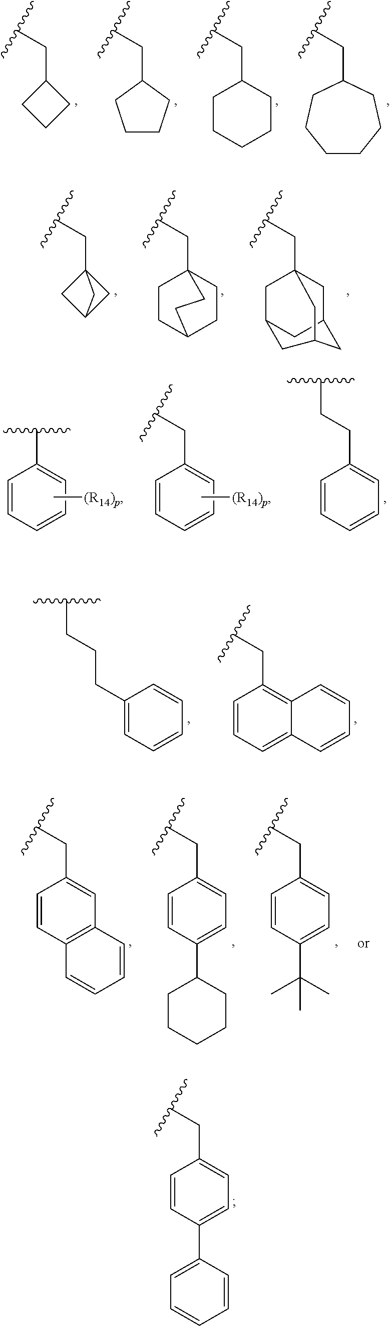
R₆ and R₇ are independently H, alkyl, or acyl; or R₆ and R₇ together with the nitrogen atom to which they are attached form a 4-6-membered heterocyclic ring;
R₈ is H, alkyl, heteroalkyl, or acyl;
R₉, R₁₁, and R₁₂ are independently H, alkyl, alkenyl, alkynyl, heteroalkyl, cycloalkyl, aryl, arylalkyl, heteroaryl, arylheteroalkyl, heteroarylheteroalkyl, or T;
R₁₁ and R₁₂ can be taken together to form a heterocyclic ring;
R₁₀ is alkyl, alkenyl, alkynyl, heteroalkyl, cycloalkyl, aryl, arylalkyl, heteroaryl, arylheteroalkyl, heteroarylheteroalkyl or T;
R₁₃ is H, methyl, ethyl, isopropyl or tert-butyl;
R₁₄ is independently D, F, Cl, Br, I, —CH₃, —OCH₃, CH₂CH₃, —OCH₂CH₃, —CCl₃, —CF₃, —C≡N, —OH, or —NO₂;
R₁₅ is H, alkyl, alkenyl, alkynyl, cycloalkyl, heteroalkyl, or acyl;
R₁₆ is alkyl, alkenyl, alkynyl, heteroalkyl, cycloalkyl, aryl, or arylalkyl;
T is —(CH₂)_{w}—(O)ₓ—[(CH₂CH₂)—O]_{q}—R₁₃;
the absolute stereochemistry at each of stereocenters *1, *2, *3 and *4 is independently R (D for an amino acid) or S (L for an amino acid);
n and m are independently 1, 2, 3, 4, 5, or 6;
p is 0, 1, 2, 3, 4, or 5;
q is an integer from 1-30 inclusive;
x is 0 or 1; and
w is 0, 1 or 2; provided that: if x is 0, then w is 0; and if w is 0, then y is 0;
“**” denotes the point of attachment of X to W; and
“***” denotes the point of attachment of W to Y.

97. The compound of claim 96, wherein X is —N(R₁₅).

98. The compound of claim 96, wherein X is

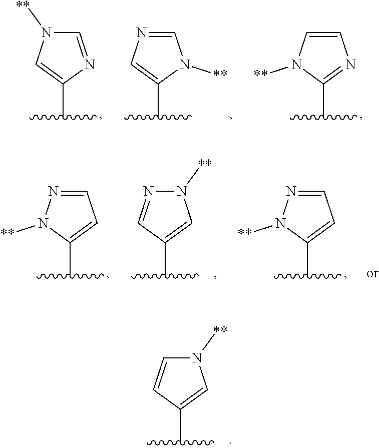

99. The compound of claim 96, wherein X is

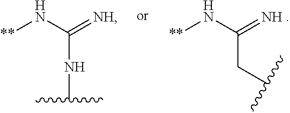

100. The compound of claim 96, wherein X is

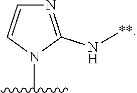

101. The compound of any one of claims 96-101, wherein Y is —N(R₁₅)—.

102. The compound of any one of claims 96-101, wherein Y is

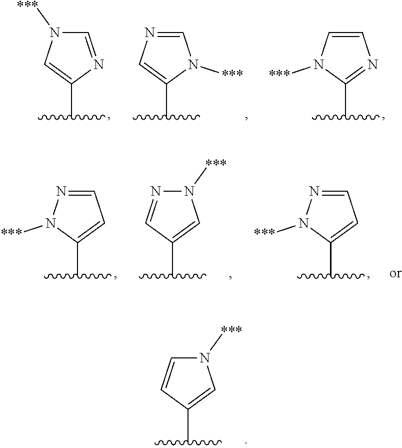

103. The compound of any one of claims 96-101, wherein Y is

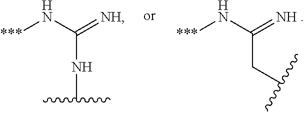

104. The compound of any one of claims 96-101, wherein Y is

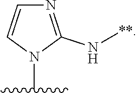

105. The compound of any one of claims 96-101, wherein W is —C(O)—.

106. The compound of any one of claims 96-101, wherein W is —C(S)—, or —C(R₁₆)₂.

107. The compound of any one of claims 96-101, wherein W is —S(O)—, or —S(O₂)—.

108. The compound of any one of claims 96-101, wherein W is —P(O)[Q(R₁₀)]—;

109. The compound of claim 108, wherein Q is O.

110. The compound of claim 108, wherein Q is a bond.

111. The compound of any one of claims 96-110, wherein R₃ is H.

112. The compound of any one of claims 96-110, wherein R₃ is alkyl, alkenyl, alkynyl, aryl, arylalkyl, arylheteroalkyl, cycloalkyl, heteroalkyl, or heteroaryl.

113. The compound of any one of claims 96-110, wherein R₃ is T.

114. The compound of claim 108, wherein R₃ is —[(CH₂CH₂)—O]_{q}—R₁₃.

115. The compound of any one of claims 96-110, wherein R₃ is R₉C(O)—, R₁₀OC(O)—, or (R₁₁O)(R₁₂O)P(O)—.

116. The compound of any one of claims 96-110, wherein R₃ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—.

117. The compound of any one of claims 96-116, wherein R₄ is alkyl, cycloalkyl, aryl, arylalkyl, heteroaryl, or arylheteroalkyl.

118. The compound of any one of claims 96-116, wherein R₄ is T.

119. The compound of claim 118, wherein R₄ is —(CH₂)—(O)—[(CH₂CH₂)—O]_{q}—R₁₃.

120. The compound of claim 118, wherein R₄ is —(CH₂)₂—(O)—[(CH₂CH₂)—O]_{q}—R₁₃.

121. The compound of any one of claims 96-116, wherein R₄ is a side-chain of a naturally or non-naturally occurring chiral amino acid.

122. The compound of any one of claims 96-116, wherein R₄ is

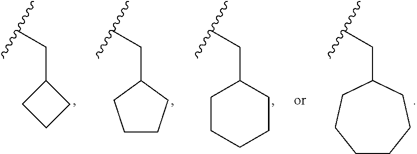

123. The compound of any one of claims 96-116, wherein R₄ is

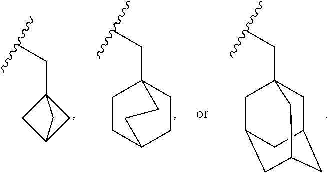

124. The compound of any one of claims 96-116, wherein R₄ is

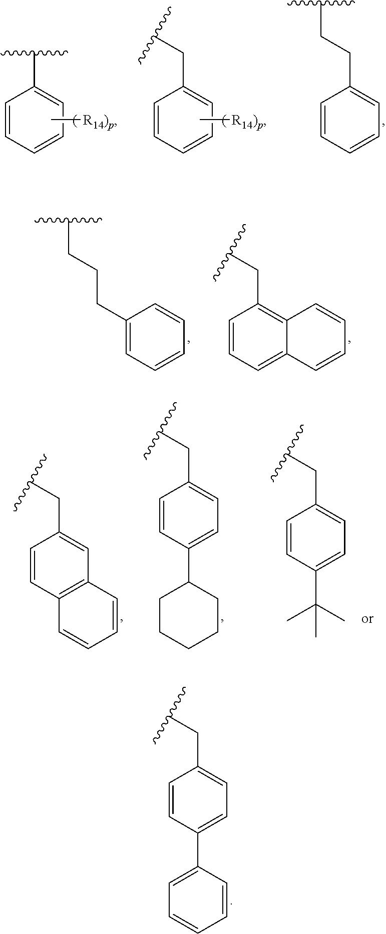

125. The compound of claim 124, wherein R₅ is 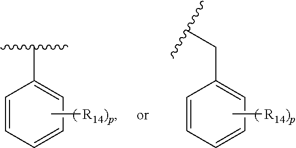
and each R₁₄ is H.

126. The compound of any one of claims 96-125, wherein R₆ is H.

127. The compound of any one of claims 96-125, wherein R₆ is alkyl or acyl.

128. The compound of any one of claims 96-127, wherein R₇ is H.

129. The compound of any one of claims 96-127, wherein R₇ is alkyl or acyl.

130. The compound of any one of claims 96-125, wherein R₆ and R₇ together with the nitrogen atom to which they are attached form a 4-6-membered heterocyclic ring.

131. The compound of any one of claims 96-130, wherein R₈ is H.

132. The compound of any one of claims 96-130, wherein R₈ is alkyl, heteroalkyl, or acyl.

133. The compound of any one of claims 96-130, wherein R₈ is H, methyl or ethyl.

134. The compound of any one of claims 96-133, wherein R₉ is H.

135. The compound of any one of claims 96-133, wherein R₉ is alkyl, alkenyl, alkynyl, heteroalkyl, cycloalkyl, aryl, arylalkyl, heteroaryl, arylheteroalkyl, or heteroarylheteroalkyl.

136. The compound of claim 135, wherein R₉ is C₁-C₈ alkyl.

137. The compound of any one of claims 96-133, wherein R₉ is T.

138. The compound of claim 137, wherein R₉ is —[(CH₂CH₂)—O]_{q}—R₁₃ and q is 1-20.

139. The compound of any one of claims 96-138, wherein R₁₀ is alkyl, alkenyl, alkynyl, heteroalkyl, cycloalkyl, aryl, arylalkyl, heteroaryl, arylheteroalkyl, or heteroarylheteroalkyl.

140. The compound of claim 139, wherein R₁₀ is C₁-C₈ alkyl.

141. The compound of any one of claims 96-138, wherein R₁₀ is T.

142. The compound of claim 140, wherein R₁₀ is —[(CH₂CH₂)—O]_{q}—R₁₃ and q is 1-20.

143. The compound of any one of claims 96-142, wherein R₁₁ is H.

144. The compound of any one of claims 96-142, wherein R₁₁ is alkyl, alkenyl, alkynyl, heteroalkyl, cycloalkyl, aryl, arylalkyl, heteroaryl, arylheteroalkyl, or heteroarylheteroalkyl.

145. The compound of claim 144, wherein R₁₁ is C₁-C₈ alkyl.

146. The compound of any one of claims 96-142, wherein R₁₁ is T.

147. The compound of claim 146, wherein R₁₁ is —[(CH₂CH₂)—O]_{q}—R₁₃ and q is 1-20.

148. The compound of any one of claims 96-147, wherein R₁₂ is H.

149. The compound of any one of claims 96-147, wherein R₁₂ is alkyl, alkenyl, alkynyl, heteroalkyl, cycloalkyl, aryl, arylalkyl, heteroaryl, arylheteroalkyl, or heteroarylheteroalkyl.

150. The compound of claim 149, wherein R₁₂ is C₁-C₈ alkyl.

151. The compound of any one of claims 96-147, wherein R₁₂ is T.

152. The compound of claim 151, wherein R₁₂ is —[(CH₂CH₂)—O]_{q}—R₁₃ and q is 1-20.

153. The compound of any one of claims 96-152, wherein R₁₃ is H.

154. The compound of any one of claims 96-152, wherein R₁₃ is methyl, ethyl, isopropyl, or tert-butyl.

155. The compound of any one of claims 96-124 and 126-154, wherein R₁₄ is D.

156. The compound of any one of claims 96-124 and 126-154, wherein R₁₄ is F, Cl, Br, I, —CCl₃, or —CF₃.

157. The compound of any one of claims 96-124 and 126-154, wherein R₁₄ is —CH₃, —OCH₃, CH₂CH₃, —OCH₂CH₃, —C≡N, —OH, or —NO₂.

158. The compound of any one of claims 96-157, wherein R₁₅ is H.

159. The compound of any one of claims 96-157, wherein R₁₅ is alkyl, alkenyl, alkynyl, cycloalkyl, heteroalkyl, or acyl.

160. The compound of claim 159, wherein R₁₅ is methyl, ethyl, isopropyl or tert-butyl.

161. The compound of any one of claims 96-160, wherein R₁₆ is alkyl, alkenyl, alkynyl, or heteroalkyl.

162. The compound of claim 160, wherein R₁₆ is methyl, ethyl, isopropyl or tert-butyl.

163. The compound of any one of claims 96-160, wherein R₁₆ is cycloalkyl, aryl, or arylalkyl.

164. The compound of any one of claims 96-163, wherein R₁₇ is H.

165. The compound of any one of claims 96-163, wherein R₁₇ is alkyl, alkenyl, alkynyl, aryl, arylalkyl, arylheteroalkyl, cycloalkyl, heteroalkyl, or heteroaryl.

166. The compound of any one of claims 96-163, wherein R₁₇ is —(CH₂)—(O)—[(CH₂CH₂)—O]_{q}—R₁₃ or —(CH₂)₂—(O)—[(CH₂CH₂)—O]_{q}—R₁₃.

167. The compound of any one of claims 96-163, wherein R₁₇ is R₉C(O)—, R₁₀OC(O)—, or (R₁₁O)(R₁₂O)P(O)—.

168. The compound of any one of claims 96-163, wherein R₁₇ is R₁₁R₁₂NC(O)—, R₁₀S(O)—, R₁₀S(O)₂—, R₁₀OS(O)—, R₁₀OS(O)₂—, or R₁₁R₁₂N(R₉O)P(O)—.

169. The compound of any one of claims 96-163, wherein R₁₇ is methyl or ethyl

170. The compound of any one of claims 96-169, wherein n is 1, 2, 3 or 4.

171. The compound of any one of claims 96-169, wherein n is 5, or 6.

172. The compound of any one of claims 96-169, wherein m is 1, 2, 3 or 4.

173. The compound of any one of claims 96-169, wherein m is 5, or 6.

174. The compound of any one of claims 96-173, wherein the stereochemistry at the carbon atom labeled *4 is D, the stereochemistry at the carbon atom labeled *3 is L, the stereochemistry at the carbon atom labeled *2 is L, and the stereochemistry at the carbon atom labeled *1 is L.

175. The compound of any one of claims 96-173, wherein the stereochemistry at the carbon atom labeled *4 is L, the stereochemistry at the carbon atom labeled *3 is D, the stereochemistry at the carbon atom labeled *2 is D, and the stereochemistry at the carbon atom labeled *1 is D.

176. The compound of any one of claims 96-173, wherein the stereochemistry at the carbon atom labeled *4 is D, the stereochemistry at the carbon atom labeled *3 is D, the stereochemistry at the carbon atom labeled *2 is D, and the stereochemistry at the carbon atom labeled *1 is D.

177. The compound of any one of claims 96-173, wherein the stereochemistry at the carbon atom labeled *4 is L, the stereochemistry at the carbon atom labeled *3 is L, the stereochemistry at the carbon atom labeled *2 is L, and the stereochemistry at the carbon atom labeled *1 is L.

178. The compound of any one of claims 96-173, wherein the stereochemistry at the carbon atom labeled *4 is D, the stereochemistry at the carbon atom labeled *3 is L, the stereochemistry at the carbon atom labeled *2 is D, and the stereochemistry at the carbon atom labeled *1 is L.

179. The compound of any one of claims 96-173, wherein the stereochemistry at the carbon atom labeled *4 is L, the stereochemistry at the carbon atom labeled *3 is D, the stereochemistry at the carbon atom labeled *2 is L, and the stereochemistry at the carbon atom labeled *1 is D.

180. The compound of claim 96, wherein the compound is

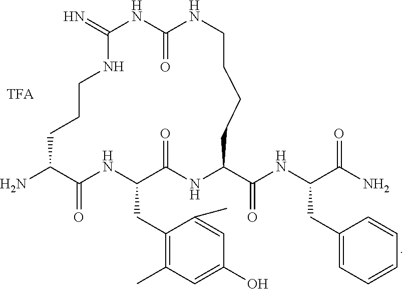 [SMILES: Cc1cc(O)cc(C)c1C[C@@H]1NC(=O)[C@H](N)CCCNC(=N)NC(=O)NCCCC[C@@H](C(=O)N[C@@H](Cc2ccccc2)C(N)=O)NC1=O]

181. The compound of claim 96, wherein the compound is 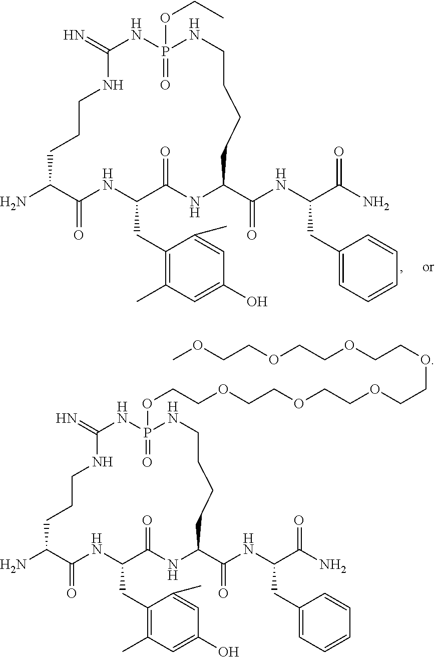 [SMILES: CCOP1(=O)NCCCC[C@@H](C(=O)N[C@@H](Cc2ccccc2)C(N)=O)NC(=O)[C@H](Cc2c(C)cc(O)cc2C)NC(=O)[C@H](N)CCCNC(=N)N1 ; COCCOCCOCCOCCOCCOCCOCCOP1(=O)NCCCC[C@@H](C(=O)N[C@@H](Cc2ccccc2)C(N)=O)NC(=O)[C@H](Cc2c(C)cc(O)cc2C)NC(=O)[C@H](N)CCCNC(=N)N1]
